(19) United States
(12) Patent Application Publication
(10) Pub. No.: US 2014/0371285 A1
(43) Pub. Date: Dec. 18, 2014

(54) THERAPEUTIC COMPOUNDS AND RELATED METHODS OF USE

(71) Applicants: Kevin Sprott, Cambridge, MA (US); Michael Lewis, Andover, MA (US); Hyeongwook Choi, Andover, MA (US); Frank Fang, Andover, MA (US); Mingde Shan, Andover, MA (US); Tsvetelina I. Lazarova, Brookline, MA (US); Lin Li, Shanghai (CN); M. Arshad Siddiqui, Montreal (CA); Robin Larouche-Gauthier, Montreal (CA); Alexandre Lemire, Montreal (CA)

(72) Inventors: Kevin Sprott, Cambridge, MA (US); Michael Lewis, Andover, MA (US); Hyeongwook Choi, Andover, MA (US); Frank Fang, Andover, MA (US); Mingde Shan, Andover, MA (US); Tsvetelina I. Lazarova, Brookline, MA (US); Lin Li, Shanghai (CN); M. Arshad Siddiqui, Montreal (CA); Robin Larouche-Gauthier, Montreal (CA); Alexandre Lemire, Montreal (CA)

(21) Appl. No.: 14/370,415

(22) PCT Filed: Jan. 7, 2013
(86) PCT No.: PCT/US2013/020585

§ 371 Date: Jul. 2, 2014

Related U.S. Application Data
(60) Provisional application No. 61/613,127, filed on Mar. 20, 2012.

(30) Foreign Application Priority Data
Jan. 6, 2012 (CN) 201210005407.4

(51) Int. Cl. C07D 493/20

(52) U.S. Cl. CPC C07D 493/20
USPC 514/406; 549/343; 514/460; 549/344; 514/456; 548/365.7

(57) ABSTRACT

Salinomycin analogs and pharmaceutically acceptable compositions containing salinomycin analogs. Dosage forms and kits comprising salinomycin analogs and pharmaceutically acceptable compositions containing salinomycin analogs. Methods of using salinomycin analogs, pharmaceutically acceptable compositions, dosage forms, and kits for the treatment of proliferative diseases, e.g., cancer, or microbial infections in a subject

### CROSS-REFERENCE TO RELATED APPLICATIONS

This application claims priority to Chinese Patent Application No. CN201210005407.4, filed Jan. 6, 2012 and U.S. Provisional application Ser. No. 61/613,127, filed Apr. 6, 2012, each of which is incorporated by reference herein in its entirety.

### BACKGROUND OF INVENTION

Research suggests tumor formation and growth are influenced by a minor subpopulation of cancer cells including cancer stem cells (CSCs) and mesenchymal cells, e.g., mesenchymal cancerous cells. Cancer stem cells (CSCs) are cells within a tumor mass that have the capacity to seed and generate secondary tumors, and are implicated in cancerous processes such as metastasis and relapse. Mesenchymal cells are undifferentiated loose cells that can easily migrate throughout a system of a subject, and given the proper environment, proliferate rapidly. Recent research suggests that while conventional cancer therapies (e.g., surgery, radiation, chemotherapy, hormone therapies) can eradicate the bulk of tumors, CSCs and/or mesenchymal cells are often left behind. The lingering CSCs and/or mesenchymal cells can become the nuclei for new tumors, either in the original tissue or elsewhere in the subject.

Thus, there is a need for drugs that specifically and selectively target CSCs and/or mesenchymal cells. Such drugs could be used alone, or in combination with traditional cancer therapies (e.g., surgery, radiation, chemotherapy, hormone therapies) to destroy tumors and avoid relapse or metastasis.

Treatments using these drugs, e.g., that target CSCs and/or mesenchymal cells, will be useful in treating cancer and avoiding metastasis and relapse. Such therapies will also benefit from improved methods of detecting stem cells, thereby allowing subjects with greater risk for relapse or metastasis to be identified. Methods of detecting stem cells will additionally provide the capacity to personalize therapies for subjects having been identified with cancer, or at risk for developing cancer.

### SUMMARY OF INVENTION

Described herein are compounds that kill, bind, inhibit the growth of, or prevent the proliferation of, cancer stem cells and/or mesenchymal cells, and pharmaceutically acceptable salts and hydrates thereof. Also described are compositions, pharmaceutical preparations, e.g., dosage forms, and kits comprising the compounds described herein. Methods of treatment using these compounds, e.g., for the treatment of a subject identified as having cancer or being infected with a microorganism, are described. The treatments may be in combination with screening methods in which the subject has been identified as having a disorder associated with cancer stem cells and/or mesenchymal cells. In some cases, the compounds, compositions, pharmaceutical preparations, dosage forms, etc. are administered in combination with other cancer therapies (e.g., surgery, radiation, chemotherapy, hormone therapies).

In one aspect, the invention features a compound of Formula I:

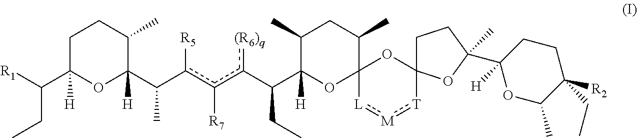

wherein R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano; R₂ is —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; L-M-T together form a structure selected from —C(R₃)ₙ—C(R₃)ₙ—C(R₃)ₙ—, —CR₃═CR₃—C(R₃)ₙ—, and —C(R₃)ₙ—CR₃═CR₃—; or L-M, M-T, or L-M-T, and one to three additional —C(R₃)ₙ—, —O—, —NR₁₁—, or —S— to which they are bound, together form a 3-6 membered cyclyl, heterocyclyl, aryl, or heteroaryl ring, wherein n is independently 1 or 2; each R₃ is independently H, halo, oxo, —OR₁₀, —SR₁₀, —OC(O)R₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl; R₅ is H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; R₆ is independently H, oxo, —OR_{R}), —SR₁₀, —COR₁₀, —CNR₁₁R₁₂, —C(O)R₁₀, —C(O)OR₁₀, —C(O)NR₁₁R₁₂, —OC(O)R₁₀, —OS(O)R₁₀, —OC(O)OR₁₀, —OS(O)OR₁₀, —NR₁₁R₁₂, ═NR₁₁, —OC(O)NR₁₁R₁₂, —SC(O)NR₁₁R₁₂, halo (e.g., F, Cl, Br, I), —NH₂, cyano, C₁-C₈ alkyl, cyclylalkyl, or aryl; R₅ and R₆ together may optionally form a substituted or unsubstituted 5-8 membered cyclyl, heterocyclyl, aryl, or heteroaryl ring; R₇ is H, halo, C₁-C₈ alkyl, or C₁-C₈ heteroalkyl; R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain; R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR₁₃, —C(O)OR₁₃, —OC(O)R₁₃, —C(O)R₁₃, —S(O)R₁₃, —S(O₂)R₁₃, —NR₁₃R₁₄, cyano, or an amino acid side chain; R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano; R₁₅ and R₁₆ are each independently ═O, ═S, —OR₁₇, —SR₁₇, or —NR₁₇R₁₈, provided R₁₅ and R₁₆ are not both double-bonded moieties; R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl; R₁₉ is —O—, —S—, —NR₁₇—, —N(OH)—, or —N(OR₁₀)—; and q is 1 or 2; provided that when R₁ is —C(O)OH, n is 2, q is 1, R₆ is oxo, and R₇ is methyl, R₂, R₃, and R₅ are not all hydroxy; provided that when R₁ is —C(O)OH, R₆ is oxo, R₇ is methyl, and R₃ and R₅ are hydroxy, R₂ is not benzoxy or benzyloxy; and provided that when R₁ is —C(O)OH, R₆ is oxo, R₇ is methyl, and R₂ is hydroxyl, R₃ or R₅ are not —OCH₂Cl, —OCH₂Br, or —OC(O)CH₂Cl.

In one embodiment, the invention features a compound of Formula II:

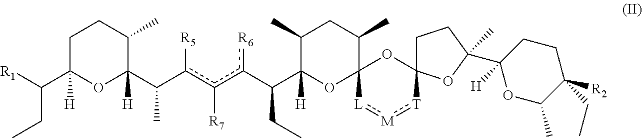

wherein R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano; R₂ is —OR₁₀, —SR₁₀, —OC(O)R₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; L-M-T together form a structure selected from —C(R₃)₂—C(R₃)₂—C(R₃)₂—, —CR₃═CR₃—C(R₃)₂—, and —C(R₃)₂—CR₃═CR₃—; or L-M, M-T, or L-M-T, and one to three additional —C(R₃)₂—, —O—, —NR₁₁—, or —S— to which they are bound, together form a 3-6 membered cyclyl, heterocyclyl, aryl, or heteroaryl ring; each R₃ is independently H, halo, oxo, —OR₁₀, —SR₁₀, —OC(O)R₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl; R₅ is H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; R₆ is H, oxo, —OR₁₀, —SR₁₀, —OC(O)R₁₀, —OS(O)R₁₀, —OC(O)OR₁₀, —OS(O)OR₁₀, —NR₁₁R₁₂, ═NR₁₁, —OC(O)NR₁₁R₁₂, —SC(O)NR₁₁R₁₂, halo (e.g., F, Cl, Br, I), —NH₂, cyano, cyclylalkyl, or aryl; R₅ and R₆ together may optionally form a substituted or unsubstituted 5-8 membered cyclyl, heterocyclyl, aryl, or heteroaryl ring; R₇ is H, halo, C₁-C₈ alkyl, or C₁-C₈ heteroalkyl; R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain; R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR₁₃, —C(O)OR₁₃, —OC(O)R₁₃, —C(O)R₁₃, —S(O)R₁₃, —S(O₂)R₁₃, —NR₁₃R₁₄, cyano, or an amino acid side chain; R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano; R₁₅ and R₁₆ are each independently ═O, ═S, —OR₁₇, —SR₁₇, or —NR₁₇R₁₈, provided R₁₅ and R₁₆ are not both double-bonded moieties; R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl; and R₁₉ is —O—, —S—, —NR₁₇—, —N(OH)—, or —N(OR₁₀)—; provided that when R₁ is —C(O)OH, R₆ is oxo, and R₇ is methyl, R₂, R₃, and R₅ are not all hydroxy; provided that when R₁ is —C(O)OH, R₆ is oxo, R₇ is methyl, and R₃ and R₅ are hydroxy, R₂ is not benzoxy or benzyloxy; and provided that when R₁ is —C(O)OH, R₆ is oxo, R₇ is methyl, and R₂ is hydroxyl, R₃ or R₅ are not —OCH₂Cl, —OCH₂Br, or —OC(O)CH₂Cl.

In one embodiment, the invention features a compound of Formula II:

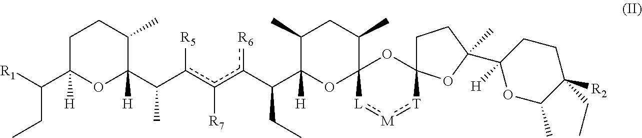

wherein R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano; R₂ is —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; L-M-T together form a structure selected from —C(R₃)₂—C(R₃)₂—C(R₃)₂— and —C(R₃)₂—CR₃═CR₃—; or L-M, M-T, or L-M-T, and one to three additional —C(R₃)₂—, —O—, —NR₁₁—, or —S— to which they are bound, together form a 3-6 membered cyclyl, heterocyclyl, aryl, or heteroaryl ring; each R₃ is independently H, halo, oxo, —OR₁₀, —SR₁₀, —OC(O)R₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl; R₅ is H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; R₆ is H, oxo, —OR₁₀, —SR₁₀, —COR₁₀, —CNR₁₁R₁₂, —C(O)R₁₀, —C(O)OR₁₀, —C(O)NR₁₁R₁₂, —OC(O)R₁₀, —OS(O)R₁₀, —OC(O)OR₁₀, —OS(O)OR₁₀, —NR₁₁R₁₂, ═NR₁₁, —OC(O)NR₁₁R₁₂, —SC(O)NR₁₁R₁₂, halo (e.g., F, Cl, Br, I), —NH₂, cyano, C₁-C₈ alkyl, cyclylalkyl, or aryl; R₅ and R₆ together may optionally form a substituted or unsubstituted 5-8 membered cyclyl, heterocyclyl, aryl, or heteroaryl ring; R₇ is H, halo, C₁-C₈ alkyl, or C₁-C₈ heteroalkyl; R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain; R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR₁₃, —C(O)OR₁₃, —OC(O)R₁₃, —C(O)R₁₃, —S(O)R₁₃, —S(O₂)R₁₃, —NR₁₃R₁₄, cyano, or an amino acid side chain; R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano; R₁₅ and R₁₆ are each independently ═O, ═S, —OR₁₇, —SR_{N}, or —NR₁₇R₁₈ provided R₁₅ and R₁₆ are not both double-bonded moieties; R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl; and R₁₉ is —O—, —S—, —NR₁₇—, —N(OH)—, or —N(OR₁₀)—.

In one embodiment, the invention features a compound of Formula II:

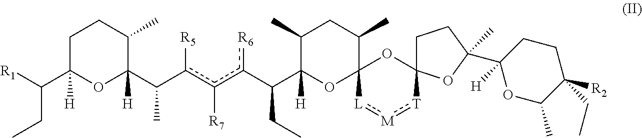

wherein, R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano; R₂ is —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; L-M-T together form a structure selected from —C(R₃)₂—C(R₃)₂—C(R₃)₂—, —CR₃═CR₃—C(R₃)₂—, and —C(R₃)₂—CR₃═CR₃—; or L-M, M-T, or L-M-T, and one to three additional —C(R₃)₂—, —O—, —NR₁₁—, or —S— to which they are bound, together form a 3-6 membered cyclyl, heterocyclyl, aryl, or heteroaryl ring; each R₃ is independently H, halo, oxo, —SR₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl; R₅ is H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; R₆ is independently H, oxo, —OR₁₀, —SR₁₀, —COR₁₀, —CNR₁₁R₁₂, —C(O)R₁₀, —C(O)OR₁₀, —C(O)NR₁₁R₁₂, —OC(O)R₁₀, —OS(O)R₁₀, —OC(O)OR₁₀, —OS(O)OR₁₀, —NR₁₁R₁₂, ═NR₁₁, —OC(O)NR₁₁R₁₂, —SC(O)NR₁₁R₁₂, halo (e.g., F, Cl, Br, I), —NH₂, cyano, C₁-C₈ alkyl, cyclylalkyl, or aryl; R₅ and R₆ together may optionally form a substituted or unsubstituted 5-8 membered cyclyl, heterocyclyl, aryl, or heteroaryl ring; R₇ is H, halo, C₁-C₈ alkyl, or C₁-C₈ heteroalkyl; R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain; R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR₁₃, —C(O)OR₁₃, —OC(O)R₁₃, —C(O)R₁₃, —S(O)R₁₃, —S(O₂)R₁₃, —NR₁₃R₁₄, cyano, or an amino acid side chain; R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano; R₁₅ and R₁₆ are each independently ═O, ═S, —OR₁₇, —SR₁₇, or —NR₁₇R₁₈ provided R₁₅ and R₁₆ are not both double-bonded moieties; R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl; and R₁₉ is —O—, —S—, NR₁₇—, N(OH)—, or N(OR₁₀)—.

In one aspect, the invention features a composition, e.g., a pharmaceutical composition, comprising a compound of formula (I).

In one aspect, the invention features a dosage form, e.g., a pharmaceutical dosage form, comprising a compound of formula (I). In some embodiments the dosage form may be administered to a subject intravenously or as a subcutaneous bolus.

In one aspect, the invention features a kit comprising a compound of formula (I), as well as kits comprising pharmaceutical compositions or dosage forms containing a compound of formula (I), e.g., pharmaceutical compositions or dosage forms described herein. In some embodiments, the kit additionally comprises a pharmaceutically acceptable diluent or instructions for administration of the compound, pharmaceutical composition or dosage form.

In one aspect, the invention features method of regulating cell proliferation in a subject in need thereof. The method comprises administering an effective amount of a compound a compound of formula (I). In some embodiments, the method comprises administering to the subject a pharmaceutical composition or dosage form containing an effective amount of a compound of formula (I), e.g., a pharmaceutical composition or dosage form described herein.

In one embodiment, the invention features a method of treating cancer in a subject comprising administering a compound of formula (I). In some embodiments, the method comprises administering to the subject a pharmaceutical composition or dosage form containing an effective amount of a compound of formula (I), e.g., a pharmaceutical composition or dosage form described herein. In some embodiments, the method further comprises administering an additional cancer therapy (e.g., surgery, radiation, chemotherapy, hormonal therapy, vaccines, antibodies, gene therapy or other targeted therapies).

In one aspect, the invention features a method of inhibiting the proliferation of cancer stem cells or mesenchymal cells, comprising contacting the cancer stem cells or mesenchymal cells with a compound of formula (I).

In one aspect, the invention features method of regulating or reducing the growth of microorganisms in a subject, comprising administering a compound of formula (I).

In one aspect, the invention features a method of identifying or selecting a subject that would benefit from the administration of a compound of formula (I), or pharmaceutical compositions or dosage forms thereof, comprising screening the subject for one or more biomarkers selected from the biomarkers described herein.

In some embodiments, a compound as described herein is active in the ALDEFLUOR assay (e.g., as described herein).

In some embodiments, a subject identified with one or more biomarkers selected from the biomarkers described herein will be administered a compound of formula (I), or a pharmaceutical composition or dosage form thereof.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting.

### DEFINITIONS

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents).

The term “alkenyl” refers to a straight or branched hydrocarbon chain containing 2-12 carbon atoms (unless otherwise noted) and having one or more double bonds. Examples of alkenyl groups include, but are not limited to, alyl, propenyl, 2-butenyl, 3-hexenyl and 3-octenyl groups. One of the double bond carbons may optionally be the point of attachment of the alkenyl substituent.

The term “alkenylene” refers to a divalent alkenyl, e.g. —CH═CH—, —CH₂—CH═CH—, and CH═CH—CH₂—.

The term “alkynyl” refers to a straight or branched hydrocarbon chain containing 2-12 carbon atoms (unless otherwise noted) and characterized in having one or more triple bonds. Examples of alkynyl groups include, but are not limited to, ethynyl, propargyl, and 3-hexynyl. One of the triple bond carbons may optionally be the point of attachment of the alkynyl substituent.

The term “alkynylene” refers to a divalent alkynyl, e.g. —CH≡CH—, —CH₂—CH≡CH—, and CH═CH—CH₂—.

The terms “alkoxyl” or “alkoxy” as used herein refers to an alkyl group, as defined below, having an oxygen radical attached thereto. Representative alkoxy groups include methoxy, ethoxy, propyloxy, tert-butoxy and the like. The term “alkoxyalkyl” refers to an alkyl in which one or more hydrogen atoms are replaced by an alkoxy group.

An “ether” is two hydrocarbons covalently linked by an oxygen.

The term “alkyl” refers to the radical of saturated aliphatic groups, including straight-chain (linear) alkyl groups, and branched-chain alkyl groups. In preferred embodiments, a straight chain or branched chain alkyl has 12 or fewer carbon atoms in its backbone (unless otherwise noted) e.g., from 1-12, 1-8, 1-6, or 1-4. Exemplary alkyl moieties include methyl, ethyl, propyl (e.g., n-propyl or isopropyl), butyl (e.g., n-butyl, isobutyl or t-butyl), pentyl (e.g., n-pentyl, isopentyl or pentan-3-yl), hexyl and hepty.

The term “alkylene” refers to a divalent alkyl, e.g., —CH₂—, —CH₂CH₂—, and —CH₂CH₂CH₂—.

The term “alkoxylene” refers to an alkylene wherein a CH₂ is substituted with an oxygen. For example, an aryl alkoxylene refers to a group with an alkylene attached to an aryl group through an oxygen, an optionally substituted heteroaryl alkoxylene refers to a group with an alkylene attached to an heteroaryl group through an oxygen.

The term “amino” refers to —NH₂.

The term “alkylamino” refers to —NH(alkyl) and —N(alkyl)₂ radicals respectively.

The term “aralkylamino” refers to a —NH(aralkyl) radical. The term alkylaminoalkyl refers to a (alkyl)NH-alkyl-radical; the term dialkylaminoalkyl refers to a (alkyl)₂N-alkyl-radical.

The term “amido” refers to a —NHC(O)— or —C(O)NH₂ substituent.

The term “aryl” refers to a 6-carbon monocyclic, 10-carbon bicyclic, or 14-carbon tricyclic aromatic ring system wherein 0, 1, 2, 3, or 4 atoms of each ring may be substituted by a substituent. Examples of aryl moieties include, but are not limited to, phenyl, naphthyl and the like.

The term “arylalkyl” refers to alkyl substituted with an aryl. Exemplary aralkyls include but are not limited to benzyl, 1-phenylethyl, 2-phenylethyl, 3-phenylpropyl, 9-fluorenyl, benzhydryl, phenethyl, and trityl groups. The term “arylalkenyl” refers to an alkenyl substituted with an aryl. The term “arylalkynyl” refers to an alkynyl substituted with an aryl. Terms such as “arylC₂-C₆alkyl” are to be read as a further limitation on the size of the alkyl group. The term “arylalkoxy” refers to an alkoxy substituted with aryl. The term “arylenyl” refers to a divalent aryl (i.e., —Ar—).

The terms “cycloalkyl” or “cyclyl” as employed herein include saturated and partially unsaturated cyclic hydrocarbon groups having 3 to 12 carbons, preferably 3 to 8 carbons, and more preferably 3 to 6 carbons, wherein the cycloalkyl group may be optionally substituted. Exemplary cyclyl groups include, without limitation, cyclopropyl, cyclobutyl, cyclopentyl, cyclopentenyl, cyclohexyl, cyclohexenyl, cycloheptyl, and cyclooctyl. Cyclyl moieties also include both bridged and fused ring systems. Cyclyl groups also include those that are fused to additional ring systems, which may be saturated or unsaturated. A cyclyl group may thus be a bicyclic group in which one ring is saturated or partially unsaturated and the other is fully unsaturated (e.g., indanyl).

The term “cyclylalkyl” as used herein, refers to an alkyl group substituted with a cyclyl group. Cyclylalkyl includes groups in which more than one hydrogen atom of an alkyl group has been replaced by a cyclyl group.

The term “cycloalkylalkyl” as used herein, refers to an alkyl group substituted with a cycloalkyl group.

The term “halo” or “halogen” refers to any radical of fluorine, chlorine, bromine or iodine.

The term “haloalkyl” refers to an alkyl group that may have any number of hydrogens available on the group replaced with a halogen atom. Representative haloalkyl groups include but are not limited to: —CH₂Cl, —CH₂ClCF₃, —CHBr₂, —CF₃, —CH₂F, —CHF₂, and —CH₂CF₃. The term “fluoroalkyl” refers to an alkyl group that may have any number of hydrogens available on the group replaced with a fluorine atom. Representative fluoroalkyl groups include but are not limited to: —CH₂F, —CH₂FCF₃, —CHF₂ or —CF₃. The term “haloalkoxy” refers to an alkoxy group that may have any number of hydrogen atoms available on the alkyl group replaced with a halogen atom. Representative haloalkoxy groups include but are not limited to: —OCH₂Cl, —OCH₂ClCF₃, —OCHBr₂, —OCHF₂ or OCF₃. The term “fluoroalkoxy” refers to an alkoxy group that may have any number of hydrogens available on the group replaced with a fluorine atom. Representative fluoroalkoxy groups include but are not limited to: —OCH₂F, —OCH₂FCF₃, —OCHF₂ or —OCF₃.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, oxazolyl and the like. The term “heteroarylalkyl” or the term “heteroaralkyl” refers to an alkyl substituted with a heteroaryl. The term “heteroarylalkenyl” refers to an alkenyl substituted with a heteroaryl. The term “heteroarylalkynyl” refers to an alkynyl substituted with a heteroaryl. The term “heteroarylalkoxy” refers to an alkoxy substituted with heteroaryl. A heteroaryl group may be mono-, bi-, tri-, or polycyclic, preferably mono-, bi-, or tricyclic, more preferably mono- or bicyclic. When a heteroaryl is substituted by a hydroxy group, it also includes its corresponding tautomer. The term “heteroaryl,” as used herein, also includes groups in which a heteroaromatic ring is fused to one or more aryl rings. Nonlimiting examples of heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. The term “heteroaryl” may be used interchangeably with the terms “heteroaryl ring”, “heteroaryl group,” or “heteroaromatic,” any of which terms include rings that are optionally substituted. A ring nitrogen atom of a heteroaryl may be oxidized to form the corresponding N-oxide compound. A nonlimiting example of such a heteroaryl having an oxidized ring nitrogen atom is N-oxopyridyl.

The term “heteroarylalkyl” refers to an alkyl group substituted by a heteroaryl. Heteroarylalkyl includes groups in which more than one hydrogen atom has been replaced by a heteroaryl group.

As used herein, the terms “heterocycle,” “heterocyclyl” and “heterocyclic ring” are used interchangeably and refer to a stable 3- to 8-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2/y-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydropyranyl, tetrahydrothienyl, piperidinyl, decahydroquinolinyl, oxazolidinyl, piperazinyl, dioxanyl, dioxolanyl, diazepinyl, oxazepinyl, thiazepinyl, morpholinyl, and thiomorpholinyl. A heterocyclyl group may be mono-, bi-, tri-, or polycyclic, preferably mono-, bi-, or tricyclic, more preferably mono- or bicyclic. Additionally, a heterocyclic ring also includes groups in which the heterocyclyl ring is fused to one or more aryl, heteroaryl or cyclyl rings. A ring nitrogen atom of a heterocyclic ring also may be oxidized to form the corresponding N-hydroxy compound.

The term “heterocyclylalkyl” refers to an alkyl group substituted by a heterocyclyl. Heterocyclylalkyl includes groups in which more than one hydrogen atom has been replaced by a heterocyclyl group.

The terms “hetaralkyl” and “heteroaralkyl”, as used herein, refers to an alkyl group substituted with a heteroaryl group. Exemplary heteroaralkyl groups include but are not limited to methylpyridyl or methylpyrimidyl.

The term “heterocyclyl” or “heterocyclylalkyl” refers to a nonaromatic 5-8 membered monocyclic, 5-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and include both bridged and fused ring systems. The term “heterocyclylalkyl” refers to an alkyl substituted with a heterocyclyl.

The term “heterocyclylalkyl”, as used herein, refers to an alkyl group substituted with a heterocycle group.

The term “heteroalkyl,” as used herein, refers to a saturated or unsaturated, straight (linear) or branched chain aliphatic group, wherein one or more of the carbon atoms in the chain are independently replaced by a heteroatom. Exemplary heteroatoms include O, S, N, and P.

Aralkyl, heteroalkyl, etc groups described as optionally substituted, it is intended that either or both aryl, alkyl or heteroraryl and alkyl may be independent optionally substituted or unsubstituted.

The term “hydroxyalkyl” refers to an alkyl in which one or more hydrogen atoms are replaced by a hydroxy group.

The term “imino” refers to a substituted or unsubstituted nitrogen (e.g., NH) with a double bond to a carbon (—C═N—), wherein the carbon may be part of an alkyl chain or a cyclic group (e.g., cyclic, heterocyclic, aryl, heteroaryl).

The term “oxo” refers to an oxygen atom (═O), which forms a carbonyl when attached to carbon, an N-oxide when attached to nitrogen, and a sulfoxide or sulfone when attached to sulfur.

The term “thioalkyl” as used herein refers to an —S(alkyl) group, where the point of attachment is through the sulfur atom and the alkyl group is as defined above. The term “thiono” refers to a sulfur atom (═S), which forms a thioketone when attached to carbon.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms.

The term “substituent” refers to a group “substituted” on a moiety described herein. Any atom on any substituent can be substituted. Substituents can include any substituents described herein. Exemplary substituents include, without limitation, alkyl (e.g., C₁, C₂, C₃, C₄, C₅, C₆, C₇, C₈, C₉, C₁₀, C₁₁, C₁₂ straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkyl amino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙalkyl (where n is 0-2), S(O)ₙaryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

The term “pharmaceutically acceptable carrier or adjuvant” refers to a carrier or adjuvant that may be administered to a subject, together with a compound of this invention, and which does not destroy the pharmacological activity thereof and is nontoxic when administered in doses sufficient to deliver a therapeutic amount of the compound.

As used herein, the term “treat” or “treatment” is defined as the application or administration of a compound, alone or in combination with, a second compound to a subject, e.g., a subject, or application or administration of the compound to an isolated tissue or cell, e.g., cell line, from a subject, e.g., a subject, who has a disorder (e.g., a disorder as described herein), a symptom of a disorder, or a predisposition toward a disorder, with the purpose to cure, heal, alleviate, relieve, alter, remedy, ameliorate, improve or affect the disorder, one or more symptoms of the disorder or the predisposition toward the disorder (e.g., to prevent at least one symptom of the disorder or to delay onset of at least one symptom of the disorder).

As used herein, an amount of a compound effective to treat a disorder, or a “therapeutically effective amount” refers to an amount of the compound which is effective, upon single or multiple dose administration to a subject, in treating a cell, or in curing, alleviating, relieving or improving a subject with a disorder beyond that expected in the absence of such treatment.

As used herein, an amount of a compound effective to prevent a disorder, or a “a prophylactically effective amount” of the compound refers to an amount effective, upon single- or multiple-dose administration to the subject, in preventing or delaying the occurrence of the onset or recurrence of a disorder or a symptom of the disorder.

As used herein, the term “subject” is intended to include human and non-human animals. Exemplary human subjects include a human subject having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, dog, cat, cow, pig, etc.

### Compounds

Described herein are compounds of Formula I:

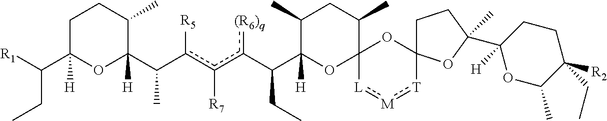

wherein R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano; R₂ is —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; L-M-T together form a structure selected from —C(R₃)ₙ—C(R₃)ₙ—C(R₃)ₙ—, —CR₃═CR₃—C(R₃)ₙ—, and —C(R₃)ₙ—CR₃═CR₃—; or L-M, M-T, or L-M-T, and one to three additional —C(R₃)ₙ—, —O—, —NR₁₁—, or —S— to which they are bound, together form a 3-6 membered cyclyl, heterocyclyl, aryl, or heteroaryl ring, wherein n is independently 1 or 2; each R₃ is independently H, halo, oxo, —OR₁₀, —SR₁₀, —OC(O)R₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl; R₅ is H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁'S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; R₆ is independently H, oxo, —OR_{R}), —SR₁₀, —COR₁₀, —CNR₁₁R₁₂, —C(O)R₁₀, —C(O)OR₁₀, —C(O)NR₁₁R₁₂, —OC(O)R₁₀, —OS(O)R₁₀, —OC(O)OR₁₀, —OS(O)OR₁₀, —NR₁₁R₁₂, ═NR₁₁, —OC(O)NR₁₁R₁₂, —SC(O)NR₁₁R₁₂, halo (e.g., F, Cl, Br, I), —NH₂, cyano, C₁-C₈ alkyl, cyclylalkyl, or aryl; R₅ and R₆ together may optionally form a substituted or unsubstituted 5-8 membered cyclyl, heterocyclyl, aryl, or heteroaryl ring; R₇ is H, halo, C₁-C₈ alkyl, or C₁-C₈ heteroalkyl; R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain; R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR₁₃, —C(O)OR₁₃, —OC(O)R₁₃, —C(O)R₁₃, —S(O)R₁₃, —S(O₂)R₁₃, —NR₁₃R₁₄, cyano, or an amino acid side chain; R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano; R₁₅ and R₁₆ are each independently ═O, ═S, —OR₁₇, —SR₁₇, or —NR₁₇R₁₈, provided R₁₅ and R₁₆ are not both double-bonded moieties; R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl; R₁₉ is —O—, —S—, —NR₁₇—, —N(OH)—, or —N(OR₁₀)—; and q is 1 or 2; provided that when R₁ is —C(O)OH, n is 2, q is 1, R₆ is oxo, and R₇ is methyl, R₂, R₃, and R₅ are not all hydroxy; provided that when R₁ is —C(O)OH, R₆ is oxo, R₇ is methyl, and R₃ and R₅ are hydroxy, R₂ is not benzoxy or benzyloxy; and provided that when R₁ is —C(O)OH, R₆ is oxo, R₇ is methyl, and R₂ is hydroxyl, R₃ or R₅ are not —OCH₂Cl, —OCH₂Br, or —OC(O)CH₂Cl.

Also described herein are compounds of Formula II:

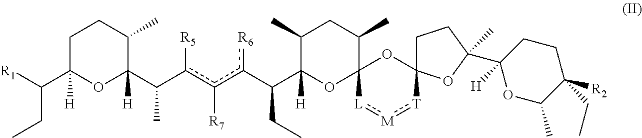

wherein R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano; R₂ is —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; L-M-T together form a structure selected from —C(R₃)₂—C(R₃)₂—C(R₃)₂—, —CR₃═CR₃—C(R₃)₂—, and —C(R₃)₂—CR₃═CR₃—; or L-M, M-T, or L-M-T, and one to three additional —C(R₃)₂—, —O—, —NR₁₁—, or —S— to which they are bound, together form a 3-6 membered cyclyl, heterocyclyl, aryl, or heteroaryl ring; each R₃ is independently H, halo, oxo, —OR₁₀, —SR₁₀, —OC(O)R₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl; R₅ is H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; R₆ is H, oxo, —OR₁₀, —SR₁₀, —OC(O)R₁₀, —OS(O)R₁₀, —OC(O)OR₁₀, —OS(O)OR₁₀, —NR₁₁R₁₂, ═NR₁₁, —OC(O)NR₁₁R₁₂, —SC(O)NR₁₁R₁₂, halo (e.g., F, Cl, Br, I), —NH₂, cyano, cyclylalkyl, or aryl; R₅ and R₆ together may optionally form a substituted or unsubstituted 5-8 membered cyclyl, heterocyclyl, aryl, or heteroaryl ring; R₇ is H, halo, C₁-C₈ alkyl, or C₁-C₈ heteroalkyl; R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain; R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR₁₃, —C(O)OR₁₃, —OC(O)R₁₃, —C(O)R₁₃, —S(O)R₁₃, —S(O₂)R₁₃, —NR₁₃R₁₄, cyano, or an amino acid side chain; R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano; R₁₅ and R₁₆ are each independently ═O, ═S, —OR₁₇, —SR₁₇, or —NR₁₇R₁₈, provided R₁₅ and R₁₆ are not both double-bonded moieties; R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl; and R₁₉ is —O—, —S—, —NR₁₇—, —N(OH)—, or —N(OR₁₀)—; provided that when R₁ is —C(O)OH, R₆ is oxo, and R₇ is methyl, R₂, R₃, and R₅ are not all hydroxy; provided that when R₁ is —C(O)OH, R₆ is oxo, R₇ is methyl, and R₃ and R₅ are hydroxy, R₂ is not benzoxy or benzyloxy; and provided that when R₁ is —C(O)OH, R₆ is oxo, R₇ is methyl, and R₂ is hydroxyl, R₃ or R₅ are not —OCH₂Cl, —OCH₂Br, or —OC(O)CH₂Cl.

Also described herein are compounds of Formula II:

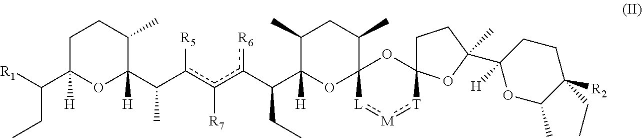

wherein R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano; R₂ is —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; L-M-T together form a structure selected from —C(R₃)₂—C(R₃)₂—C(R₃)₂— and —C(R₃)₂—CR₃═CR₃—; or L-M, M-T, or L-M-T, and one to three additional —C(R₃)₂—, —O—, —NR₁₁—, or —S— to which they are bound, together form a 3-6 membered cyclyl, heterocyclyl, aryl, or heteroaryl ring; each R₃ is independently H, halo, oxo, —OR₁₀, —SR₁₀, —OC(O)R₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl; R₅ is H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; R₆ is independently H, oxo, —OR₁₀, —SR₁₀, —COR₁₀, —CNR₁₁R₁₂, —C(O)R₁₀, —C(O)OR₁₀, —C(O)NR₁₁R₁₂, —OC(O)R₁₀, —OS(O)R₁₀, —OC(O)OR₁₀, —OS(O)OR₁₀, —NR₁₁R₁₂, ═NR₁₁, —OC(O)NR₁₁R₁₂, —SC(O)NR₁₁R₁₂, halo (e.g., F, Cl, Br, I), —NH₂, cyano, C₁-C₈ alkyl, cyclylalkyl, or aryl; R₅ and R₆ together may optionally form a substituted or unsubstituted 5-8 membered cyclyl, heterocyclyl, aryl, or heteroaryl ring; R₇ is H, halo, C₁-C₈ alkyl, or C₁-C₈ heteroalkyl; R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain; R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR_{D}, —C(O)OR₁₃, —OC(O)R₁₃, —C(O)R₁₃, —S(O)R₁₃, —S(O₂)R₁₃, —NR₁₃R₁₄, cyano, or an amino acid side chain; R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano; R₁₅ and R₁₆ are each independently ═O, ═S, —OR₁₇, —SR₁₇, or —NR₁₇R₁₈ provided R₁₅ and R₁₆ are not both double-bonded moieties; R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl; and R₁₉ is —O—, —S—, —NR₁₇—, —N(OH)—, or —N(OR₁₀)—.

Also described herein are compounds of Formula II:

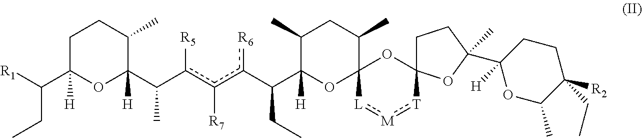

wherein, R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano; R₂ is —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; L-M-T together form a structure selected from —C(R₃)₂—C(R₃)₂—C(R₃)₂—, —CR₃═CR₃—C(R₃)₂—, and —C(R₃)₂—CR₃═CR₃—; or L-M, M-T, or L-M-T, and one to three additional —C(R₃)₂—, —O—, —NR₁₁—, or —S— to which they are bound, together form a 3-6 membered cyclyl, heterocyclyl, aryl, or heteroaryl ring; each R₃ is independently H, halo, oxo, —SR₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl; R₅ is H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁'S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; R₆ is H, oxo, —OR₁₀, —SR₁₀, —COR₁₀, —CNR₁₁R₁₂, —C(O)R₁₀, —C(O)OR₁₀, —C(O)NR₁₁R₁₂, —OC(O)R₁₀, —OS(O)R₁₀, —OC(O)OR₁₀, —OS(O)OR₁₀, —NR₁₁R₁₂, ═NR₁₁, —OC(O)NR₁₁R₁₂, —SC(O)NR₁₁R₁₂, halo (e.g., F, Cl, Br, I), —NH₂, cyano, C₁-C₈ alkyl, cyclylalkyl, or aryl; R₅ and R₆ together may optionally form a substituted or unsubstituted 5-8 membered cyclyl, heterocyclyl, aryl, or heteroaryl ring; R₇ is H, halo, C₁-C₈ alkyl, or C₁-C₈ heteroalkyl; R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain; R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR₁₃, —C(O)OR₁₃, —OC(O)R₁₃, —C(O)R₁₃, —S(O)R₁₃, —S(O₂)R₁₃, —NR₁₃R₁₄, cyano, or an amino acid side chain; R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano; R₁₅ and R₁₆ are each independently ═O, ═S, —OR₁₇, —SR₁₇, or —NR₁₇R₁₈ provided R₁₅ and R₁₆ are not both double-bonded moieties; R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl; and R₁₉ is —O—, —S—, —NR₁₇—, —N(OH)—, or —N(OR₁₀)—.

Also described herein are compounds of the formula below:

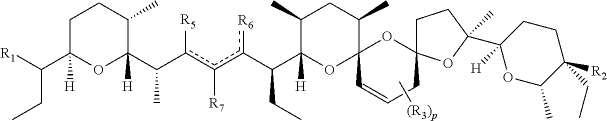

wherein R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano; R₂ is —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; each R₃ is independently H, halo, oxo, —OR₁₀, —SR₁₀, —OC(O)R₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl; R₅ is H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁'S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; R₆ is independently H, oxo, —OR₁₀, —SR₁₀, —COR₁₀, —CNR₁₁R₁₂, —C(O)R₁₀, —C(O)OR₁₀, —C(O)NR₁₁R₁₂, —OC(O)R₁₀, —OS(O)R₁₀, —OC(O)OR₁₀, —OS(O)OR₁₀, —NR₁₁R₁₂, ═NR₁₁, —OC(O)NR₁₁R₁₂, —SC(O)NR₁₁R₁₂, halo (e.g., F, Cl, Br, I), —NH₂, cyano, C₁-C₈ alkyl, cyclylalkyl, or aryl; R₅ and R₆ together may optionally form a substituted or unsubstituted 5-8 membered cyclyl, heterocyclyl, aryl, or heteroaryl ring; R₇ is H, halo, C₁-C₈ alkyl, or C₁-C₈ heteroalkyl; p is 0 to 4; R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain; R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR_{D}, —C(O)OR₁₃, —OC(O)R₁₃, —C(O)R₁₃, —S(O)R₁₃, —S(O₂)R₁₃, —NR₁₃R₁₄, cyano, or an amino acid side chain; R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano; R₁₅ and R₁₆ are each independently ═O, ═S, —OR₁₇, —SR₁₇, or —NR₁₇R₁₈, provided R₁₅ and R₁₆ are not both double-bonded moieties; R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl; and R₁₉ is —O—, —S—, —NR₁₇—, —N(OH)—, or —N(OR₁₀)—; provided that when R₁ is —C(O)OH, R₆ is oxo, and R₇ is methyl, R₂, R₃, and R₅ are not all hydroxy; provided that when R₁ is —C(O)OH, R₆ is oxo, R₇ is methyl, and R₃ and R₅ are hydroxy, R₂ is not benzoxy or benzyloxy; and provided that when R₁ is —C(O)OH, R₆ is oxo, R₇ is methyl, and R₂ is hydroxyl, R₃ or R₅ are not —OCH₂Cl, —OCH₂Br, or —OC(O)CH₂Cl.

Also described herein are compounds of the formula below:

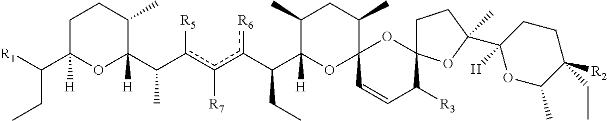

wherein R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano; R₂ is —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; each R₃ is independently H, halo, oxo, —OR₁₀, —SR₁₀, —OC(O)R₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl; R₅ is H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; R₆ is independently H, oxo, —OR₁₀, —SR₁₀, —COR₁₀, —CNR₁₁R₁₂, —C(O)R₁₀, —C(O)OR₁₀, —C(O)NR₁₁R₁₂, —OC(O)R₁₀, —OS(O)R₁₀, —OC(O)OR₁₀, —OS(O)OR₁₀, —NR₁₁R₁₂, ═NR₁₁, —OC(O)NR₁₁R₁₂, —SC(O)NR₁₁R₁₂, halo (e.g., F, Cl, Br, I), —NH₂, cyano, C₁-C₈ alkyl, cyclylalkyl, or aryl; R₅ and R₆ together may optionally form a substituted or unsubstituted 5-8 membered cyclyl, heterocyclyl, aryl, or heteroaryl ring; R₇ is H, halo, C₁-C₈ alkyl, or C₁-C₈ heteroalkyl; R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain; R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR₁₃, —C(O)OR₁₃, —OC(O)R₁₃, —C(O)R₁₃, —S(O)R₁₃, —S(O₂)R₁₃, —NR₁₃R₁₄, cyano, or an amino acid side chain; R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano; R₁₅ and R₁₆ are each independently ═O, ═S, —OR₁₇, —SR₁₇, or —NR₁₇R₁₈, provided R₁₅ and R₁₆ are not both double-bonded moieties; R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl; and R₁₉ is —O—, —S—, —NR₁₇—, —N(OH)—, or —N(OR₁₀)—; provided that when R₁ is —C(O)OH, R₆ is oxo, and R₇ is methyl, R₂, R₃, and R₅ are not all hydroxy; provided that when R₁ is —C(O)OH, R₆ is oxo, R₇ is methyl, and R₃ and R₅ are hydroxy, R₂ is not benzoxy or benzyloxy; and provided that when R₁ is —C(O)OH, R₆ is oxo, R₇ is methyl, and R₂ is hydroxyl, R₃ or R₅ are not —OCH₂Cl, —OCH₂Br, or —OC(O)CH₂Cl.

Also described herein are compounds of the formula below:

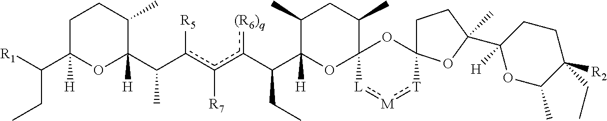

wherein R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano; R₂ is —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; L-M-T together form a structure selected from —C(R₃)ₙ—C(R₃)ₙ—C(R₃)ₙ—, —CR₃═CR₃—C(R₃)ₙ—, and —C(R₃)ₙ—CR₃═CR₃—; or L-M, M-T, or L-M-T, and one to three additional —C(R₃)ₙ—, —O—, —NR₁₁—, or —S— to which they are bound, together form a 3-6 membered cyclyl, heterocyclyl, aryl, or heteroaryl ring, wherein n is independently 1 or 2; each R₃ is independently H, halo, oxo, —OR₁₀, —SR₁₀, —OC(O)R₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁'S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl; R₅ is H, halo, oxo, —OR_{R}), —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀; R₆ is independently H, oxo, —OR_{R}), —SR₁₀, —COR₁₀, —CNR₁₁R₁₂, —C(O)R₁₀, —C(O)OR₁₀, —C(O)NR₁₁R₁₂, —OC(O)R₁₀, —OS(O)R₁₀, —OC(O)OR₁₀, —OS(O)OR₁₀, —NR₁₁R₁₂, ═NR₁₁, —OC(O)NR₁₁R₁₂, —SC(O)NR₁₁R₁₂, halo (e.g., F, Cl, Br, I), —NH₂, cyano, C₁-C₈ alkyl, cyclylalkyl, or aryl; R₅ and R₆ together may optionally form a substituted or unsubstituted 5-8 membered cyclyl, heterocyclyl, aryl, or heteroaryl ring; R₇ is H, halo, C₁-C₈ alkyl, or C₁-C₈ heteroalkyl; R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain; R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR₁₃, —C(O)OR₁₃, —OC(O)R₁₃, —C(O)R₁₃, —S(O)R₁₃, —S(O₂)R₁₃, —NR₁₃R₁₄, cyano, or an amino acid side chain; R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano; R₁₅ and R₁₆ are each independently ═O, ═S, —OR₁₇, —SR₁₇, or —NR₁₇R₁₈, provided R₁₅ and R₁₆ are not both double-bonded moieties; R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl; R₁₉ is —O—, —S—, —NR₁₇—, —N(OH)—, or —N(OR₁₀)—; and q is 1 or 2; provided that when R₁ is —C(O)OH, n is 2, q is 1, R₆ is oxo, and R₇ is methyl, R₂, R₃, and R₅ are not all hydroxy; provided that when R₁ is —C(O)OH, R₆ is oxo, R₇ is methyl, and R₃ and R₅ are hydroxy, R₂ is not benzoxy or benzyloxy; and provided that when R₁ is —C(O)OH, R₆ is oxo, R₇ is methyl, and R₂ is hydroxyl, R₃ or R₅ are not —OCH₂Cl, —OCH₂Br, or —OC(O)CH₂Cl.

Compounds described and claimed herein include, but are not limited to, the compounds disclosed in Table 1.

TABLE 1
Salinomycin analogs. R′ and R″ are independently C₁-C₆ alkyl, aryl, or arylalkyl.

| COMPOUND | STRUCTURE |
|---|---|
| 1 | 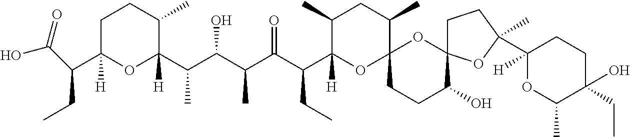 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(CC[C@@H](O)[C@]3(CC[C@@](C)([C@H]4CC[C@](O)(CC)[C@H](C)O4)O3)O2)[C@H](C)C[C@@H]1C] |
| 2 | 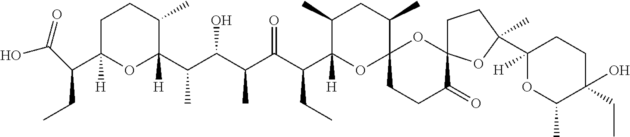 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(CCC(=O)[C@]3(CC[C@@](C)([C@H]4CC[C@](O)(CC)[C@H](C)O4)O3)O2)[C@H](C)C[C@@H]1C] |
| 3 | 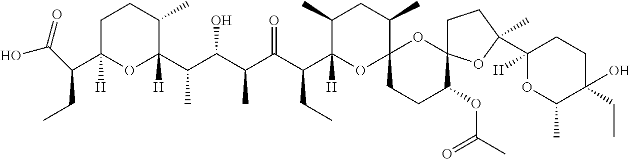 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(CC[C@@H](OC(C)=O)[C@]3(CC[C@@](C)([C@H]4CC[C@](O)(CC)[C@H](C)O4)O3)O2)[C@H](C)C[C@@H]1C] |
| 4 | 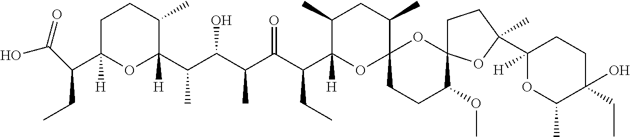 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(CC[C@@H](OC)[C@]3(CC[C@@](C)([C@H]4CC[C@](O)(CC)[C@H](C)O4)O3)O2)[C@H](C)C[C@@H]1C] |
| 5 | 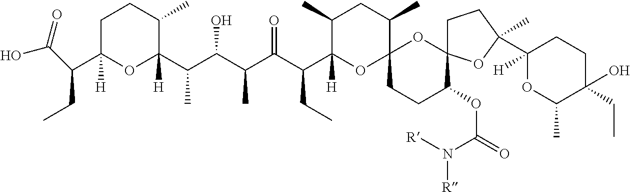 |
| 6 | 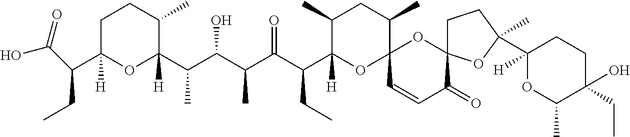 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=CC(=O)[C@]3(CC[C@@](C)([C@H]4CC[C@](O)(CC)[C@H](C)O4)O3)O2)[C@H](C)C[C@@H]1C] |
| 7 | 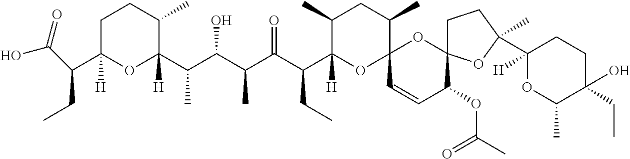 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 8 | 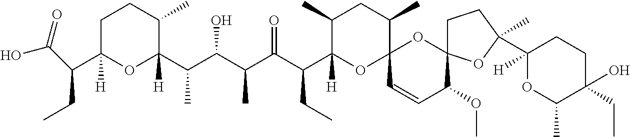 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](OC)[C@]3(CC[C@@](C)([C@H]4CC[C@](O)(CC)[C@H](C)O4)O3)O2)[C@H](C)C[C@@H]1C] |
| 9 | 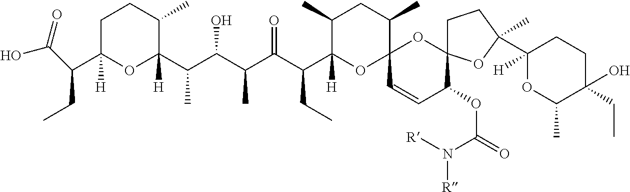 |
| 10 | 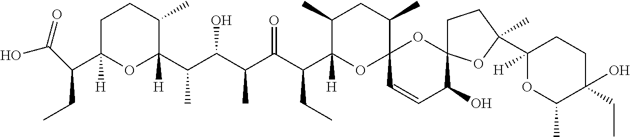 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@H](O)[C@]3(CC[C@@](C)([C@H]4CC[C@](O)(CC)[C@H](C)O4)O3)O2)[C@H](C)C[C@@H]1C] |
| 11 | 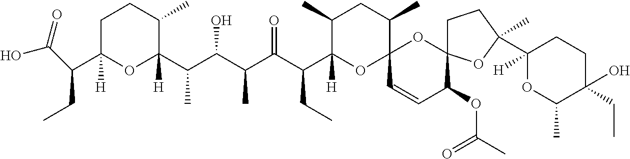 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@H](OC(C)=O)[C@]3(CC[C@@](C)([C@H]4CC[C@](O)(CC)[C@H](C)O4)O3)O2)[C@H](C)C[C@@H]1C] |
| 12 | 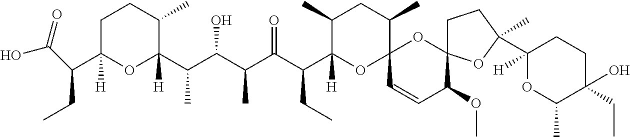 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@H](OC)[C@]3(CC[C@@](C)([C@H]4CC[C@](O)(CC)[C@H](C)O4)O3)O2)[C@H](C)C[C@@H]1C] |
| 13 | 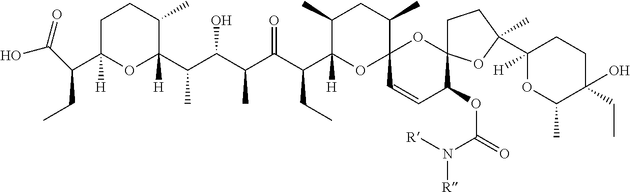 |
| 14 | 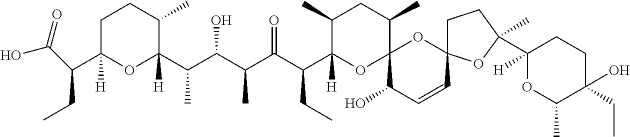 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@@]2(O[C@]3(C=C[C@@H]2O)CC[C@@](C)([C@H]2CC[C@](O)(CC)[C@H](C)O2)O3)[C@H](C)C[C@@H]1C] |
| 15 | 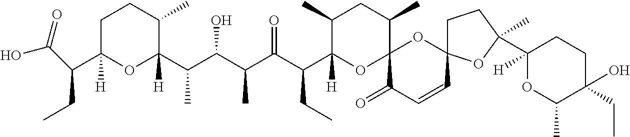 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@@]2(O[C@]3(C=CC2=O)CC[C@@](C)([C@H]2CC[C@](O)(CC)[C@H](C)O2)O3)[C@H](C)C[C@@H]1C] |
| 16 | 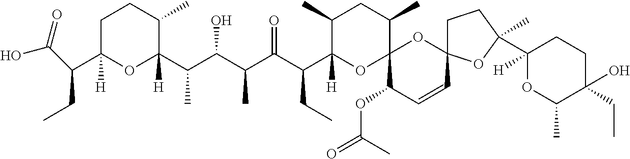 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@@]2(O[C@]3(C=C[C@@H]2OC(C)=O)CC[C@@](C)([C@H]2CC[C@](O)(CC)[C@H](C)O2)O3)[C@H](C)C[C@@H]1C] |
| 17 | 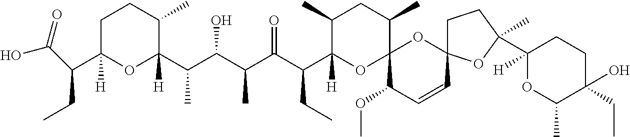 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@@]2(O[C@]3(C=C[C@@H]2OC)CC[C@@](C)([C@H]2CC[C@](O)(CC)[C@H](C)O2)O3)[C@H](C)C[C@@H]1C] |
| 18 | 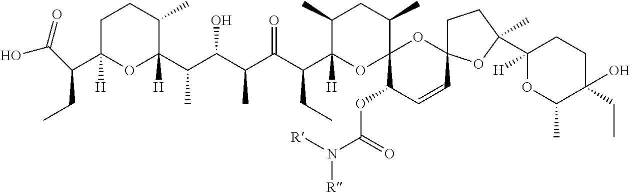 |
| 19 | 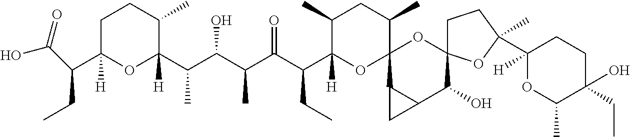 |
| 20 | 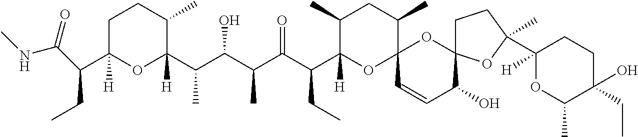 [SMILES: CC[C@@H](C(=O)[C@@H](C)[C@@H](O)[C@H](C)[C@@H]1O[C@@H]([C@@H](CC)C(=O)NC)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 21 | 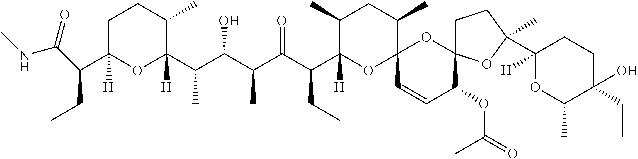 [SMILES: CC[C@@H](C(=O)[C@@H](C)[C@@H](O)[C@H](C)[C@@H]1O[C@@H]([C@@H](CC)C(=O)NC)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 22 | 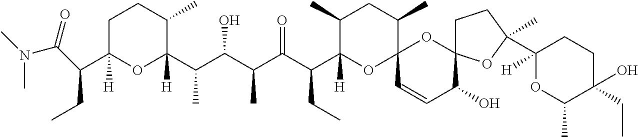 [SMILES: CC[C@@H](C(=O)[C@@H](C)[C@@H](O)[C@H](C)[C@@H]1O[C@@H]([C@@H](CC)C(=O)N(C)C)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 23 | 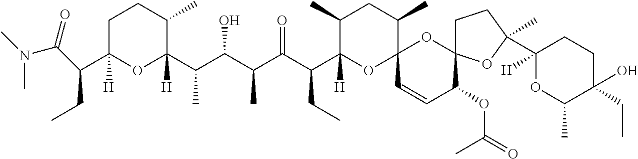 [SMILES: CC[C@@H](C(=O)[C@@H](C)[C@@H](O)[C@H](C)[C@@H]1O[C@@H]([C@@H](CC)C(=O)N(C)C)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 24 | 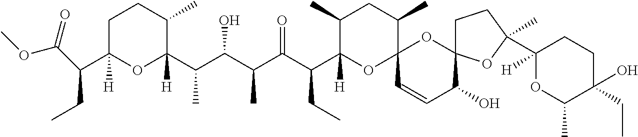 [SMILES: CC[C@@H](C(=O)[C@@H](C)[C@@H](O)[C@H](C)[C@@H]1O[C@@H]([C@@H](CC)C(=O)OC)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 25 | 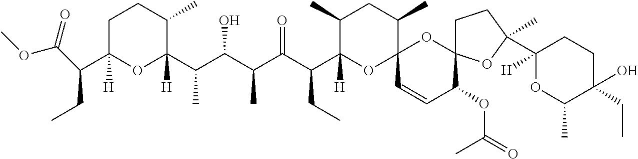 [SMILES: CC[C@@H](C(=O)[C@@H](C)[C@@H](O)[C@H](C)[C@@H]1O[C@@H]([C@@H](CC)C(=O)OC)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 26 | 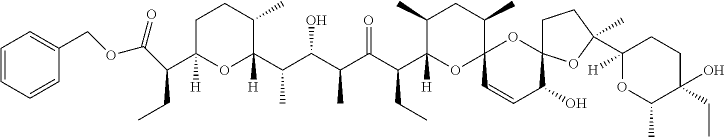 [SMILES: CC[C@@H](C(=O)[C@@H](C)[C@@H](O)[C@H](C)[C@@H]1O[C@@H]([C@@H](CC)C(=O)OCc2ccccc2)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 27 | 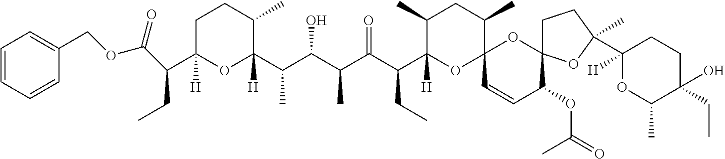 [SMILES: CC[C@@H](C(=O)[C@@H](C)[C@@H](O)[C@H](C)[C@@H]1O[C@@H]([C@@H](CC)C(=O)OCc2ccccc2)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 28 | 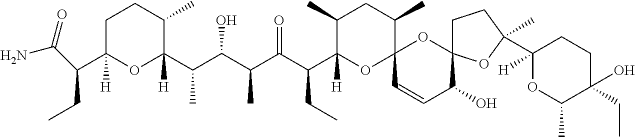 [SMILES: CC[C@@H](C(=O)[C@@H](C)[C@@H](O)[C@H](C)[C@@H]1O[C@@H]([C@@H](CC)C(N)=O)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 29 | 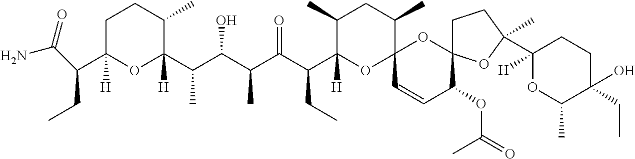 [SMILES: CC[C@@H](C(=O)[C@@H](C)[C@@H](O)[C@H](C)[C@@H]1O[C@@H]([C@@H](CC)C(N)=O)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 30 | 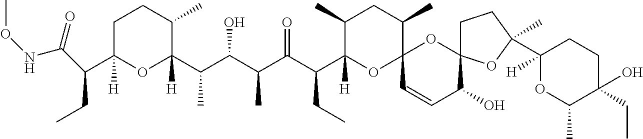 [SMILES: CC[C@@H](C(=O)[C@@H](C)[C@@H](O)[C@H](C)[C@@H]1O[C@@H]([C@@H](CC)C(=O)NOC)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 31 | 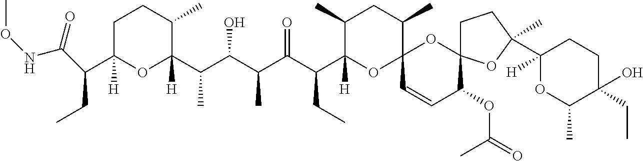 [SMILES: CC[C@@H](C(=O)[C@@H](C)[C@@H](O)[C@H](C)[C@@H]1O[C@@H]([C@@H](CC)C(=O)NOC)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 32 | 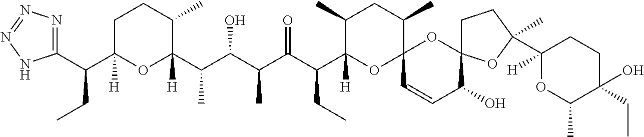 [SMILES: CC[C@@H](C(=O)[C@@H](C)[C@@H](O)[C@H](C)[C@@H]1O[C@@H]([C@@H](CC)c2nnn[nH]2)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 33 | 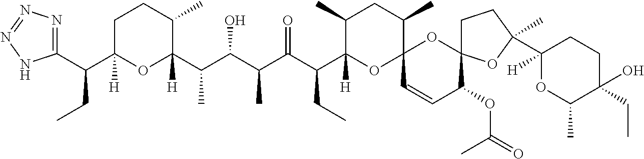 [SMILES: CC[C@@H](C(=O)[C@@H](C)[C@@H](O)[C@H](C)[C@@H]1O[C@@H]([C@@H](CC)c2nnn[nH]2)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 34 | 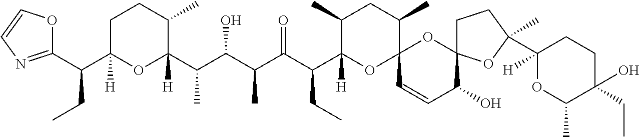 [SMILES: CC[C@@H](C(=O)[C@@H](C)[C@@H](O)[C@H](C)[C@@H]1O[C@@H]([C@@H](CC)c2ncco2)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 35 | 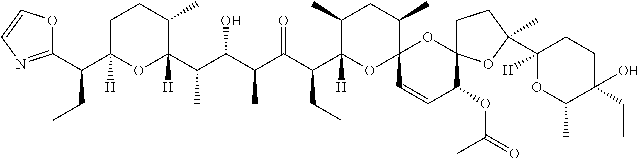 [SMILES: CC[C@@H](C(=O)[C@@H](C)[C@@H](O)[C@H](C)[C@@H]1O[C@@H]([C@@H](CC)c2ncco2)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 36 | 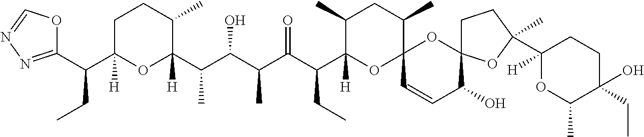 [SMILES: CC[C@@H](C(=O)[C@@H](C)[C@@H](O)[C@H](C)[C@@H]1O[C@@H]([C@@H](CC)c2nnco2)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 37 | 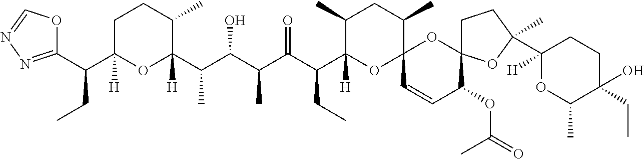 [SMILES: CC[C@@H](C(=O)[C@@H](C)[C@@H](O)[C@H](C)[C@@H]1O[C@@H]([C@@H](CC)c2nnco2)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 38 | 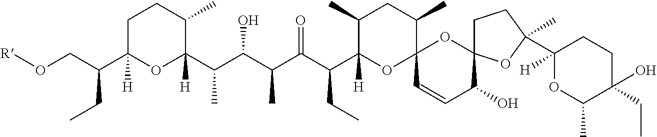 |
| 39 | 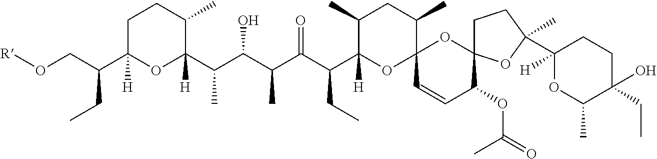 |
| 40 | 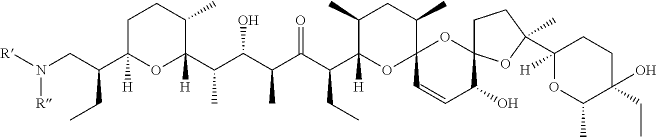 |
| 41 | 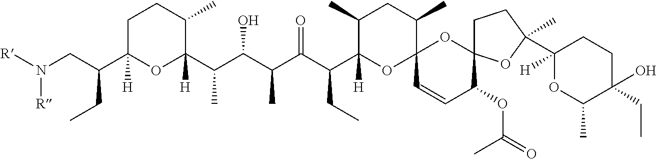 |
| 42 | 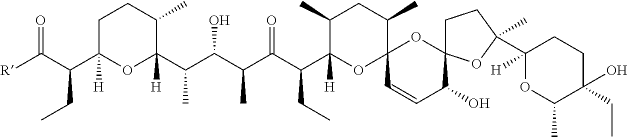 |
| 43 | 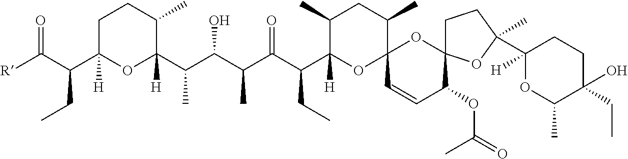 |
| 44 | 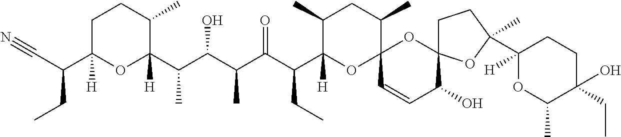 [SMILES: CC[C@@H](C#N)[C@H]1CC[C@H](C)[C@H]([C@@H](C)[C@H](O)[C@H](C)C(=O)[C@H](CC)[C@H]2O[C@]3(C=C[C@@H](O)[C@]4(CC[C@@](C)([C@H]5CC[C@](O)(CC)[C@H](C)O5)O4)O3)[C@H](C)C[C@@H]2C)O1] |
| 45 | 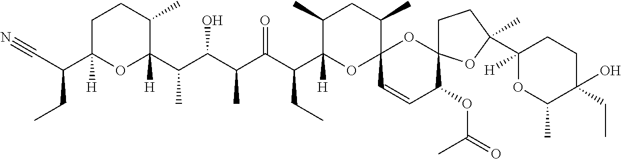 [SMILES: CC[C@@H](C#N)[C@H]1CC[C@H](C)[C@H]([C@@H](C)[C@H](O)[C@H](C)C(=O)[C@H](CC)[C@H]2O[C@]3(C=C[C@@H](OC(C)=O)[C@]4(CC[C@@](C)([C@H]5CC[C@](O)(CC)[C@H](C)O5)O4)O3)[C@H](C)C[C@@H]2C)O1] |
| 46 | 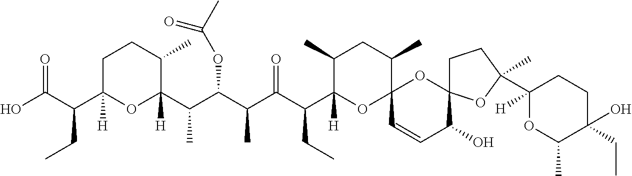 [SMILES: CC[C@@H](C(=O)[C@@H](C)[C@@H](OC(C)=O)[C@H](C)[C@@H]1O[C@@H]([C@@H](CC)C(=O)O)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 47 | 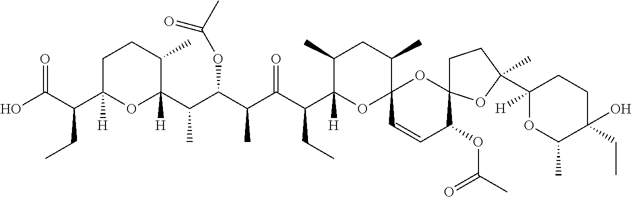 [SMILES: CC[C@@H](C(=O)[C@@H](C)[C@@H](OC(C)=O)[C@H](C)[C@@H]1O[C@@H]([C@@H](CC)C(=O)O)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 48 | 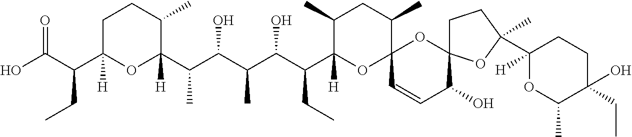 [SMILES: CC[C@@H]([C@@H](O)[C@@H](C)[C@@H](O)[C@H](C)[C@@H]1O[C@@H]([C@@H](CC)C(=O)O)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 49 | 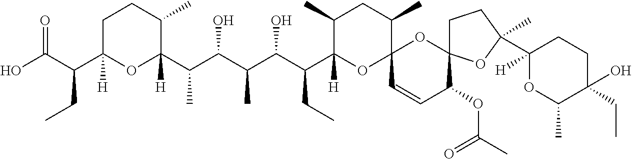 [SMILES: CC[C@@H]([C@@H](O)[C@@H](C)[C@@H](O)[C@H](C)[C@@H]1O[C@@H]([C@@H](CC)C(=O)O)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 50 | 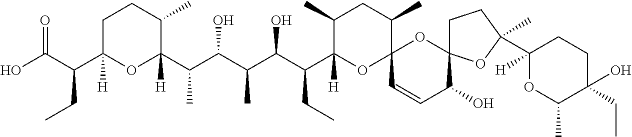 [SMILES: CC[C@@H]([C@H](O)[C@@H](C)[C@@H](O)[C@H](C)[C@@H]1O[C@@H]([C@@H](CC)C(=O)O)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 51 | 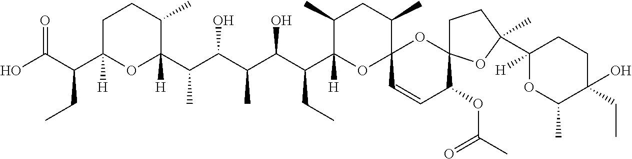 [SMILES: CC[C@@H]([C@H](O)[C@@H](C)[C@@H](O)[C@H](C)[C@@H]1O[C@@H]([C@@H](CC)C(=O)O)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 52 | 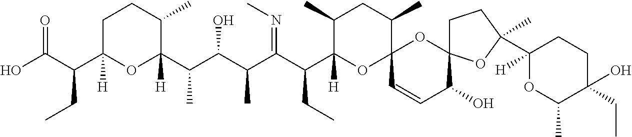 [SMILES: CC[C@@H](C(=O)O)[C@H]1CC[C@H](C)[C@H]([C@@H](C)[C@H](O)[C@H](C)/C(=N\C)[C@H](CC)[C@H]2O[C@]3(C=C[C@@H](O)[C@]4(CC[C@@](C)([C@H]5CC[C@](O)(CC)[C@H](C)O5)O4)O3)[C@H](C)C[C@@H]2C)O1] |
| 53 | 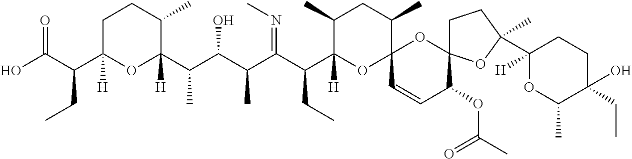 [SMILES: CC[C@@H](C(=O)O)[C@H]1CC[C@H](C)[C@H]([C@@H](C)[C@H](O)[C@H](C)/C(=N\C)[C@H](CC)[C@H]2O[C@]3(C=C[C@@H](OC(C)=O)[C@]4(CC[C@@](C)([C@H]5CC[C@](O)(CC)[C@H](C)O5)O4)O3)[C@H](C)C[C@@H]2C)O1] |
| 54 | 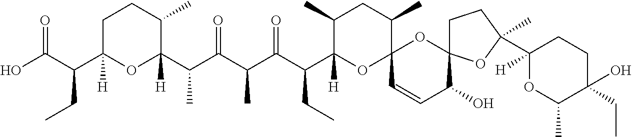 [SMILES: CC[C@@H](C(=O)[C@@H](C)C(=O)[C@H](C)[C@@H]1O[C@@H]([C@@H](CC)C(=O)O)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 55 | 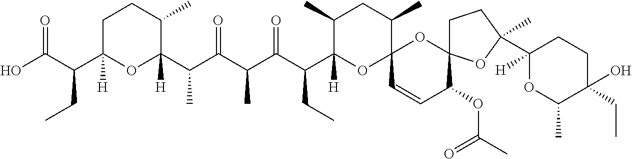 [SMILES: CC[C@@H](C(=O)[C@@H](C)C(=O)[C@H](C)[C@@H]1O[C@@H]([C@@H](CC)C(=O)O)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 56 | 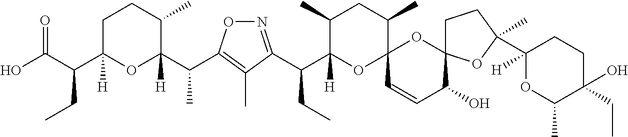 [SMILES: CC[C@@H](C(=O)O)[C@H]1CC[C@H](C)[C@H]([C@@H](C)c2onc([C@H](CC)[C@H]3O[C@]4(C=C[C@@H](O)[C@]5(CC[C@@](C)([C@H]6CC[C@](O)(CC)[C@H](C)O6)O5)O4)[C@H](C)C[C@@H]3C)c2C)O1] |
| 57 | 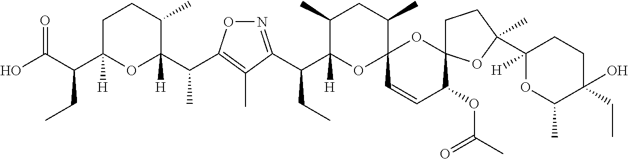 [SMILES: CC[C@@H](C(=O)O)[C@H]1CC[C@H](C)[C@H]([C@@H](C)c2onc([C@H](CC)[C@H]3O[C@]4(C=C[C@@H](OC(C)=O)[C@]5(CC[C@@](C)([C@H]6CC[C@](O)(CC)[C@H](C)O6)O5)O4)[C@H](C)C[C@@H]3C)c2C)O1] |
| 58 | 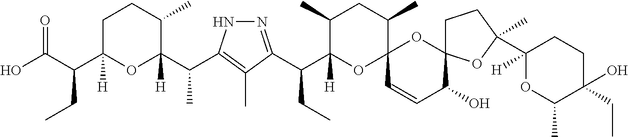 [SMILES: CC[C@@H](C(=O)O)[C@H]1CC[C@H](C)[C@H]([C@@H](C)c2[nH]nc([C@H](CC)[C@H]3O[C@]4(C=C[C@@H](O)[C@]5(CC[C@@](C)([C@H]6CC[C@](O)(CC)[C@H](C)O6)O5)O4)[C@H](C)C[C@@H]3C)c2C)O1] |
| 59 | 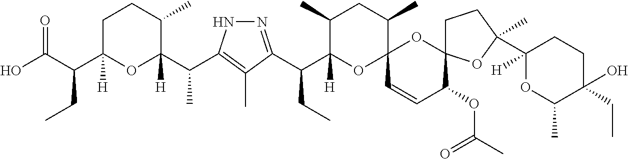 [SMILES: CC[C@@H](C(=O)O)[C@H]1CC[C@H](C)[C@H]([C@@H](C)c2[nH]nc([C@H](CC)[C@H]3O[C@]4(C=C[C@@H](OC(C)=O)[C@]5(CC[C@@](C)([C@H]6CC[C@](O)(CC)[C@H](C)O6)O5)O4)[C@H](C)C[C@@H]3C)c2C)O1] |
| 60 | 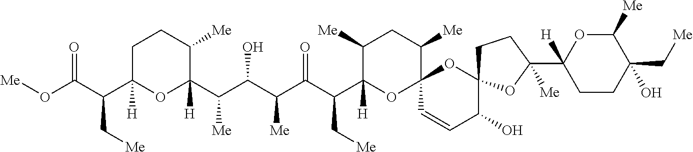 |
| 61 | 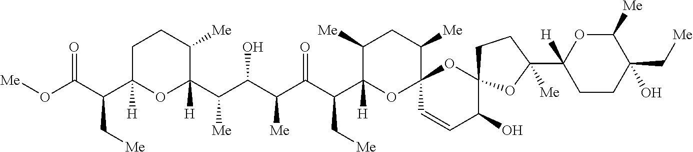 |
| 62 | 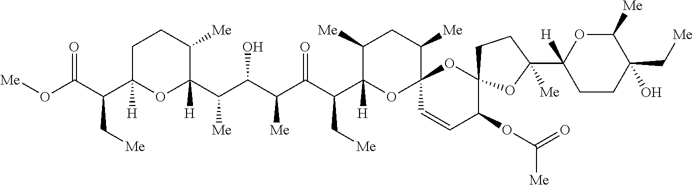 |
| 63 | 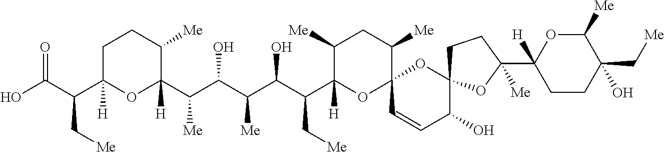 |
| 64 | 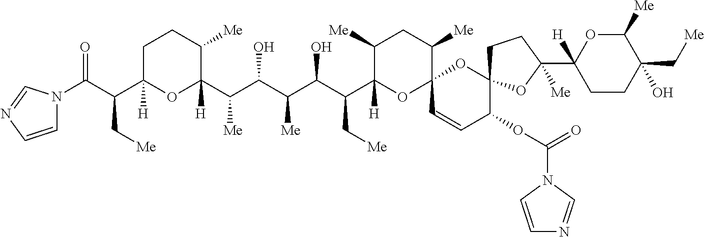 |
| 65 | 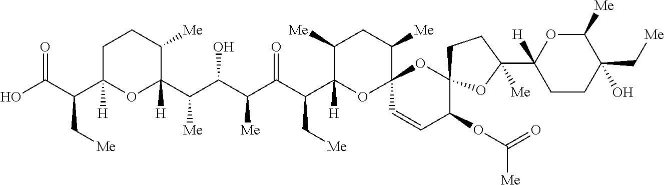 |
| 66 | 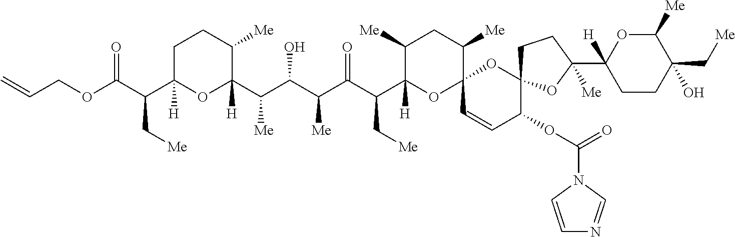 |
| 67 | 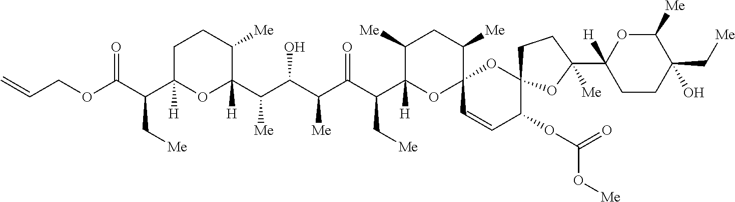 [SMILES: C=CCOC(=O)[C@H](CC)[C@H]1CC[C@H](C)[C@H]([C@@H](C)[C@H](O)[C@H](C)C(=O)[C@H](CC)[C@H]2O[C@]3(C=C[C@@H](OC(=O)OC)[C@]4(CC[C@@](C)([C@H]5CC[C@](O)(CC)[C@H](C)O5)O4)O3)[C@H](C)C[C@@H]2C)O1] |
| 68 | 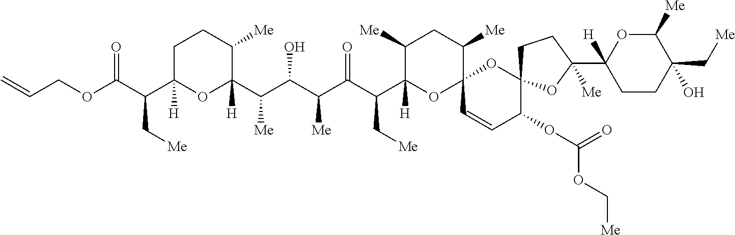 [SMILES: C=CCOC(=O)[C@H](CC)[C@H]1CC[C@H](C)[C@H]([C@@H](C)[C@H](O)[C@H](C)C(=O)[C@H](CC)[C@H]2O[C@]3(C=C[C@@H](OC(=O)OCC)[C@]4(CC[C@@](C)([C@H]5CC[C@](O)(CC)[C@H](C)O5)O4)O3)[C@H](C)C[C@@H]2C)O1] |
| 69 | 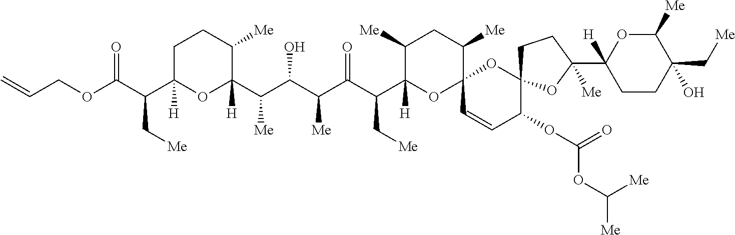 [SMILES: C=CCOC(=O)[C@H](CC)[C@H]1CC[C@H](C)[C@H]([C@@H](C)[C@H](O)[C@H](C)C(=O)[C@H](CC)[C@H]2O[C@]3(C=C[C@@H](OC(=O)OC(C)C)[C@]4(CC[C@@](C)([C@H]5CC[C@](O)(CC)[C@H](C)O5)O4)O3)[C@H](C)C[C@@H]2C)O1] |
| 70 | 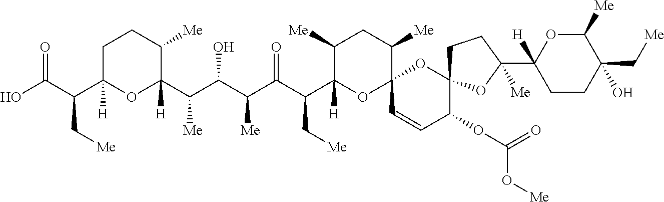 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](OC(=O)OC)[C@]3(CC[C@@](C)([C@H]4CC[C@](O)(CC)[C@H](C)O4)O3)O2)[C@H](C)C[C@@H]1C] |
| 71 | 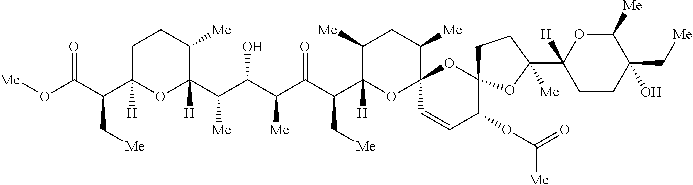 [SMILES: CC[C@@H](C(=O)[C@@H](C)[C@@H](O)[C@H](C)[C@@H]1O[C@@H]([C@@H](CC)C(=O)OC)CC[C@@H]1C)[C@H]1O[C@@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 72 | 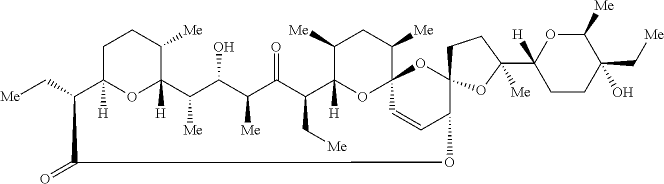 |
| 73 | 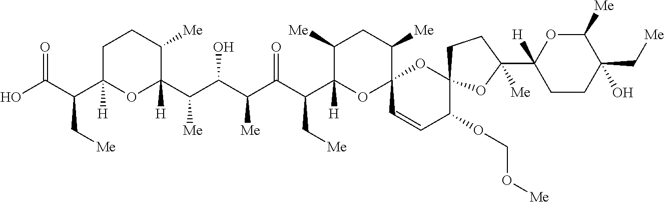 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](OCOC)[C@]3(CC[C@@](C)([C@H]4CC[C@](O)(CC)[C@H](C)O4)O3)O2)[C@H](C)C[C@@H]1C] |
| 74 | 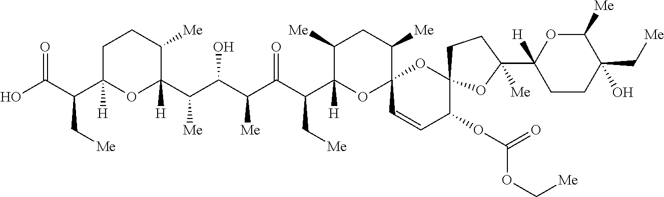 [SMILES: CCOC(=O)O[C@@H]1C=C[C@]2(O[C@H]([C@@H](CC)C(=O)[C@@H](C)[C@@H](O)[C@H](C)[C@@H]3O[C@@H]([C@@H](CC)C(=O)O)CC[C@@H]3C)[C@@H](C)C[C@H]2C)O[C@@]12CC[C@@](C)([C@H]1CC[C@](O)(CC)[C@H](C)O1)O2] |
| 75 | 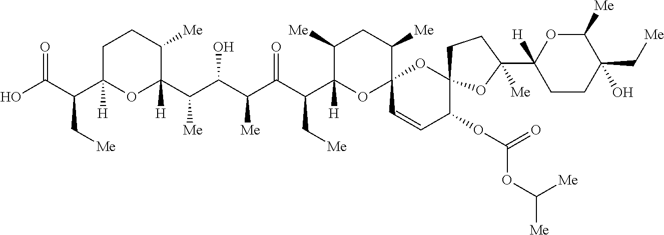 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](OC(=O)OC(C)C)[C@]3(CC[C@@](C)([C@H]4CC[C@](O)(CC)[C@H](C)O4)O3)O2)[C@H](C)C[C@@H]1C] |
| 76 | 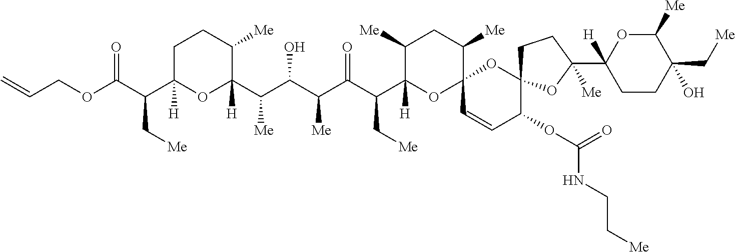 |
| 77 | 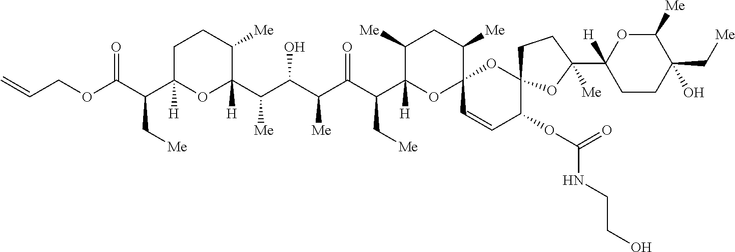 [SMILES: C=CCOC(=O)[C@H](CC)[C@H]1CC[C@H](C)[C@H]([C@@H](C)[C@H](O)[C@H](C)C(=O)[C@H](CC)[C@H]2O[C@]3(C=C[C@@H](OC(=O)NCCO)[C@]4(CC[C@@](C)([C@H]5CC[C@](O)(CC)[C@H](C)O5)O4)O3)[C@H](C)C[C@@H]2C)O1] |
| 78 | 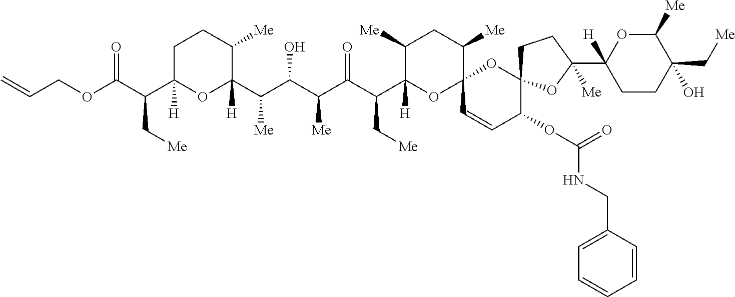 [SMILES: C=CCOC(=O)[C@H](CC)[C@H]1CC[C@H](C)[C@H]([C@@H](C)[C@H](O)[C@H](C)C(=O)[C@H](CC)[C@H]2O[C@]3(C=C[C@@H](OC(=O)NCc4ccccc4)[C@]4(CC[C@@](C)([C@H]5CC[C@](O)(CC)[C@H](C)O5)O4)O3)[C@H](C)C[C@@H]2C)O1] |
| 79 | 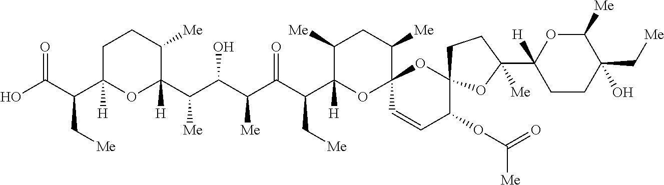 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@@]2(C=C[C@@H](OC(C)=O)[C@]3(CC[C@@](C)([C@H]4CC[C@](O)(CC)[C@H](C)O4)O3)O2)[C@H](C)C[C@@H]1C] |
| 80 | 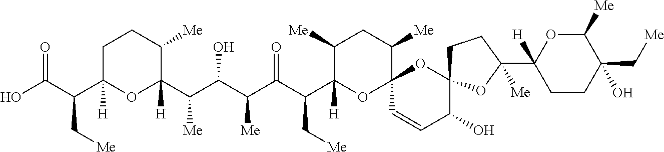 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@@]2(C=C[C@@H](O)[C@]3(CC[C@@](C)([C@H]4CC[C@](O)(CC)[C@H](C)O4)O3)O2)[C@H](C)C[C@@H]1C] |
| 81 | 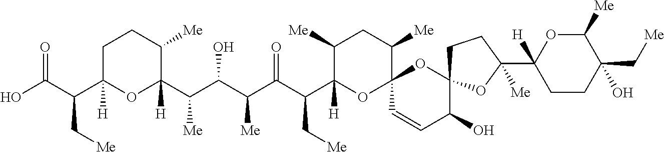 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@@]2(C=C[C@H](O)[C@]3(CC[C@@](C)([C@H]4CC[C@](O)(CC)[C@H](C)O4)O3)O2)[C@H](C)C[C@@H]1C] |
| 82 | 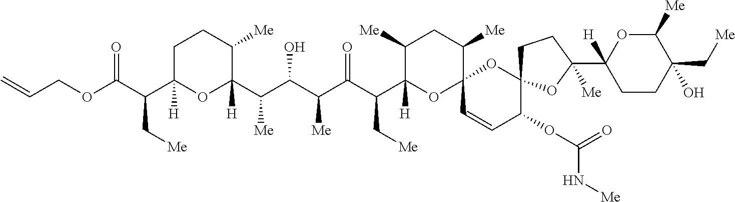 [SMILES: C=CCOC(=O)[C@H](CC)[C@H]1CC[C@H](C)[C@H]([C@@H](C)[C@H](O)[C@H](C)C(=O)[C@H](CC)[C@H]2O[C@]3(C=C[C@@H](OC(=O)NC)[C@]4(CC[C@@](C)([C@H]5CC[C@](O)(CC)[C@H](C)O5)O4)O3)[C@H](C)C[C@@H]2C)O1] |
| 83 | 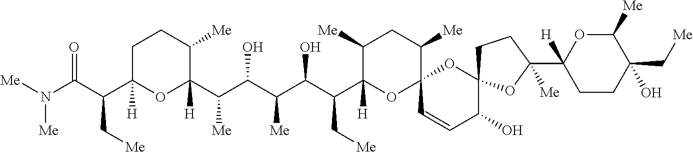 [SMILES: CC[C@@H]([C@H](O)[C@@H](C)[C@@H](O)[C@H](C)[C@@H]1O[C@@H]([C@@H](CC)C(=O)N(C)C)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 84 | 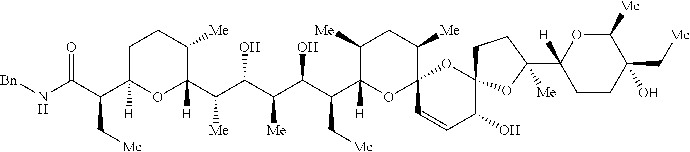 [SMILES: CC[C@@H]([C@H](O)[C@@H](C)[C@@H](O)[C@H](C)[C@@H]1O[C@@H]([C@@H](CC)C(=O)NCc2ccccc2)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 85 | 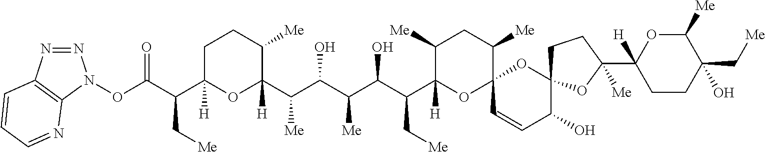 [SMILES: CC[C@@H]([C@H](O)[C@@H](C)[C@@H](O)[C@H](C)[C@@H]1O[C@@H]([C@@H](CC)C(=O)On2nnc3cccnc32)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C] |
| 86 | 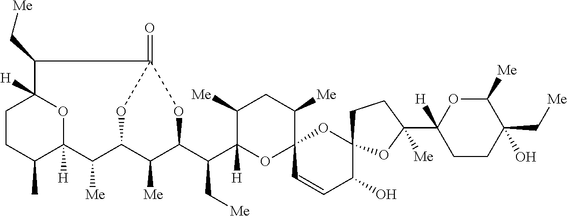 |
| 87 | 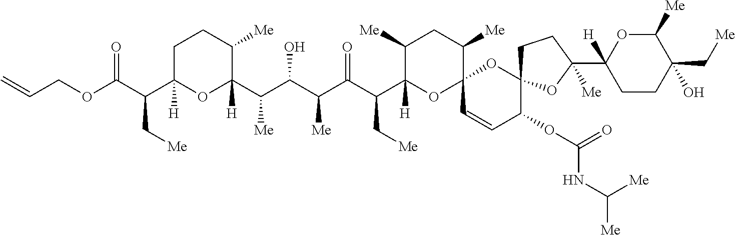 [SMILES: C=CCOC(=O)[C@H](CC)[C@H]1CC[C@H](C)[C@H]([C@@H](C)[C@H](O)[C@H](C)C(=O)[C@H](CC)[C@H]2O[C@]3(C=C[C@@H](OC(=O)NC(C)C)[C@]4(CC[C@@](C)([C@H]5CC[C@](O)(CC)[C@H](C)O5)O4)O3)[C@H](C)C[C@@H]2C)O1] |
| 88 | 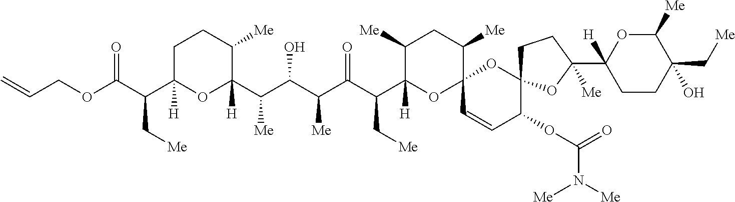 |
| 89 | 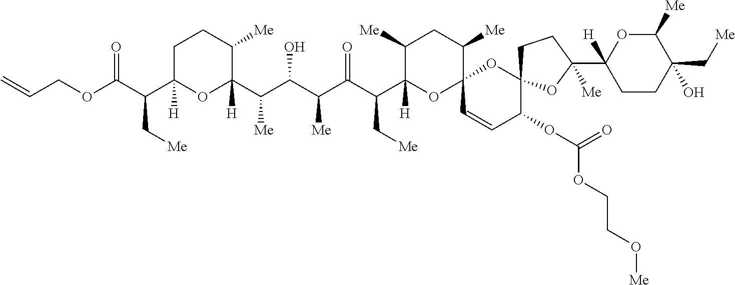 |
| 90 | 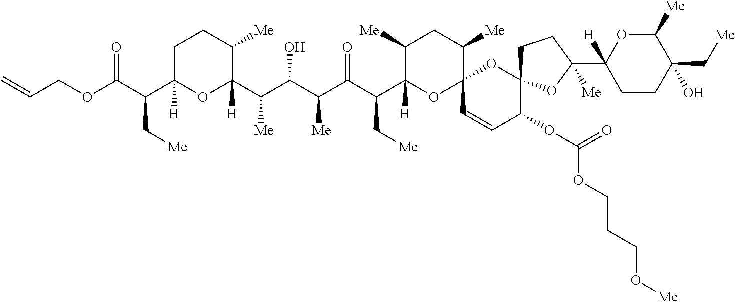 |
| 91 | 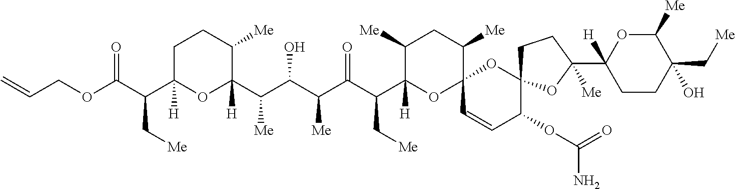 |
| 92 | 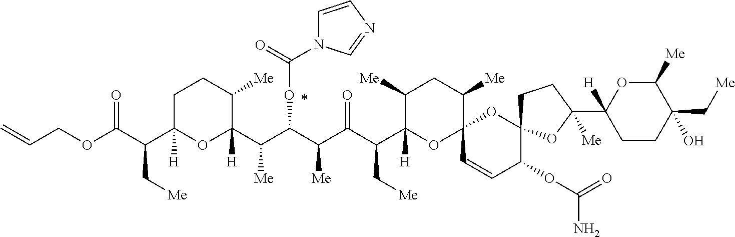 |
| 93 | 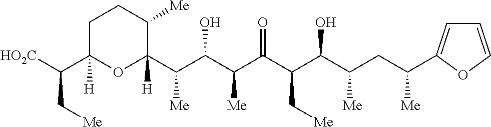 |
| 94 | 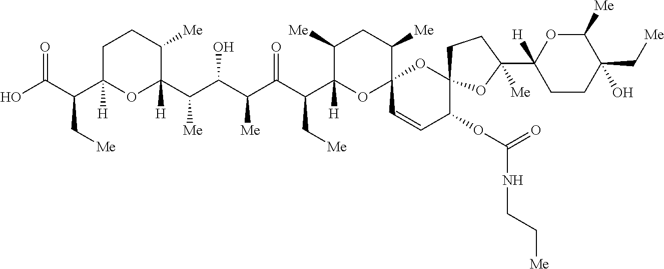 |
| 95 | 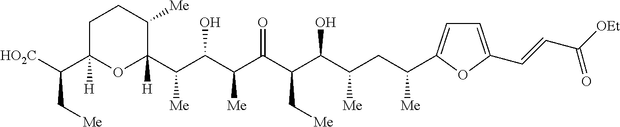 [SMILES: CCOC(=O)/C=C/c1ccc([C@H](C)C[C@H](C)[C@H](O)[C@@H](CC)C(=O)[C@@H](C)[C@@H](O)[C@H](C)[C@@H]2O[C@@H]([C@@H](CC)C(=O)O)CC[C@@H]2C)o1] |
| 96 | 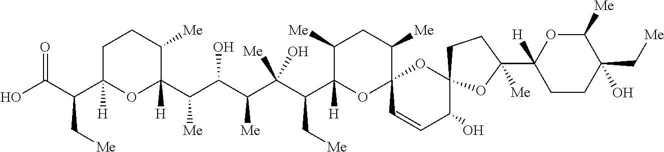 |
| 97 | 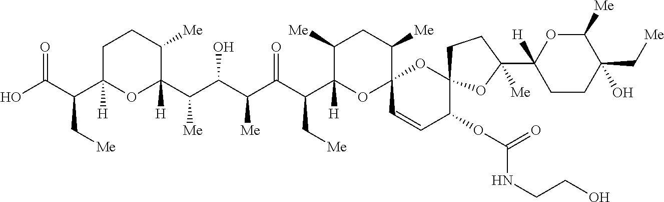 |
| 98 | 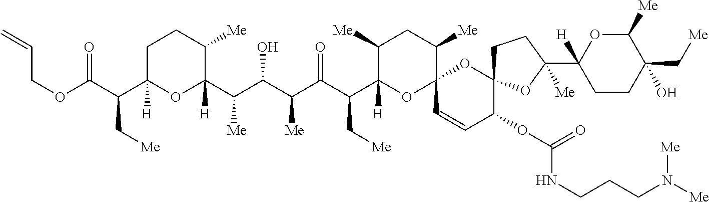 |
| 99 | 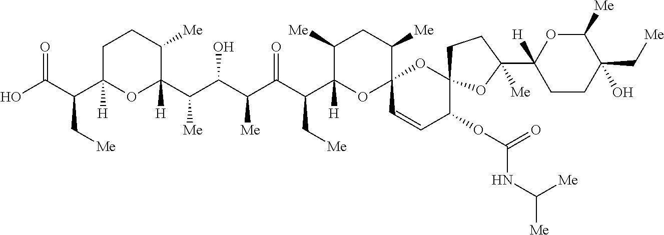 |
| 100 | 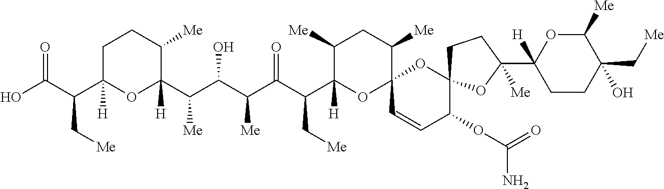 |
| 101 | 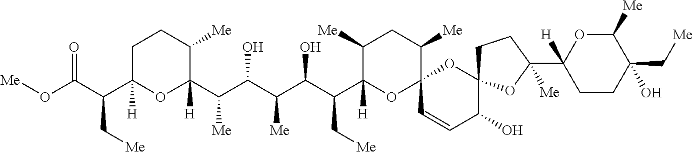 |
| 102 | 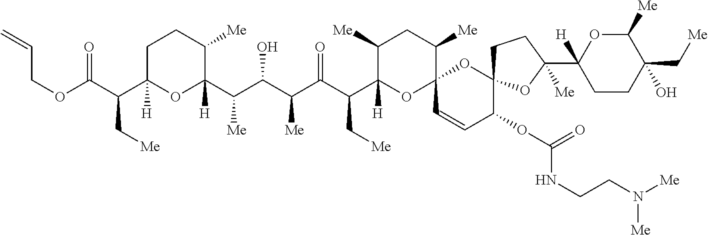 |
| 103 | 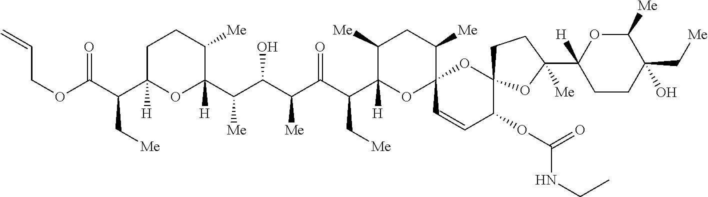 |
| 104 | 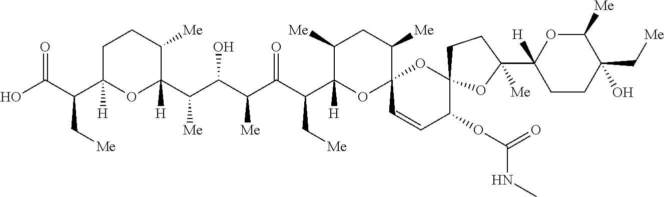 |
| 105 | 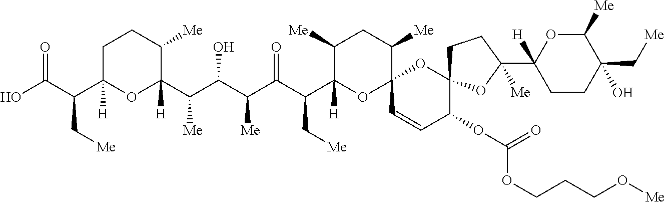 |
| 106 | 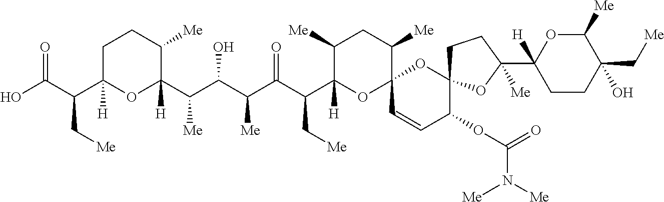 |
| 107 | 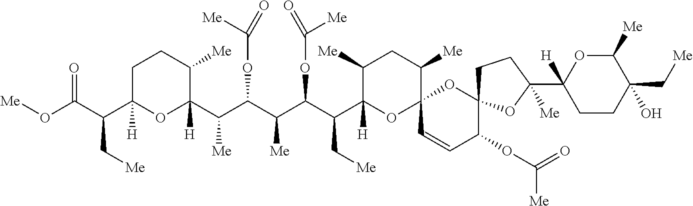 |
| 108 | 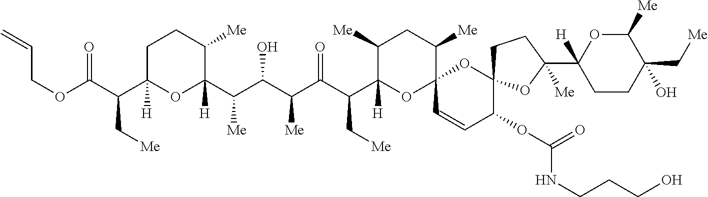 |
| 109 | 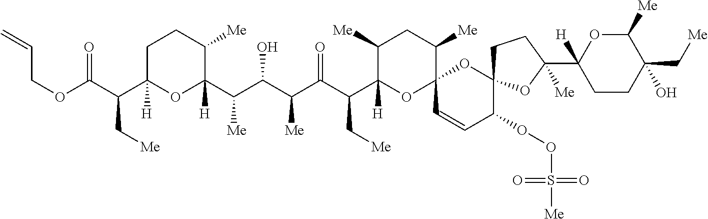 |
| 110 | 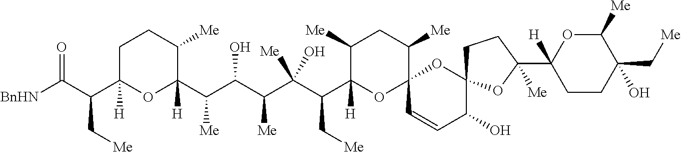 |
| 111 | 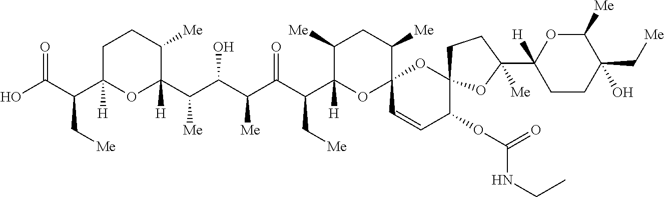 |
| 112 | 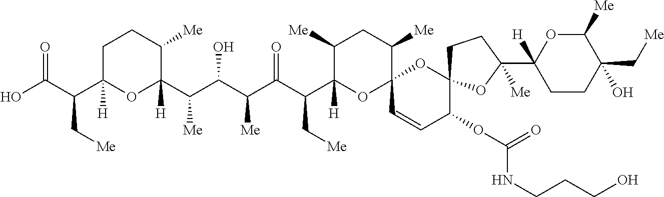 |
| 113 | 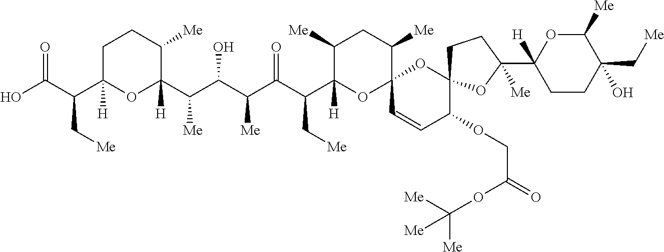 |
| 114 | 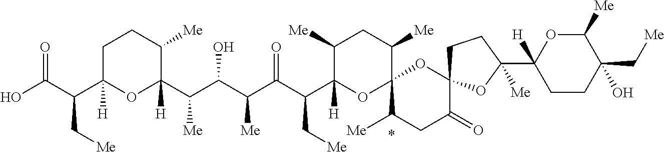 |
| 115 | 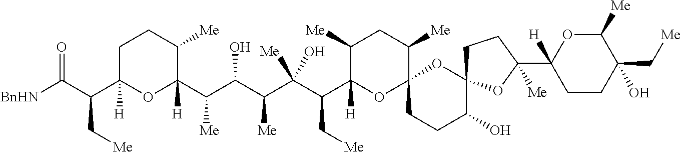 |
| 116 | 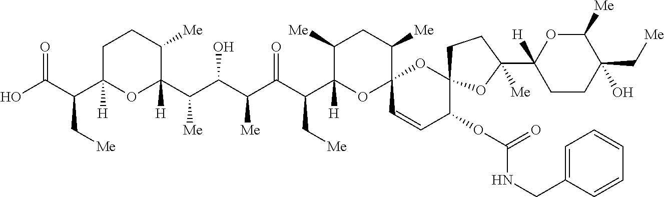 |
| 117 | 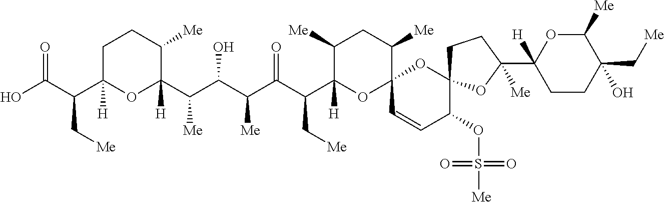 |
| 118 | 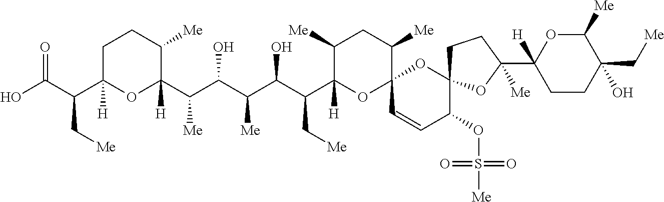 |

Compounds of the invention, e.g., compounds disclosed in Table 1, may have binding activity against proteins and other targets, such as e-cadherin (ECad), Twist, or GFP expressed by mammory epithelial cells (HMLE). In some embodiments, a compound as described herein, e.g., compounds disclosed in Table 1, is active in the ALDEFLUOR assay (e.g., as described herein).

The compounds described herein can be made using a variety of synthetic techniques. In some embodiments, the starting material will be salinomycin or a salinomycin salt, e.g., salinomycin sodium. Crude salinomycin or salinomycin salt may be commercially purchased (e.g., from Zhejiang Shenghua Baike Pharmaceutical, China) and further purified (e.g., with preparative chromatography) prior to being modified. Salinomycin is a natural product that can be purified from bacteria such as Streptomyces albus. The structure of salinomycin is shown below:

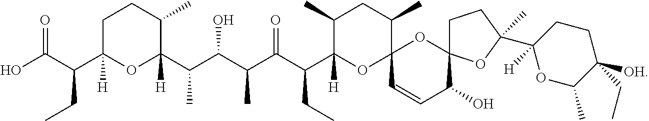 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C]

As can be appreciated by the skilled artisan, methods of synthesizing the compounds of the formula (I) will be evident to those of ordinary skill in the art. In some embodiments, one or more hydroxyl groups of salinomycin may be removed, oxidized, acetylated, or aminated. In some embodiments, the terminal carboxylic acid may be oxidized, reduced, aminated amidated, esterified, silated, thiolated, or protected. In some embodiments a keto and a nearby hydroxyl may be cyclized, heterocyclized, reduced, aminated, or aminated. Additionally, the various synthetic steps may be performed in an alternate sequence or order to give the desired compounds. One or more reactive sites may be protected or deprotected, as needed to give the desired compounds. Additionally synthetic details are provided in the Examples (below).

Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds disclosed herein typically contain one or more asymmetric centers and thus may occur as racemates and racemic mixtures, single enantiomers, individual diastereomers and diastereomeric mixtures. Where a structure is shown with a specific stereochemistry at an atomic center, the stereochemistry is intended to remain fixed at that atomic center, however when an atomic center is shown without stereochemistry, it is contemplated that the disclosed compound includes compounds with all possible stereochemistry at that atomic center, e.g., both R and S or both (+) and (−). The compounds of this invention may also contain linkages (e.g., carbon-carbon bonds) or substituents that can restrict bond rotation, e.g. restriction resulting from the presence of a ring or double bond. Accordingly, all cis/trans and E/Z isomers are expressly included in the present invention.

The compounds disclosed herein may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The compounds of this invention include the compounds themselves, as well as their salts and their prodrugs. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds. The compounds of the invention additionally comprise hydrates of the compounds and hydrates of their salts. Hydrates are complexes of the compounds containing one or more water molecules.

The compounds of this invention may be modified by appending appropriate functionalities to enhance selected biological properties, e.g., targeting to a particular tissue. Such modifications are known in the art and include those which increase biological penetration into a given biological compartment (e.g., blood, lymphatic system, central nervous system), increase oral availability, increase solubility to allow administration by injection, alter metabolism and alter rate of excretion.

In an alternate embodiment, the compounds described herein may be used as platforms or scaffolds that may be utilized in combinatorial chemistry techniques for preparation of derivatives and/or chemical libraries of compounds. Such derivatives and libraries of compounds have biological activity and are useful for identifying and designing compounds possessing a particular activity. Combinatorial techniques suitable for utilizing the compounds described herein are known in the art as exemplified by Obrecht, D. and Villalgrodo, J. M., Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, A. W., Curr. Opin. Chem. Bio., (1997) 1, 60. Thus, one embodiment relates to a method of using the compounds described herein for generating derivatives or chemical libraries comprising: 1) providing a body comprising a plurality of wells; 2) providing one or more compounds identified by methods described herein in each well; 3) providing an additional one or more chemicals in each well; 4) isolating the resulting one or more products from each well. An alternate embodiment relates to a method of using the compounds described herein for generating derivatives or chemical libraries comprising: 1) providing one or more compounds described herein attached to a solid support; 2) treating the one or more compounds identified by methods described herein attached to a solid support with one or more additional chemicals; 3) isolating the resulting one or more products from the solid support. In the methods described above, “tags” or identifier or labeling moieties may be attached to and/or detached from the compounds described herein or their derivatives, to facilitate tracking, identification or isolation of the desired products or their intermediates. Such moieties are known in the art. The chemicals used in the aforementioned methods may include, for example, solvents, reagents, catalysts, protecting group and deprotecting group reagents and the like. Examples of such chemicals are those that appear in the various synthetic and protecting group chemistry texts and treatises referenced herein.

### Compositions and Routes of Administration

The compounds described herein may be administered to a subject as a pharmaceutically acceptable composition or a dosage form. In some cases the compositions or dosage forms may be in the form of a kit containing the composition or dosage form along with instructions for administering the compound. The kit may additionally comprise a diluent and instructions for administering the diluents along with the compound as intended (e.g., as a composition or dosage form). The pharmaceutically acceptable compositions or dosage forms may be administered along with additional therapeutic agents, if present, in amounts effective for achieving a modulation of disease or disease symptoms, including those described herein. The additional therapeutic agents may be administered simultaneous with the compounds described herein, or they may be administered sequentially with the compounds described herein.

In some embodiments, the pharmaceutical acceptable composition additionally comprises a solubilizer and/or emulsifying agent. Exemplary solubilizers and/or emulsifying agents include amphiphilic molecules such as a long-chain amphiphilic molecules. In some embodiments, the amphiphilic molecule is non-ionic. In some embodiment, the solubilizer and/or emulsifying agent comprises a polyalkylene oxide such as PEG. In some embodiments, the solubilizer and/or emulsifying agent is a polysorbate, e.g., a polyoxyethylene derivative of sorbitan monolaurate, e.g., a Tween such as Tween® 80. In some embodiments, the solubilizers and/or emulsifying agents are mixtures of polyethylene glycol and derivatives of hydroxystearate, e.g., mono- and di-esters of 12-hydroxystearic acid, e.g., a solutol such as Solutol® HS 15. In some embodiments, the solubilizers and/or emulsifying agents are polyethoxylated castor oil, e.g., a Cremophor® such as Cremophor® EL. Other solubilizers and/or emulsifying agents that have been recognized as safe by an appropriate regulatory body, e.g., the U.S. Food and Drug Administration (FDA), may also be used.

In some embodiments, the pharmaceutically acceptable composition additionally comprises a miscible organic solvent, e.g., an alcohol, an organic acid, or a polar-organic solvent. In some embodiments, the miscible organic solvent is an alcohol e.g., ethanol or propylene glycol. In some embodiments, the miscible organic solvent is an organic acid, e.g., propanoic acid. In some embodiments, the miscible organic solvent is a polar-organic solvent or polar aprotic solvent, e.g., DMSO.

In some embodiments, the aqueous composition (e.g., a compound or composition) described herein comprises a stabilizer. Exemplary stabilizers include chelating agents, for example, EDTA or EDTA salts, e.g., disodium EDTA, or citric acid. Exemplary stabilizers also include antioxidants, such as ascorbic acid, tocopherol tocopherol derivatives, and metabisulfites, e.g., sodium metabisulfite, as well as preservatives, such as benzyl alcohol, parabens, or cholorobutanol.

In addition to the components described above, the pharmaceutically acceptable compositions can include additional ingredients such as additional pharmaceutically acceptable carriers, adjuvants and vehicles. Exemplary pharmaceutically acceptable carriers, adjuvants and vehicles include ion exchangers, lecithin, self-emulsifying drug delivery systems (SEDDS) such as d-alpha-tocopherol polyethyleneglycol 1000 succinate, emulsifying agents used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, and polyethylene glycol. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

Pharmaceutically acceptable compositions can include diluents, fillers, salts, buffers, stabilizers, solubilizers and other materials which are well-known in the art. The choice of pharmaceutically-acceptable carrier to be used in conjunction with the compounds of the present invention is basically determined by the way the compound is to be administered. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. It will also be understood that the compound can be provided as a pharmaceutically acceptable pro-drug, or an active metabolite can be used. Furthermore it will be appreciated that agents may be modified, e.g., with targeting moieties, moieties that increase their uptake, biological half-life (e.g., pegylation), etc.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical acceptable compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compounds and pharmaceutical compositions described herein may be useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Compounds, compositions and dosage forms may be used for treating, e.g., ameliorating, alleviating, curing, maintaining a cure (i.e., the prevention or delay of relapse) of a disorder (e.g, a tumor), or preventing the spread of a disorder to another part of the subject, e.g., metastasis. Treatment after a disorder has started aims to reduce, ameliorate or altogether eliminate the disorder, and/or its associated symptoms, to prevent it from becoming worse, to slow the rate of progression, or to prevent the disorder from re-occurring once it has been initially eliminated (i.e., to prevent a relapse). A suitable dose and therapeutic regimen may vary depending upon the specific compound used, the mode of delivery of the compound, and whether it is used alone or in combination. As used herein, a therapeutically effective amount is an amount of a compound or composition that inhibits CSC-dependent tumor formation, progression, and/or spread (e.g., metastasis).

A therapeutically effective amount can refer to any one or more of the compounds or compositions described herein, or discovered using the methods described herein, that have inhibitory properties (e.g, inhibit the growth and/or survival of CSCs and/or mesenchymal cells). Methods for establishing a therapeutically effective amount for any compounds or compositions described herein will be known to one of ordinary skill in the art. As used herein, pharmacological compositions comprise compounds or compositions that have therapeutic utility, and a pharmaceutically acceptable carrier, i.e., that facilitate delivery of compounds or compositions, in a therapeutically effective amount. The effective amount for any particular application can also vary depending on such factors as the cancer being treated, the particular compound being administered, the size of the subject, or the severity of the disease or condition. One of ordinary skill in the art can empirically determine the effective amount of a particular molecule of the invention without necessitating undue experimentation. Combined with the teachings provided herein, by choosing among the various active compounds and weighing factors such as potency, relative bioavailability, subject body weight, severity of adverse side-effects and preferred mode of administration, an effective prophylactic or therapeutic treatment regimen can be planned with the goal of avoiding substantial toxicity and yet effective to treat the particular subject. In some embodiments a useful compound increases the average length of survival, increases the average length of progression-free survival, and/or reduces the rate of recurrence, of subjects treated with the compound in a statistically significant manner.

Subject doses of the compounds described herein typically range from about 0.1 μg to 10,000 mg, more typically from about 1 μg to 8000 mg, e.g., from about 10 μg to 100 mg once or more per day, week, month, or other time interval. Stated in terms of subject body weight, typical dosages in certain embodiments of the invention range from about 0.1 μg to 20 mg/kg/day, e.g., from about 1 to 10 mg/kg/day, e.g., from about 1 to 5 mg/kg/day. The absolute amount will depend upon a variety of factors including the concurrent treatment, the number of doses and the individual subject parameters including age, physical condition, size and weight. These are factors well known to those of ordinary skill in the art and can be addressed with no more than routine experimentation. It is often the case that a maximum dose be used, that is, the highest safe dose according to sound medical judgment. The dose used may be the maximal tolerated dose or a sub-therapeutic dose or any dose there between. Multiple doses of the molecules of the invention are also contemplated. When the molecules of the invention are administered in combination a sub-therapeutic dosage of either of the molecules, or a sub-therapeutic dosage of both, may be used in the treatment of a subject having, or at risk of developing, cancer. When the two classes of drugs are used together, the cancer medicament may be administered in a sub-therapeutic dose to produce a desirable therapeutic result. A sub-therapeutic dose is a dosage which is less than that dosage which would produce a therapeutic result in the subject if administered in the absence of the other agent. Thus, the sub-therapeutic dose of a cancer medicament is one which would not produce the desired therapeutic result in the subject in the absence of the administration of the molecules of the invention. Therapeutic doses of cancer medicaments are well known in the field of medicine for the treatment of cancer. These dosages have been extensively described in references such as Remington's Pharmaceutical Sciences, 18th ed., 1990; as well as many other medical references relied upon by the medical profession as guidance for the treatment of cancer.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Subjects may, however, require intermittent treatment on a long-term basis upon any recurrence of disease symptoms.

In some embodiments, the compounds and compositions described herein are incorporated into a dosage form. In some embodiments, the dosage form is a parenteral dosage form, e.g., for administration to a subject intravenously. In some embodiments, the dosage form is composition in a sterile, sealed container (e.g., a bottle, a vial). In some embodiments, the dosage form may be an oral dosage form, e.g., for administration to a subject orally. In some embodiments, an oral dosage form additionally comprises flavors, or fragrances, or both, to modify the taste or odor of the oral dosage form.

### Methods of Treatment

The compounds and compositions described herein can be administered to cells in culture, e.g. in vitro or ex vivo, or to a subject, e.g., in vivo, to treat, prevent, and/or diagnose a variety of disorders, including those described herein below. The compounds and compostions described herein may inhibit the proliferation of cancer stem cells and/or mesenchymal cells.

### Neoplastic Disorders

Compounds and compositions described herein may be used for treating, e.g., ameliorating, alleviating, curing, maintaining a cure (i.e., delaying relapse) of a proliferative disorder, such as cancer. A “proliferative disorder” is a disease or disorder characterized by cells that have the capacity for autonomous growth or replication, e.g., an abnormal state or condition characterized by proliferative cell growth. Exemplary proliferative disorders include solid tumors and cancers of the blood, for example, carcinoma, sarcoma, metastatic disorders (e.g., tumors arising from prostate, colon, lung, breast and liver origin), hematopoietic proliferative disorders, e.g., leukemias, metastatic tumors. Prevalent cancers include: breast, prostate, colon, lung, liver, and pancreatic cancers. Treatment with the aqueous composition may be in an amount effective to ameliorate at least one symptom of the proliferative disorder, e.g., reduced cell proliferation, reduced tumor mass, etc.

The disclosed methods are useful in the treatment of cancer, including for example, solid tumors, soft tissue tumors, and metastases thereof. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine.

Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primaiy; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS—Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated or prevented in accordance with the methods described herein.

In some embodiments, the cancer is, or is characterized as comprising or enriched for, cancer stem cells (CSCs), tumor-initiating cells, mesenchymal cells, or mesenchymal-like cells associated with cancer, or mesenchymal cancerous cells. For example, the compounds or compositions can be administered to a subject to kill, inhibit the growth of, limit the proliferation of, or cause other beneficial changes to a subject (e.g., a human) having cancer. In some embodiments, the cancer is associated with CSCs, or tumor-initiating cells, mesenchymal cells, or mesenchymal-like cells associated with cancer, or mesenchymal cancerous cells, or the cancer is characterized as being enriched with CSCs and/or mesenchymal cells (e.g, a CSC-enriched tumor, a tumor with mesenchymal cells, or a tumor with cells that have undergone an epithelial-to-mesenchymal transition). In an embodiment, treatment with a compound or composition described herein may reduce the spread of cancer, e.g., a metastatic cancer. In an embodiment, treatment with a compound or composition described herein may reduce the likelihood of relapse of a cancer, e.g., reducing the likelihood of self-initiation of a new tumor. In embodiments where treatment has started after a diagnosis with a disorder, the compounds and compositions described herein can reduce, ameliorate or altogether eliminate the disorder, and/or its associated symptoms, to keep it from becoming worse, to slow the rate of progression, or to minimize the rate of recurrence of the disorder once it has been initially eliminated (i.e., to avoid a relapse). A suitable dose and therapeutic regimen may vary depending upon the specific composition used, the mode of delivery of the compound, and whether it is used alone or in combination. As used herein, a therapeutically effective amount is an amount of a compound or composition that inhibits cancer (e.g., a CSC-enriched tumor, a tumor with mesenchymal cells, or a tumor with cells that have undergone an epithelial-to-mesenchymal transition) formation, progression, and/or spread (e.g., metastasis). A therapeutically effective amount can refer to any one or more of the compounds or compositions described herein, or discovered using the methods described herein, that have CSC-enriched tumor inhibitory properties (e.g, inhibit the growth and/or survival of CSCs, or cancerous mesenchymal cells). The effective amount of a compound or composition described herein can vary depending on such factors as the cancer being treated, the size of the subject, or the severity and/or progression of the disease or condition. In some embodiments a useful composition increases the average length of survival, increases the average length of progression-free survival, and/or reduces the rate of recurrence, of subjects treated with the aqueous composition in a statistically significant manner. In some embodiments a compound or composition described herein is used to inhibit the growth or differentiation of a cancer stem cell or cancerous mesenchymal cell, e.g., by contacting the cancer stem cell or cancerous mesenchymal cell with a compound or composition described herein. The contacting may take place in vitro or in vivo. In some embodiments, the cancer stem cell or cancerous mesenchymal cell has or is characterized by activity in a transcription factor selected from Snail1, Snail2, Goosecoid, FoxC1, FoxC2, TWIST, E2A, SIP-1/Zeb-2, dEF1/Zeb1, LEF1, Myc, HMGA2, TAZ, Klf8, HIF-1, HOXB7, SIM2s, and Fos. In some embodiments, the cancer stem cell or cancerous mesenchymal cell has or is characterized by activity in a pathway selected from TGF-β, Wnt, BMP, Notch, HGF-Met, EGF, IGF, PDGF, FGF, P38-mapk, Ras, PB Kinase-Akt, Src, and NF-kB. In some embodiments, the compounds and compositions described herein can be administered to cells in culture, e.g. in vitro or ex vivo, or to a subject, e.g., in vivo, to treat, modulate, and/or diagnose a variety of disorders, including those described herein below.

### Cancer Combination Therapies

In certain embodiments, the compounds and compositions described herein may be taken alone or in combination with other therapeutics. In one embodiment, a mixture of two or more compositions may be administered to a subject in need thereof. In yet another embodiment, one or more compositions may be administered with one or more therapeutic agents for the treatment or avoidance of various diseases, including, for example, cancer, diabetes, neurodegenerative diseases, cardiovascular disease, blood clotting, inflammation, flushing, obesity, aging, stress, etc. In various embodiments, combination therapies comprising a compound or composition described herein may refer to (1) pharmaceutical compositions that comprise one or more compositions in combination with one or more therapeutic agents (e.g., one or more therapeutic agents described herein); and (2) co-administration of one or more compounds or compositions described herein with one or more therapeutic agents wherein the compounds or compositions and therapeutic agent have not been formulated in the same compositions (but may be present within the same kit or package, such as a blister pack or other multi-chamber package; connected, separately sealed containers (e.g., foil pouches) that can be separated by the user; or a kit where the compounds or composition and other therapeutic agent(s) are in separate vessels). When using separate compositions, the compounds or compositions described herein may be administered at the same time as, intermittently, staggered, prior to, subsequent to, or combinations thereof, with respect to the administration of another therapeutic agent.

In some embodiments, a compound or composition described herein is administered together with an additional cancer treatment. Exemplary cancer treatments include, for example: chemotherapy, antibiotics, targeted therapies such as antibody therapies, immunotherapy, and hormonal therapy. For example, nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, antibiotics, enzymes, epipodophyllotoxins, platinum coordination complexes, vinca alkaloids, substituted ureas, methyl hydrazine derivatives, adrenocortical suppressants, hormone antagonists, enzyme inhibitors, endostatin, taxols, camptothecins, doxorubicins and their analogs, and combinations thereof.

Examples of each of these treatments are provided below.

Chemotherapy

In some embodiments, a compound or composition described herein is administered with a chemotherapy agent. Chemotherapy is the treatment of cancer with drugs that can destroy cancer cells. “Chemotherapy” usually refers to cytotoxic drugs which affect rapidly dividing cells in general, in contrast with targeted therapy. Chemotherapy drugs interfere with cell division in various possible ways, e.g., with the duplication of DNA or the separation of newly formed chromosomes. Most forms of chemotherapy target all rapidly dividing cells and are not specific for cancer cells, although some degree of specificity may come from the inability of many cancer cells to repair DNA damage, while normal cells generally can.

Examples of chemotherapeutic agents used in cancer therapy include, for example, alkylating and alkylating-like agents such as nitrogen mustards (e.g., chlorambucil, chlormethine, cyclophosphamide, ifosfamide, and melphalan), nitrosoureas (e.g., carmustine, fotemustine, lomustine, and streptozocin), platinum agents (i.e., alkylating-like agents) (e.g., carboplatin, cisplatin, oxaliplatin, BBR3464, and satraplatin), busulfan, dacarbazine, procarbazine, temozolomide, thioTEPA, treosulfan, and uramustine; antimetabolites such as folic acids (e.g., aminopterin, methotrexate, pemetrexed, and raltitrexed); purines such as cladribine, clofarabine, fludarabine, mercaptopurine, pentostatin, and thioguanine; pyrimidines such as capecitabine, cytarabine, fluorouracil, floxuridine, and gemcitabine; spindle poisonsmitotic inhibitors such as taxanes (e.g., docetaxel, paclitaxel, cabazitaxel) and vincas (e.g., vinblastine, vincristine, vindesine, and vinorelbine); cytotoxicantitumor antibiotics such anthracyclines (e.g., daunorubicin, doxorubicin, epirubicin, idarubicin, mitoxantrone, pixantrone, and valrubicin), compounds naturally produced by various species of streptomyces (e.g., actinomycin, bleomycin, mitomycin, plicamycin) and hydroxyurea; topoisomerase inhibitors such as camptotheca (e.g., camptothecin, topotecan and irinotecan) and podophyllums (e.g., etoposide, teniposide); monoclonal antibodies for cancer immunotherapy such as anti-receptor tyrosine kinases (e.g., cetuximab, panitumumab, trastuzumab), anti-CD20 (e.g., rituximab and tositumomab), and others for example alemtuzumab, bevacizumab, and gemtuzumab; photosensitizers such as aminolevulinic acid, methyl aminolevulinate, porfimer sodium, and verteporfin; tyrosine kinase inhibitors such as cediranib, dasatinib, erlotinib, gefitinib, imatinib, lapatinib, nilotinib, sorafenib, sunitinib, and vandetanib; serinethreonine kinase inhibitors, (e.g., inhibitors of AbI, c-Kit, insulin receptor family member(s), EGF receptor family member(s), Akt, mTOR (e.g., rapamycin or analogs thereof, direct inhibitors of mTORC1 and/or mTORC2), Raf kinase family, phosphatidyl inositol (PI) kinases such as PI3 kinase, PI kinase-like kinase family members, cyclin dependent kinase family members, aurora kinase family), growth factor receptor antagonists, and others such as retinoids (e.g., alitretinoin and tretinoin), altretamine, amsacrine, anagrelide, arsenic trioxide, asparaginase (e.g., pegaspargase), bexarotene, bortezomib, denileukin diftitox, estramustine, ixabepilone, masoprocol, mitotane, and testolactone, Hsp90 inhibitors, proteasome inhibitors, HDAC inhibitors, angiogenesis inhibitors, e.g., anti-vascular endothelial growth factor agents such as, bevacizumab or VEGF-Trap, matrix metalloproteinase inhibitors, pro-apoptotic agents (e.g., apoptosis inducers), anti-inflammatory agents, etc.

Because some drugs work better together than alone, two or more drugs are often given at the same time or sequentially. Often, two or more chemotherapy agents are used as combination chemotherapy. In some embodiments, the chemotherapy agents (including combination chemotherapy) can be used in combination with a compound or composition described herein. In some embodiments, a compound or composition described herein may be administered with another chemotherapeutic and another compound identified as being effective in the treatment or modulation of proliferation of cancer stem cells.

Targeted Therapy

In some embodiments, a compound or composition described herein is administered with a targeted therapy. Targeted therapy constitutes the use of agents specific for the deregulated proteins of cancer cells. Small molecule targeted therapy drugs are generally inhibitors of enzymatic domains on mutated, overexpressed, or otherwise critical proteins within the cancer cell. One example is tyrosine kinase inhibitors, e.g., a kinase inhibitor listed above, monoclonal antibody therapies, e.g., therapeutics comprising an antibody which specifically binds to a protein on the surface of the cancer cells, e.g., a monoclonal antibody therapy listed herein. Another example is PARP inhibitors, i.e., pharmacological inhibitors of the enzyme poly ADP ribose polymerase (PARP). Suitable PARP inhibitors may be iniparib, olaparib, rucaparib, veliparib, or CEP 9722. In some embodiments, the targeted therapy can be used in combination with a compound or composition described herein. Targeted therapy can also involve small peptides as “homing devices” which can bind to cell surface receptors or affected extracellular matrix surrounding the tumor. Radionuclides which are attached to these peptides (e.g., RGDs) eventually kill the cancer cell if the nuclide decays in the vicinity of the cell.

Immunotherapy

In some embodiments, a compound or composition described herein is administered with an immunotherapy. Cancer immunotherapy refers to a diverse set of therapeutic strategies designed to induce the subject's own immune system to fight the tumor. Contemporary methods for generating an immune response against tumors include intravesicular BCG immunotherapy for superficial bladder cancer, and use of interferons and other cytokines to induce an immune response in renal cell carcinoma and melanoma subjects.

Allogeneic hematopoietic stem cell transplantation can be considered a form of immunotherapy, since the donor's immune cells will often attack the tumor in a graft-versus-tumor effect. In some embodiments, the immunotherapy agents can be used in combination with a compound or composition described herein.

Hormonal Therapy

In some embodiments, a compound or composition described herein is administered with a hormonal therapy. The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial. Examples of hormonal therapies include tamoxifen (Nolvadex®, Istubal®, Valodex®), abarelix (Plenaxis®), flutamide (Eulexin®), bicalutamide (Casodex®), nilutamide (Nilandron®), an degarelix (Firmagon®). In some embodiments, the hormonal therapy agents can be used in combination with a compound or composition described herein.

Radiation Therapy

The formulations described herein can be used in combination with directed energy or particle, or radioisotope treatments, e.g., radiation therapies, e.g., radiation oncology, for the treatment of proliferative disease, e.g., cancer, e.g., cancer associated with cancer stem cells. The formulations may be administered to a subject simultaneously or sequentially along with the directed energy or particle, or radioisotope treatments. For example, the formulations may be administered before, during, or after the directed energy or particle, or radioisotope treatment, or a combination thereof. The directed energy or particle therapy may comprise total body irradiation, local body irradiation, or point irradiation. The directed energy or particle may originate from an accelerator, synchrotron, nuclear reaction, vacuum tube, laser, or from a radioisotope. The therapy may comprise external beam radiation therapy, teletherapy, brachytherapy, sealed source radiation therapy, systemic radioisotope therapy, or unsealed source radiotherapy. The therapy may comprise ingestion of, or placement in proximity to, a radioisotope, e.g., radioactive iodine, cobalt, cesium, potassium, bromine, fluorine, carbon. External beam radiation may comprise exposure to directed alpha particles, electrons (e.g., beta particles), protons, neutrons, positrons, or photons (e.g., radiowave, millimeter wave, microwave, infrared, visible, ultraviolet, X-ray, or gamma-ray photons). The radiation may be directed at any portion of the subject in need of treatment. The radiation may also be administered to cultured cells or cell samples, i.e., in vitro radiation therapy. In one embodiment, the cultured cells are cultured cancer stem cells.

Surgery

The compounds and compositions described herein can be used in combination with surgery, e.g., surgical exploration, intervention, biopsy, for the treatment of proliferative disease, e.g., cancer, e.g., cancer associated with cancer stem cells. The compounds and compositions may be administered to a subject simultaneously or sequentially along with the surgery. For example, the compounds or compositions may be administered before (pre-operative), during, or after (post-operative) the surgery, or a combination thereof. The surgery may be a biopsy during which one or more cells are collected for further analysis. The biopsy may be accomplished, for example, with a scalpel, a needle, a catheter, an endoscope, a spatula, or scissors. The biopsy may be an excisional biopsy, an incisional biopsy, a core biopsy, or a needle biopsy, e.g., a needle aspiration biopsy. The surgery may involve the removal of localized tissues suspected to be or identified as being cancerous. For example, the procedure may involve the removal of a cancerous lesion, lump, polyp, or mole. The procedure may involve the removal of larger amounts of tissue, such as breast, bone, skin, fat, or muscle. The procedure may involve removal of part of, or the entirety of, an organ or node, for example, lung, throat, tongue, bladder, cervix, ovary, testicle, lymph node, liver, pancreas, brain, eye, kidney, gallbladder, stomach, colon, rectum, or intestine. In one embodiment, the cancer is breast cancer, e.g., triple negative breast cancer, and the surgery is a mastectomy or lumpectomy.

Microbial Disorders

A compound or composition described herein can be used to treat a microbial growth or disorder. A “microbial disorder” is a disease or disorder characterized by growth of foreign cells on or within a subject, for example by a bacteria, virus, or fungus. The aqueous composition may target the cell wall or cell membrane of the microbes, or interfere with essential pathways thereby limiting the growth of the microbe. Exemplary microbial disorders include infection by coccidia, Staphylococcus aureus, Enterococcus faecalis and Enterococcus faecium, Streptococcus pneumoniae, E. coli, Salmonella, Klebsiella pneumoniae, Pseudomonas species and Enterobacter species.

### Microbial Combination Therapies

In some embodiments, a composition described herein is administered together with another antibiotic, e.g, a cephalosporin, a penicillin, a quinolone, a sulfonamide, or a tetracycline. Suitable antibiotics include abacavir, acyclovir, albendazole, amikacin, amoxicillin, ampicillin, azithromycin, aztreonam, benzilpenicillin, cefepime, ceftriaxone, cephalexin, chloramphenicol, chloroquine, cilastatin, clindamycin, co-trimoxazole, didanosine, dioxidine, doxycycline, famciclovir, fluconazole, fosfomycin, furazolidone, fusidic acid, ganciclovir, gentamicin, isoniazid, josamycin, kanamycin, ketoconazole, lamivudine, lincomycin, linezolid, mebendazole, meropenem, metronidazole, moxifloxacin, mupirocin, nystatin, nitrofuranton, nitroxoline, norfloxacin, ofloxacin, ornidazole, oseltamivir, polymixin B, polymyxin M, proguanil, ribavirin, rifampicin, rimantadine, roxithromycin, spectinomycin, sulfodimidin, teicoplanin, terbinafine, tetracycline, tinidazole, valaciclovir, valganciclovir, vancomycin, zanamivir, or zidovudine. One class of antibiotics, known as ionophores, includes lonomycin, ionomycin, laidlomycin, nigericin, grisorixin, dianemycin, lenoremycin, salinomycin, narasin, alborixin, septamycin, maduramicin, semduramicin, lasalocid, mutalomycin, isolasalocid A, lysocellin, tetronasin, and echeromycin.

### Methods of Use

A method for identifying compounds or compositions that inhibit cancer, e.g., a CSC-mediated tumor formation comprises contacting a test cell with a compound or composition and assaying for alterations in the growth and/or survival of the test cell. In some embodiments, a test cell is a cell that has undergone/is undergoing an epithelial to mesenchymal transition (e.g., a cell from the following cell lines: Ecad, GFP, Hs578T, MCF, Su159). The screening may be carried out in vitro or in vivo using an assay known to one of ordinary skill in the art to be suitable for contacting a test cell with a compound or composition and assaying for alterations in the growth and/or survival of the test cell. Some exemplary screening assays are described, but not limited to those set forth in WO 2009126310, which is incorporated by reference herein in its entirety.

### Methods of Administration and Dosage

The compounds and pharmaceutical compositions described herein may be administered orally, parenterally, by inhalation spray, topically, rectally, nasally, buccally, vaginally or via an implanted reservoir, preferably by oral administration or administration by injection. In some embodiments, the compound or pharmaceutical composition is administered to a subject parenterally. In some embodiments, the compound or pharmaceutical composition is dosed intravenously at a dose of 0.001 to 10 mg/kg, e.g., 0.005 to 5 mg/kg, e.g., 0.01 to 1 mg/kg, e.g., 0.1 to 1 mg/kg, e.g., 0.1, or 0.2, or 0.3, or 0.4, or 0.5, or 0.6, or 0.7, or 0.8, or 0.9, or 1.0 mg/kg. In some embodiments, the subject is administered the compound or pharmaceutical composition orally. In some embodiments, the compound or pharmaceutical composition is dosed orally at a dose of 0.01 to 100 mg/kg, e.g., 0.05 to 50 mg/kg, e.g., 0.1 to 10 mg/kg, e.g., 1 to 10 mg/kg, e.g., 2, or 2, or 3, or 4, or 5, or 6, or 7, or 8, or 9, or 10 mg/kg. The term parenteral as used herein includes subcutaneous, intracutaneous, intravenous, intramuscular, intraarticular, intraarterial, intrasynovial, intrasternal, intrathecal, intralesional and intracranial injection or infusion techniques. In some embodiments, the compound or pharmaceutical composition is configured for intravenous administration.

In some embodiments, the compound or pharmaceutical composition described herein is to be orally administered in any orally acceptable dosage form including, but not limited to, liqui-gel oral dosage form, syrups, emulsions and aqueous suspensions. Liqui-gels may include gelatins, plasticisers, and/or opacifiers, as needed to achieve a suitable consistency, and may be incorporated into a dosage form that is coated with an enteric coatings that is approved for such use, e.g., a shellac. Additional thickening agents, for example gums, e.g., xanthum gum, starches, e.g., corn starch, or glutens may be added to achieve a desired consistency of the compound or pharmaceutical composition when used as an oral dosage. If desired, certain sweetening and/or flavoring and/or coloring agents may be added.

In some embodiments, the method further comprises administering an additional cancer treatment e.g., radiation therapy, chemotherapy, or hormone therapy in combination with the compound or composition described herein. In some embodiments, the additional cancer treatment is administered simultaneously with the compound or pharmaceutical composition. In some embodiments, the additional cancer treatment is administered sequentially with the compound or pharmaceutical composition described herein. In some embodiments the additional cancer treatment is chemotherapy. In some embodiments, the chemotherapy is a taxane, e.g., docitaxel, paclitaxel, or cabazitaxel. In some embodiments, the chemotherapy is a platinum compound, e.g., cisplatin. In some embodiments, the chemotherapy is a PARP inhibitor, e.g., inaparib. In some embodiments, the chemotherapy is an anthracycline, e.g., doxorubicin.

Subject doses of the compound or pharmaceutical composition described herein typically range from about 0.1 μg to 10,000 mg, more typically from about 1 μg to 8000 mg, e.g., from about 10 μg to 100 mg once or more per day, week, month, or other time interval. Stated in terms of subject body weight, typical dosages in certain embodiments of the invention range from about 0.1 μg to 20 mg/kg/day, e.g., from about 1 to 10 mg/kg/day, e.g., from about 1 to 5 mg/kg/day. In some embodiments, the compound or pharmaceutical composition is dosed intravenously at a dose of 0.001 to 10 mg/kg, e.g., 0.005 to 5 mg/kg, e.g., 0.01 to 1 mg/kg, e.g., 0.1 to 1 mg/kg, e.g., 0.1, or 0.2, or 0.3, or 0.4, or 0.5, or 0.6, or 0.7, or 0.8, or 0.9, or 1.0 mg/kg. In some embodiments, the compound or pharmaceutical composition is dosed orally at a dose of 0.01 to 100 mg/kg, e.g., 0.05 to 50 mg/kg, e.g., 0.1 to 10 mg/kg, e.g., 1 to 10 mg/kg, e.g., 2, or 2, or 3, or 4, or 5, or 6, or 7, or 8, or 9, or 10 mg/kg. The absolute amount will depend upon a variety of factors including the concurrent treatment, the number of doses and the individual subject parameters including age, physical condition, size and weight. These are factors well known to those of ordinary skill in the art and can be addressed with no more than routine experimentation. It is often the case that a maximum dose be used, that is, the highest safe dose according to sound medical judgment. The dose used may be the maximal tolerated dose or a sub-therapeutic dose or any dose there between. Multiple doses of the molecules of the invention are also contemplated. When the molecules of the invention are administered in combination a sub-therapeutic dosage of either of the molecules, or a sub-therapeutic dosage of both, may be used in the treatment of a subject having, or at risk of developing, cancer. When the two classes of drugs are used together, the cancer medicament may be administered in a sub-therapeutic dose to produce a desirable therapeutic result. A sub-therapeutic dose is a dosage which is less than that dosage which would produce a therapeutic result in the subject if administered in the absence of the other agent. Thus, the sub-therapeutic dose of a cancer medicament is one which would not produce the desired therapeutic result in the subject in the absence of the administration of the molecules of the invention. Therapeutic doses of cancer medicaments are well known in the field of medicine for the treatment of cancer. These dosages have been extensively described in references such as Remington's Pharmaceutical Sciences, 18th ed., 1990; as well as many other medical references relied upon by the medical profession as guidance for the treatment of cancer.

Lower or higher doses than those recited above may be required. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Subjects may, however, require intermittent treatment on a long-term basis upon any recurrence of disease symptoms.

In some embodiments, the compound or pharmaceutical composition described herein is incorporated into a dosage form. In some embodiments, the dosage form is a parenteral dosage form, e.g., for administration to a subject intravenously. In some embodiments, the dosage form is composition in a sterile, sealed container (e.g., a bottle, a vial). In some embodiments, the dosage form may be an oral dosage form, e.g., for administration to a subject orally. In some embodiments, an oral dosage form additionally comprises flavors, or fragrances, or both, to modify the taste or odor of the oral dosage form.

### Methods of Evaluating Compounds

The efficacy of the compounds disclosed herein can be evaluated for their efficacy by contacting test cells and control cells with the compounds of interest. The test cells and control cells are then monitored for growth and/or survival. Compounds which result in different growth rates of the test cells compared to the control cells are selected for further testing and evaluation. For example, a panel of test cells may be contacted with different doses of the compound or a panel of test cells may be contacted with the compound for different durations of time. In some embodiments, the compounds are used to produce a response curve, wherein the test dose response curve indicates the level of inhibition of the test cells by the compound at a number of different doses. This analysis can be used to determine an EC50 value for the compound against the test cells and/or the control cells. In some cases, the EC50 value for the compound against the control cells is statistically significantly less than the EC50 value for the compound against the test cells. In other cases, the EC50 value for the compound against the control cells is statistically significantly greater than the EC50 value for the compound against the test cells.

In an embodiment, the compounds of the invention may be evaluated against cancer stem cells and/or mesenchymal cells using techniques disclosed in WO 2009126310, which is incorporated by reference in its entirety. In an embodiment, the compounds of the invention may be evaluated against cancer stem cells and/or mesenchymal cells using techniques disclosed in “Identification of Selective Inhibitors of Cancer Stem Cells by High-Throughput Screening” by Gupta et al., Cell, vol. 138, p. 645-659 (2009), which is incorporated by reference in its entirety.

Additionally, it is possible to compare the efficacy of the compound disclosed herein against control compounds, e.g., other cancer therapeutics (e.g., doxorubicin, paclitaxel, campothecin, actinomycin D, staurosporine), other antibiotics (e.g., penicillin, amoxicillin, tetracycline), or a combination thereof, using the methods described above.

### Subject Selection and Monitoring

In some embodiments, described herein, e.g., a subject suffering from or suspected of suffering from a disorder described herein or a sample taken from the subject, may be tested for the presence of a biomarker, e.g., one or more biomarkers associated with a cancer, e.g., a cancer stem cell, or a biomarker indicative of the presence of mesenchymal cells, prior to being administered compounds described herein. In some embodiments, the aqueous compositions are administered to a subject that has been identified with a predictive biomarker indicating the prevalence of CSCs, or tumor-initiating cells, or mesenchymal cells, or mesenchymal-like cells associated with cancer, or mesenchymal cancerous cells, or wherein the cancer is characterized as being enriched with CSCs or mesenchymal cells.

In order to identify or evaluate the biomarker, e.g., a cancer stem cell biomarker, or a biomarker indicative of the presence of mesenchymal cells, it may be necessary to obtain a clinical sample from the subject (e.g., a sample of the cancer). Typically, a clinical sample is a tumor biopsy or cells isolated there from. However, the invention is not so limited and any suitable clinical sample may be used, provided that the sample has a detectable cancer stem cell biomarker in a subject having a cancer stem cell. Exemplary clinical samples include saliva, hair folicles, gingival secretions, cerebrospinal fluid, gastrointestinal fluid, mucus, urogenital secretions, synovial fluid, blood, serum, plasma, urine, cystic fluid, lymph fluid, ascites, pleural effusion, interstitial fluid, intracellular fluid, ocular fluids, seminal fluid, mammary secretions, vitreal fluid, and nasal secretions.

In one embodiment, the clinical sample is screened for a genetic marker indicative of a disorder suitable for treatment with the compounds described herein, or for the presence of one or more genes correlated with a risk for developing a disorder suitable for treatment with the compounds described herein. For example, gene expression analysis (e.g., nucleic acid microarray, cDNA array, quantitative RT-PCR, RNAse protection assay) can be employed to identify specific genes or to locate markers indicative of genes related to the disorder. In some embodiments, one or more of the following genes, may be identified: ANAPC2, CCND1 (cyclin D1), CCNE1 (cyclin E1), CDC7, CDC34, CDK4, CDK6, CDKN1B (p27), CDKN1C (p57), CDKN3, CUL1, CUL2, CUL3, CUL4A, CUL5, E2F1, SKP2; S Phase and DNA Replication: ABL1 (C-ABL), MCM2, MCM3, MCM4 (CDC21), MCM5 (CDC46), MCM6 (Mis5), MCM7 (CDC47), PCNA, RP A3, SUMO1, UBE1; G2 Phase and G2M Transition: ANAPC2, ANAPC4, ANAPC5, ARHI, BCCIP, BIRC5, CCNA1 (cyclin A1), CCNB1 (cyclin B1), CCNG1 (cyclin G1), CCNH, CCNT1, CCNT2, CDC25A, CDC25C, CDC37, CDK5R1, CDK5R2, CDK5RAP1, CDK5RAP3, CDK2, CDK7, CDKN3, CKS1B, CKS2, DDX1 1, DNM2, GTF2H1, GTSE1, HERC5, KPNA2, MNAT1, PKMYT1, RGC32, SERTAD1; M Phase: CCNB2 (cyclin B2), CCNF, CDC2 (CDK1), CDC16, CDC20 (p55cdc), CDC25A, CDC25C, MRE1 IA, RAD50, RAD51; Cell Cycle Checkpoint and Cell Cycle Arrest: ATM, ATR, BRCA1, BRC A2, CCNA2 (cyclin A2), CCNE2 (cyclin E2), CCNG2 (cyclin G2), CDC2 (CDK1), CDC25A, CDC34, CDC45L, CDC6, CDK2, CDKN1A (p21), CDKN1B (p27), CDKN1C (p57), CDKN2A (p16), CDKN2B (p15), CDKN2C (p18), CDKN2D (p19), CDKN3, CHEK1 (CHK1), CHEK2 (CHK2 RAD53), CUL1, CUL2, CUL3, CUL4A, CUL5, GADD45A, HUS1, KNTC1, MAD2L1, MAD2L2, NBS1 (NIBRIN), RAD1, RAD17, RAD9A, RB1, RBBP8, TP53 (p53); Regulation of the Cell Cycle: ABL1 (C— ABL), ANAPC2, ANAPC4, ANAPC5, ARHI, ATM, ATR, BCC1P, BCL2, BRCA2, CCNA1 (cyclin A1), CCN A2 (cyclin A2), CCNB1 (cyclin B1), CCNB2 (cyclin B2), CCNC (cyclin C), CCND1 (cyclin D1), CCND2 (cyclin D2), CCND3 (cyclin D3), CCNE1 (cyclin E1), CCNE2 (cyclin E2), CCNF (cyclin F), CCNH (cyclin H), CCNT1, CCNT2, CDC 16, CDC2 (CDK1), CDC20 (p55cdc), CDC25A, CDC25C, CDC37, CDC45L, CDC6, CDK2, CDK4, CDK5R1, CDK5R2, CDK6, CDK7, CDK8, CDKN1A (p21), CDKN1B (p27), CKS1B, CUL5, DDX1 1, E2F1, E2F2, E2F3, E2F4, E2F5, E2F6, GADD45A, KNTC1, MKI67 (Ki67), PCNA, PKMYT1, RAD9A, RB1, SKP2, TFDP1 (DPI), TFDP2 (DP2); Negative Regulation of the Cell Cycle: ATM, BAX, BRCA1, CDC7, CDKN2B (p15), CDKN2D (p19), RBL1 (p107 RB), RBL2 (p130 RB2), TP53 (p53). Exemplary Cell Survival/Apoptotic Genes include those of the TNF Ligand Family: LTA (TNF-α), TNF (TNF-a), TNFSF5 (CD40 Ligand), TNFSF6 (FasL), TNFSF7 (CD27 Ligand), TNFSF8 (CD30 Ligand), TNFSF9 (4-IBB Ligand), TNFSF10 (TRAIL), TNFSF14 (HVEM-L), TNFSF18; the TNF Receptor Family: LTBR, TNFRSF1A (TNFR1), TNFRSF1B (TNFR2), TNFRSF5 (CD40), TNFRSF6 (Fas), TNFRSF6B, TNFRSF7 (CD27), TNFRSF9 (4-1BB), TNFRSF10A (DR4), TNFRSF10B (DR5), TNFRSF10C (DcR1), TNFRSF1OD (DcR2), TNFRSF1 IB, TNFRSF 12A, TNFRSF 14 (HVEM), TNFRSF 19, TNFRSF21, TNFRSF25; the Bcl-2 Family: BAD, BAG1, BAG3, BAG4, BAK1, BAX, BCL2, BCL2A1 (bfl-1), BCL2L1 (bcl-x), BCL2L2 (bcl-w), BCL2L10, BCL2L11 (bim-like protein), BCL2L12, BCL2L13, BCLAF1, BID, BIK, BNIP1, BNIP2, BNIP3 (nip3), BNIP3L, BOK (Mtd), HRK, MCL1; the Caspase Family: CASP1, CASP2, CASP3, CASP4, CASP5, CASP6, CASP7, CASP8, CASP9, CASP1O, CASP 14; the IAP Family: BIRC1 (NIAP), BIRC2 (IAP2), BIRC3 (IAP1), BIRC4 (XIAP), BIRC5 (Survivin), BIRC6 (Bruce), BIRC7, BIRC8; the TRAF Family: TRAF1, TRAF2, TRAF3 (CRAF1), TRAF4, TRAF5; the CARD Family: APAF1, BCL1O (HuE1O), BIRC2, BIRC3, CARD4 (NOD1), CARD6, CARD8, CARD9, CARD10, CARD1 1, CARD12, CARD14, CARD15, CASP1, CASP2, CASP4, CASP5, CASP9, CRADD, NOL3 (Nop30), PYCARD, RIPK2 (CARDIAC); the Death Domain Family: CRADD, DAPK1, DAPK2, FADD, RIPK1, TNFRSF10A, TNFRSF10B, TNFRSF1 IB, TNFRSF1A, TNFRSF21, TNFRSF25, TNFRSF6, TRADD; the CIDE Domain Family: CIDEA, CIDEB, DFFA, DFFB; the p53 and DNA Damage Response: ABL1, AKT1, APAF1, BAD, BAX, BCL2, BCL2L1, BID, CASP3, CASP6, CASP7, CASP9, GADD45A, TP53 (p53), TP53BP2, TP73, TP73L; and AKT1, BAG1, BAG3, BAG4, BCL2, BCL2A1, BCL2L1, BCL2L10, BCL2L2, BFAR, BIRC1, BIRC2, BIRC3, BIRC4, BIRC5, BIRC6, BIRC7, BIRC8, BNIP1, BNIP2, BNIP3, BRAF, CASP2, CFLAR, GDNF, IGF1R, MCL1, TNF (TNF-a), TNFRSF6, TNFRSF6B, TNFRSF7, TNFSF 18, TNFSF5.

In one embodiment, a stem cell biomarker, or a biomarker indicative of the presence of mesenchymal cells, is selected from E-cadherin, TWIST expression, and a CD44CD24 cell surface marker profile. The stem cell biomarker, or the biomarker indicative of the presence of mesenchymal cells, may be identified in a sample of a cancer obtained from the subject. In one embodiment, the E-cadherin and/or TWIST expression in the cancer is determining by measuring a level of E-cadherin and/or TWIST protein and/or RNA expression in the cancer, and optionally comparing the level to a reference standard. In one embodiment, the reference standard is the level of E-cadherin and/or TWIST protein and/or RNA expression in a cancer stem cell. In one embodiment, the reference standard is the level of E-cadherin and/or TWIST protein and/or RNA expression in a cancer cell that is not a cancer stem cell.

In one embodiment, the stem cell biomarker, or a biomarker indicative of the presence of mesenchymal cells, is selected from CD20, CD24, CD34, CD38, CD44, CD45, CD105, CD133, CD166, EpCAM, ESA, SCA1, Pecam, and Strol.

In some cases it may be desirable to evaluate a cancer stem cell biomarker, or a biomarker indicative of the presence of mesenchymal cells, in a subject having, or suspect of having, cancer, and to select a treatment for the subject based on the results of the biomarker evaluation. For example, if the cancer stem cell biomarker, or the biomarker indicative of the presence of mesenchymal cells, is detected, the subject may be treated with an effective amount of a compound or composition disclosed herein. In some embodiments, if the cancer stem cell biomarker, or the biomarker indicative of the presence of mesenchymal cells, is detected, the subject may be treated with an effective amount of a pharmaceutical composition comprising abamectin, etoposide or nigericin, or a derivative of any of the foregoing, optionally in combination with paclitaxel or a derivative thereof (e.g., water-soluble or targeted derivatives or structurally related compounds, e.g., analogs such as docetaxel (see, e.g., WO/2003/045932 and US2008033189). The cancer stem cell biomarker, or the biomarker indicative of the presence of mesenchymal cells, of the foregoing methods may be evaluated using methods disclosed herein or any suitable methods known in the art. Exemplary cancer stem cell biomarkers, or biomarkers indicative of the presence of mesenchymal cells, include E-cadherin expression, TWIST expression, and a CD44⁺ CD24 marker profile. Other biomarkers may indicate activity in a pathway selected from TGF-β, Wnt, BMP, Notch, HGF-Met, EGF, IGF, PDGF, FGF, P38-mapk, Ras, PB Kinase-Akt, Src, and NF-kB. Other exemplary cancer stem cell biomarkers, or biomarkers indicative of the presence of mesenchymal cells, are disclosed herein and will be apparent to one of ordinary skill in the art.

In one embodiment, the clinical sample may be screened for protein levels, for example the level of protein encoded by a cell cyclegrowth and/or survival gene, e.g., a gene listed above. Protein levels can be assessed by an appropriate method known to one of ordinary skill in the art, such as western analysis. Other methods known to one of ordinary skill in the art could be employed to analyze proteins levels, for example immunohistochemistry, immunocytochemistry, ELISA, radioimmunoassays, and proteomics methods, such as mass spectroscopy or antibody arrays.

After having been identified with a biomarker, a subject receiving a compound or composition described herein can be monitored, for example, for improvement in the condition and/or adverse effects, or for the expression of biomarkers indicative of the disorder. Improvement of a subject's condition can be evaluated, for example, by monitoring the growth, absence of growth, or regression of the cancer (e.g., a tumor). In some embodiments, the subject is evaluated using a radiological assay or evaluation of hemolytic parameters. In other embodiments the subject may be evaluated using gene or protein assays described herein. The subject may also be evaluated using conventional screening methods, such as physical exam, mammography, biopsy, colonoscopy, etc.

### Kits

The invention additionally includes kits comprising compositions described herein. In some embodiments, the kit additionally comprises a diluent for the purpose of diluting the aqueous composition as it is received in the kit. In some embodiments, the diluent is water. In some embodiments, the diluent is a pharmaceutically-acceptable vehicle, e.g., a vehicle disclosed herein. In some embodiments, the diluent comprises water. In some embodiments, the kit comprises instructions for diluting the aqueous composition with the diluent included in the kit.

In some embodiments, the kit comprises an additionally therapeutic agent, e.g., a chemotherapeutic, e.g., a chemotherapeutic agent described herein. In some embodiments, the kit additionally comprises instructions for administering the aqueous composition along with the additional therapeutic agent.

The aqueous compositions described herein may be administered to a subject as a compound or composition or dosage form. In some cases the aqueous compositions or dosage forms may be part of a kit, along with instructions for administering the aqueous composition. The kit may additionally comprise a diluent (e.g., water, saline, or a vehicle described herein) and instructions for administering the diluents along with the aqueous composition as intended. The aqueous compositions may be administered along with additional therapeutic agents, if present, in amounts effective for achieving a modulation of disease or disease symptoms, including those described herein. The additional therapeutic agents may be administered simultaneous with the aqueous compositions described herein, or they may be administered sequentially with the aqueous compositions described herein.

### EXAMPLES

The following Examples relate to Schemes 1-7 (below).

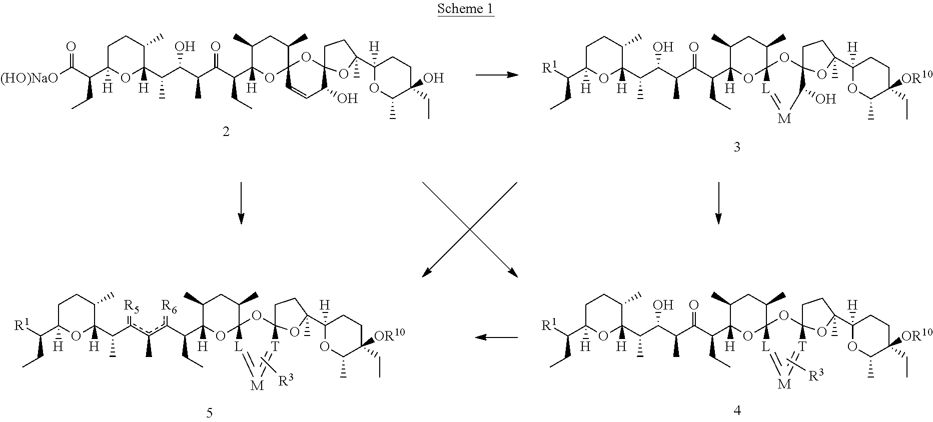

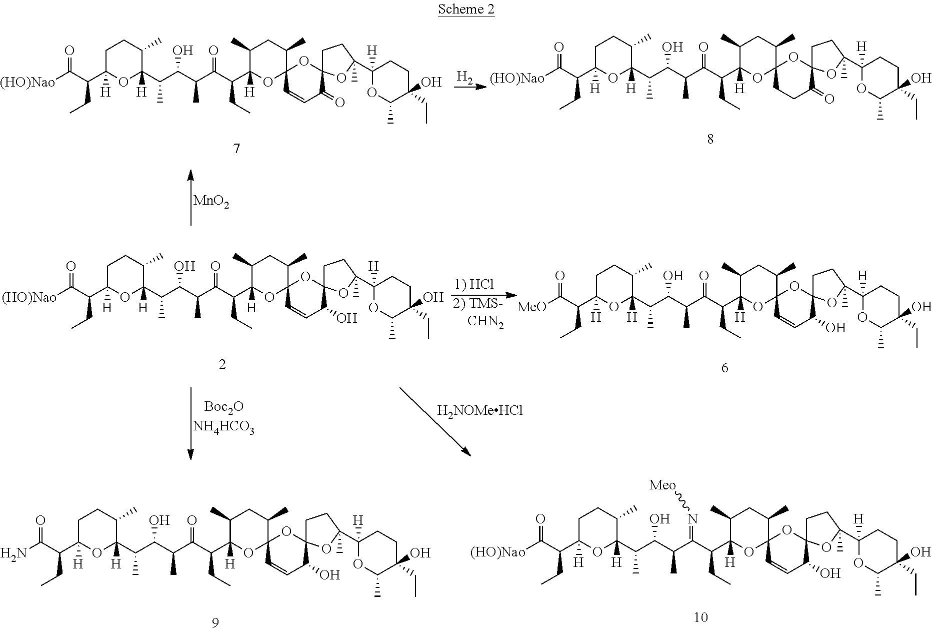

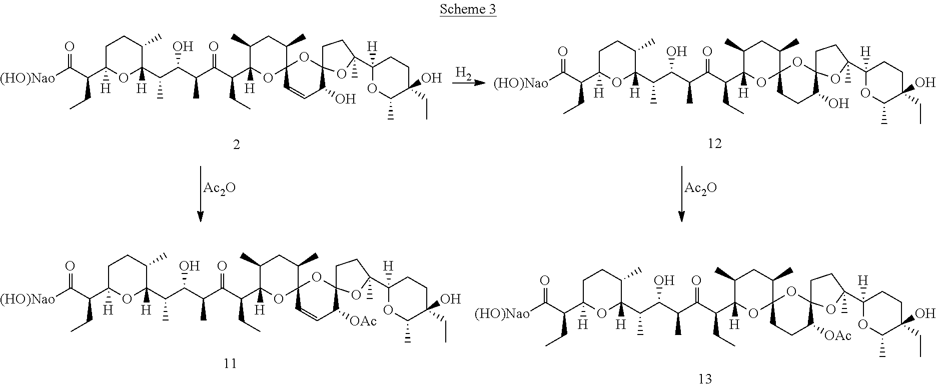

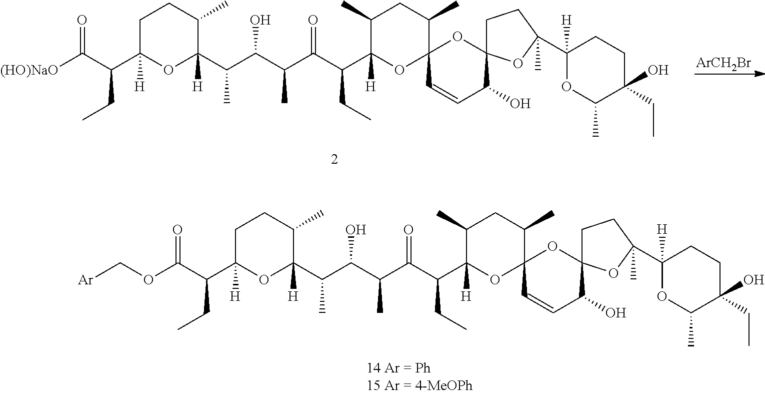

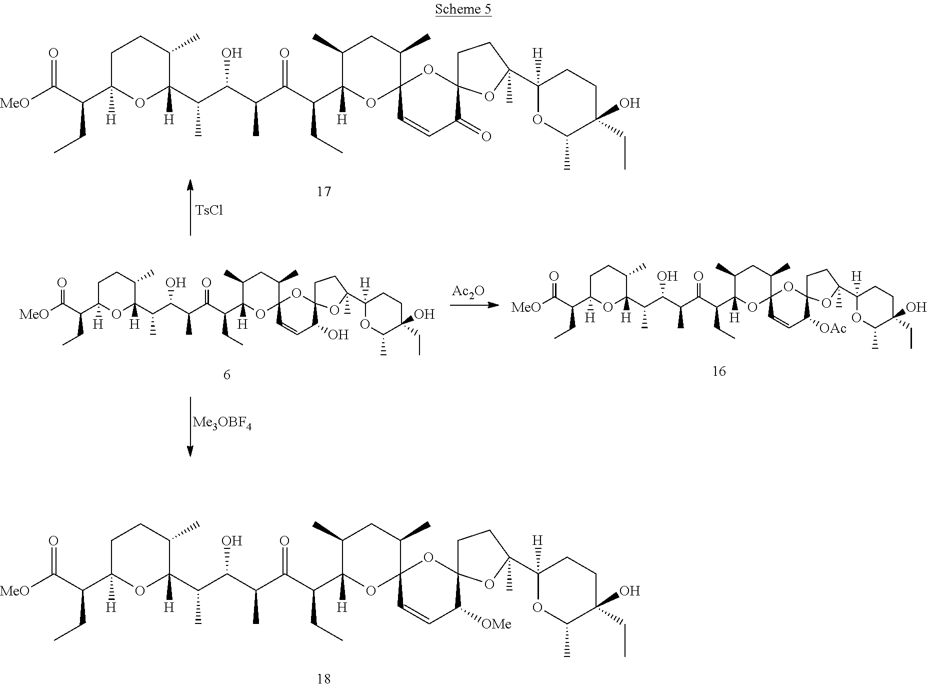

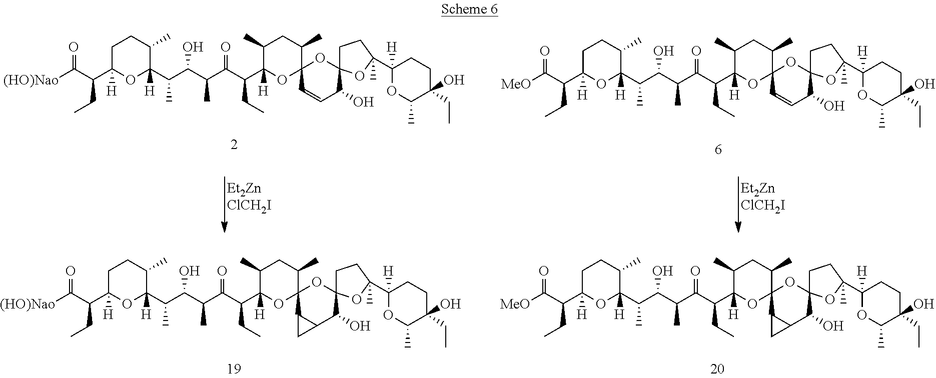

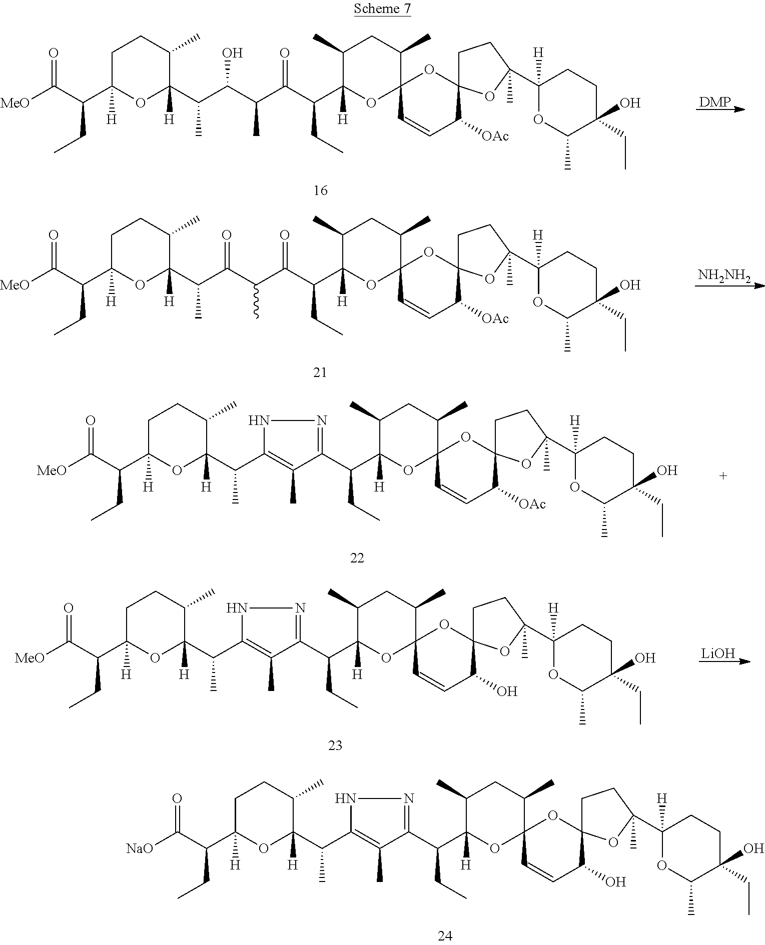

The compounds in Schemes 1-7 are numbered for convenience, and need not relate to any specific compound in Table 1 (above). Furthermore, the synthetic methods described below, while enabling, are merely exemplary of the methods which can be used to obtain the compounds described herein. One of skill in the art could readily modify the Examples to achieve the claimed compounds. Reagents disclosed below (e.g., EtOAc, HCl, MeOH, etc.) are available from multiple suppliers, for example, Sigma-Aldrich (Milwaukee, Wis.).

### Example 1

### Production of Salinomycin Methyl Ester (6)

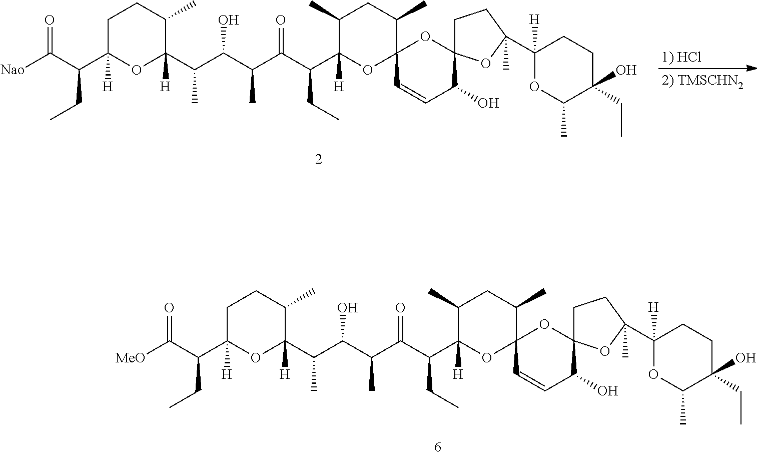

Salinomycin sodium salt (2, 10 g, Zhejiang Shenghua Baike Pharmaceutical, China) was dissolved in EtOAc (250 mL) and washed with HCl 0.1 N (250 mL). The organic phase was washed with brine (50 mL), dried over magnesium sulfate, filtered and concentrated under vacuum. The residue was dissolved in a CHCl₃—MeOH mixture (1:1, 100 mL) and the resulting solution was cooled to 0° C. TMSCHN₂ (2.0 M in Et₂O, 6.30 mL) was added over 15 min, the resulting solution was stirred for 1 h at rt and concentrated under vacuum. The crude product was purified by chromatography (silica gel saturated with triethylamine, EtOAc-hexane) on a RediSep® Gold column (Teledyne Isco, Lincoln, Nebr.) using a Teledyne Isco purification system to yield 8.32 g (84% yield) of product 6 as a white solid. MS (ESI+): 787.70 (M+Na)⁺.

In an alternate synthetic method, diazomethane was generated by decomposition of n-nitroso methyl guanidine. An excess of the yellow diazomethane solution was added via pipet to the solution of salinomycin sodium salt (2, 0.2 g, 1.0 eq.) dissolved in anhydrous diethyl ether (5 ml). Diazomethane solution was added to the reaction until the yellow color persisted. Then the reaction was stirred for 1 h at ambient temperature. The reaction was quenched with 1 drop of acetic acid and was diluted with ethyl acetate. It was washed with saturated sodium bicarbonate, dried over magnesium sulfate and concentrated in vacuo. The crude product was purified by flash chromatography using a 25S Biotage silica column (Biotage AB, Uppsala, Sweden) using ethyl acetatehexane gradient. The pure product was isolated in 58% yield. MS 764.5 (M+Na); calc. exact mass 787.5.

### Example 2

### Production of 20-acetoxy-salinomycin methyl ester (6a)

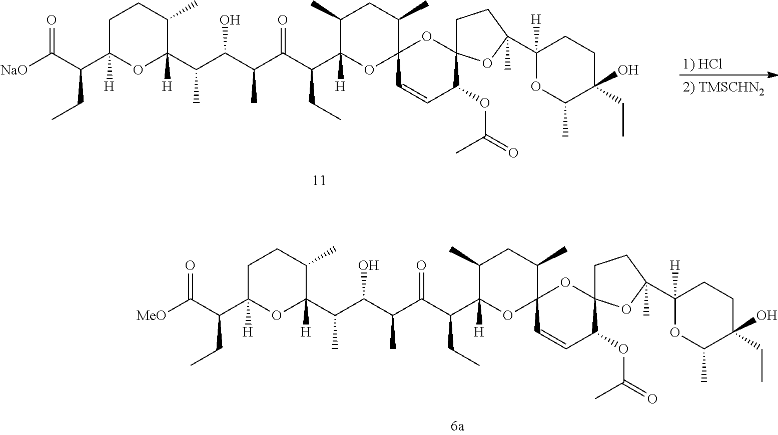

Salinomycin methyl ester 6a was prepared analogously to methyl ester 6 (Example 1), starting with acetate 11. The product 6a was isolated in 60% yield after chromatography. MS 829.5 (M+Na); calc. exact mass 806.5.

### Example 3

### Production of 20-oxo-salinomycin (7)

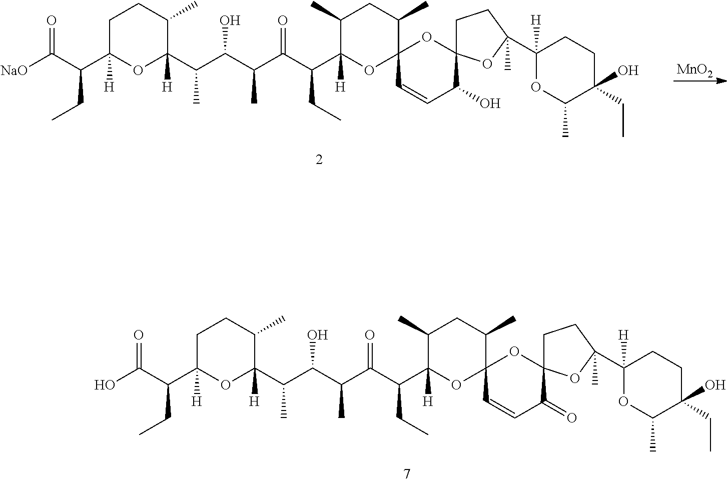

MnO₂ (163 mg, 15.0 eq.) was added to a solution of salinomycin sodium salt (2, 100 mg, 1.0 eq.) in CH₂Cl₂ (1.5 mL), the mixture was stirred for 24 h at rt, filtered on celite and concentrated. The residue was dissolved in CH₂Cl₂ (1.5 mL) and MnO₂ (163 mg, 15.0 eq.) was added. The resulting mixture was stirred for 48 h at rt, filtered on celite and concentrated. The residue was dissolved in EtOAc (5 mL), washed with HCl 0.1 N (2 mL), dried over magnesium sulfate, filtered and concentrated under vacuum. The crude product was purified by chromatography (silica gel, CH₂Cl₂—MeOH) on a RediSep® Gold column using Teledyne Isco purification system to yield 54 mg (54% yield) of product 7 as a white solid. MS (ESI−): 747.54 (M−H)⁻.

### Example 4

### Production of 18,19-dihydro-20-oxo-salinomycin (8)

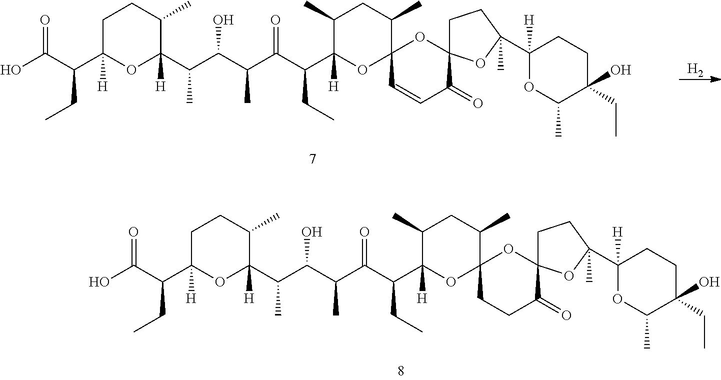 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=CC(=O)[C@]3(CC[C@@](C)([C@H]4CC[C@](O)(CC)[C@H](C)O4)O3)O2)[C@H](C)C[C@@H]1C ; "H2" ; CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(CCC(=O)[C@]3(CC[C@@](C)([C@H]4CC[C@](O)(CC)[C@H](C)O4)O3)O2)[C@H](C)C[C@@H]1C]

20-Oxo-salinomycin (7, 50 mg, 1.0 eq.) (Example 3) was dissolved in EtOAc (1 mL) and Pd/C (10%, 50% wet, 50 mg) was added. H₂ was bubbled in the mixture for 10 min before the reaction was stirred for 16 h. N₂ was bubbled for 10 min, the mixture was filtered on Celite® and the filtrate was concentrated under vacuum to yield 48 mg (96%) of product 8 as a white solid. MS (ESI+): 773.53 (M+Na)⁺.

### Example 5

### Production of Salinomycin Carboxamide (9)

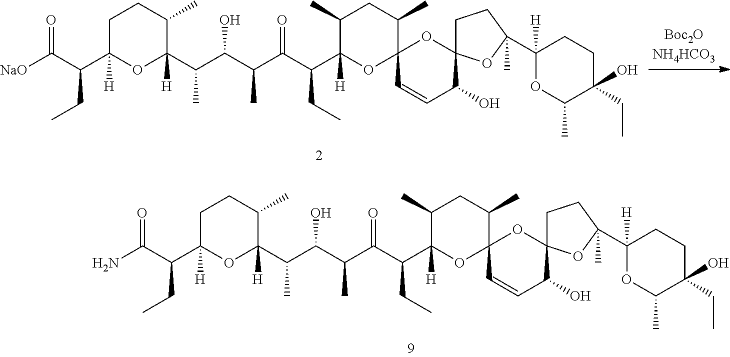

A mixture of salinomycin sodium salt (2, 300 mg, 1.0 eq), di-t-butyl-dicarbonate (439 mg, 5.2 eq.), NH₄HCO₃ (398 mg, 5.0 eq.) and MeCN (1.3 ml) were mixed and pyridine (0.02 mL, 0.5 eq.) was added. The mixture was stirred for 60 h at rt. Di-t-butyl-dicarbonate (439 mg, 5.2 eq.), NH₄HCO₃ (398 mg, 5.0 eq.) and pyridine (0.03 mL, 0.75 eq.) were added and the mixture was heated at 40° C. for 2 h. Di-t-butyl-dicarbonate (439 mg, 5.2 eq.), NH₄HCO₃ (398 mg, 5.0 eq.) and MeCN (1 mL) were added and the mixture was heated at 40° C. for 16 h. EtOAc (100 mL) was added and the afforded mixture was washed with water (50 mL) then with brine (25 mL). The organic phase was dried over sodium sulfate, filtered and concentrated under vacuum. The crude product was purified by chromatography (silica gel, ethyl acetate-hexane-acetone) on a RediSep® Gold column using Teledyne Isco purification system to yield 125 mg (43%) of product. Trituration with acetone afforded 92 mg of 9 as a white powder (32%). MS (ESI+): 772.65 (M+Na)⁺.

### Example 6

### Production of 11-methyloxime-salinomycin sodium salt (10)

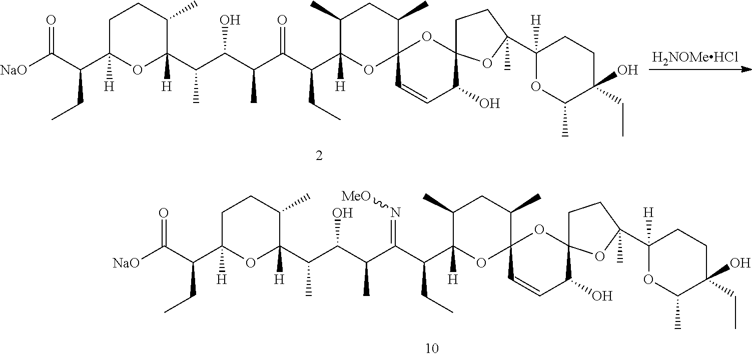

A mixture of salinomycin sodium salt (2, 300 mg, 1.0 eq), O-methylhydroxylamine (470 mg, 14.5 eq.), pyridine (0.86 mL, 27 eq.) and MeOH (3.9 ml) was stirred for 84 h at rt. EtOAc (50 mL) was added and the afforded mixture was washed with aq. HCl 1 N (25 mL), water (25 mL), sat. aq. NaHCO₃ (25 mL) and brine (25 mL). The organic phase was dried over sodium sulfate, filtered and concentrated under vacuum. The crude product was purified by chromatography (silica gel, ethyl acetate-hexane-acetone) on a RediSep® Gold column using Teledyne Isco purification system to yield 78 mg (26%) of 10 as an oil. MS (ESI+): 802.60 (M+H)⁺.

### Example 7

### Production of 20-acetoxy-salinomycin sodium salt (11)

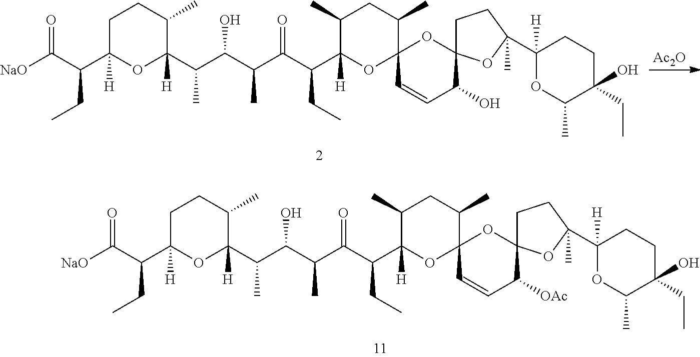

Ac₂O (2.44 mL, 8.0 eq.) was added slowly to a solution of salinomycin sodium salt (2, 2.50 g, 1.0 eq) and DMAP (20 mg) in pyridine (12 mL) at 0° C. The reaction mixture was stirred for 16 h at rt, then cooled to 0° C. before addition of water (20 mL). The mixture was stirred for 30 min at rt before addition of EtOAc (50 mL) and HCl 6 N (24 mL). Phases were separated and the organic phase was washed with HCl 0.1 N (20 mL), brine (20 mL), aq. sat. NaHCO₃ (2×20 mL) then with brine (20 mL). The organic phase was dried over sodium sulfate, filtered and concentrated under vacuum. The crude product was crystallized from acetone-water mixture to yield 1.95 g (74%) of 11 as a white solid. MS (ESI+): 815.70 (M+H)⁺.

In an alternate synthetic method, a solution of salinomycin sodium salt (2, 0.15 g, 1.0 eq.) in dry pyridine (1 ml) was added to acetic anhydride (0.075 ml) under ice cooling. The reaction was stirred at ambient temperature under nitrogen for 18 h. Afterwards, the reaction was poured into ice water and acidified to pH 3 with 0.05 N HCl. The aqueous layer was back-extracted with ethyl acetate (twice). The organic layer was dried over magnesium sulfate and concentrated in vacuo. The crude product was purified by flash chromatography using a 25M Biotage silica column using ethyl acetatehexane gradient. The pure product 11 was isolated in 41% yield. MS 814.5 (M+Na); calc. exact mass 792.5.

### Example 8

### Production of 18,19-dihydro-salinomycin sodium salt (12)

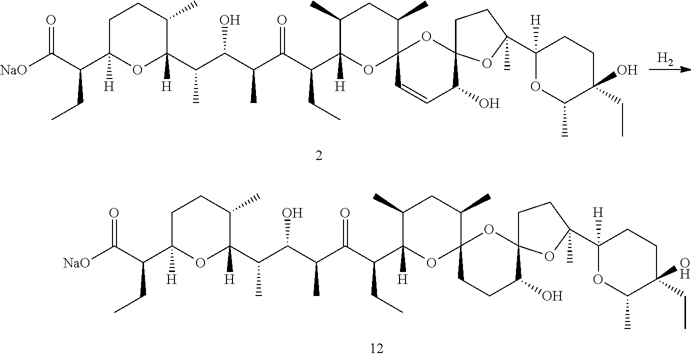

Salinomycin sodium salt (2, 325 mg, 1.0 eq.) was dissolved in THF (5 mL) and PdC (10%, 50 mg) was added. H₂ was bubbled in the mixture for 10 min before the reaction was stirred for 16 h. The mixture was filtered on a 2 micron filter and the filtrate was concentrated under vacuum to yield 313 mg (96%) of 12 as a white solid. MS (ESI+): 775.61 (M+H)⁺.

In an alternate synthetic method, a solution of salinomycin sodium salt (2, 0.2 g, 0.26 mmol, 1.0 eq.) in ethyl acetate (10 ml) was added platinum oxide (0.1 eq). The reaction was evacuated and backfilled with hydrogen gas (3 times). The reaction was stirred at ambient temperature under a hydrogen balloon for 18 h. The reaction mixture was then filtered through celite and concentrated in vacuo. The crude product was purified by flash chromatography using a 25S Biotage silica column using ethyl acetatehexane gradient. The pure product (white foam) was isolated in 30% yield. MS 777.2 (M+Na).

### Example 9

### Production of 18,19-dihydro-20-acetoxy-salinomycin sodium salt (13)

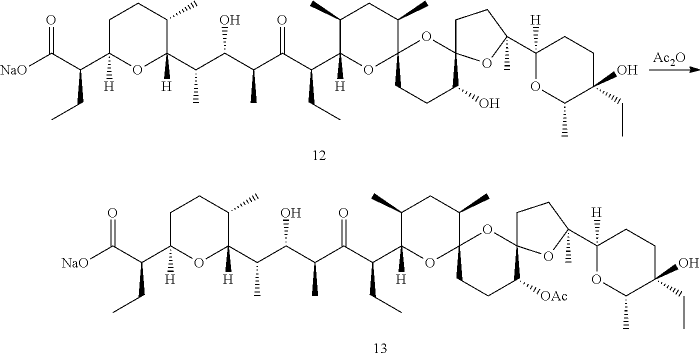

Ac₂O (0.26 mL, 8.0 eq.) was added slowly to a solution of (12, 268 mg, 1.0 eq) (Example 8) and DMAP (1 mg) in pyridine (1.3 mL) at 0° C. The reaction mixture was stirred for 16 h at rt, then Ac₂O (0.13 mL, 4.0 eq.) was added. The reaction mixture was stirred for 5 h at rt and EtOAc (5 mL) was added. The mixture was stirred for 30 min at rt before addition of hexane (5 mL). Phases were separated and the organic phase was washed with HCl 1 N (15 mL), HCl 0.1 N (10 mL), brine (10 mL), aq. sat. NaHCO₃ (10 mL) and brine (10 mL). The organic phase was dried over sodium sulfate, filtered and concentrated under vacuum. The crude product was crystallized from acetone to yield 140 mg (49%) of 13 as a white solid. MS (ESI+): 817.63 (M+H)⁺.

### Example 10

### Production of Salinomycin Benzyl Ester (14)

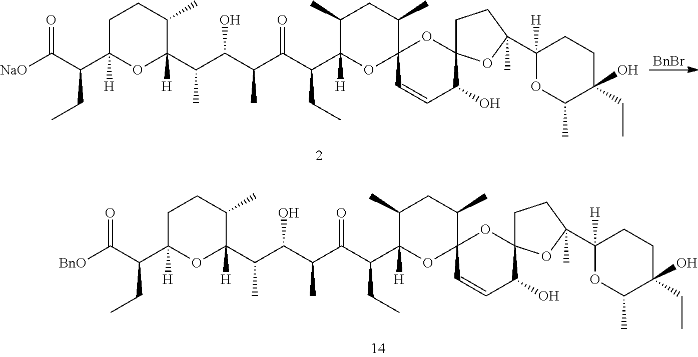

A mixture of salinomycin sodium salt (2, 1.00 g, 1.0 eq.), BnBr (0.77 mL, 5.0 eq.), NaHCO₃ (1.08 g, 10.0 eq.) and DMF (5 ml) was stirred protected from light for 80 h at rt. EtOAc (75 mL) was added and the afforded mixture was washed with water (5×50 mL) then with brine (25 mL). The organic phase was dried over magnesium sulfate, filtered and concentrated under vacuum. The crude product was purified by chromatography (silica gel saturated with triethylamine, ethyl acetate-hexane) on a RediSep® Gold column using Teledyne Isco purification system to yield 0.83 g (76% yield) of 14 as a white solid. MS (ESI+): 863.7 (M+Na)⁺.

In an alternate synthetic method, a solution of salinomycin sodium salt (2, 0.15 g, 0.19 mmol, 1.0 eq.) in DMF (2 ml) was added to sodium bicarbonate (32 mg, 2.0 eq.) and benzyl bromide (0.16 g, 5.0 eq.) and the reaction was stirred at ambient temperature under nitrogen for 18 h. Afterwards, the reaction was diluted with water and extracted with ethyl acetate (twice). The organic layer was dried over magnesium sulfate and concentrated in vacuo. The crude product was purified by flash chromatography using a 25M Biotage silica column using ethyl acetatehexane gradient. The pure product was isolated in 40% yield. MS 862.5 (M+Na); calc. exact mass 840.5.

### Example 11

### Production of Salinomycin 4-Methoxybenzyl Ester (15)

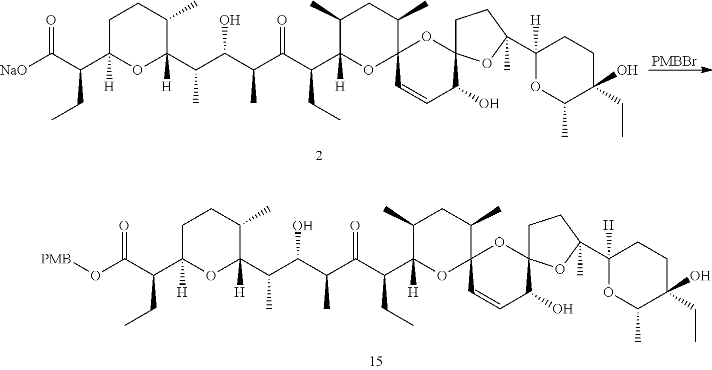

A mixture of salinomycin sodium salt (2, 2.00 g, 1.0 eq.), PMBBr (1.56 g, 3.0 eq.), NaHCO₃ (0.84 g, 4.0 eq.), DMF (5.2 ml) were stirred protected from light for 80 h at rt. EtOAc-hexane mixture (3:1, 20 mL) was added and the afforded mixture was washed with water (4×25 mL) then with brine (25 mL). The organic phase was dried over sodium sulfate, filtered and concentrated under vacuum. The crude product was purified by chromatography (silica gel saturated with triethylamine, ethyl acetate-hexane) on a RediSep® Gold column using Teledyne Isco purification system to yield 1.88 g (84% yield) of 15 as a white solid. MS (ESI+): 888.69 (M+NH₄)⁺.

### Example 12

### Production of 20-acetoxy-salinomycin methyl ester (16)

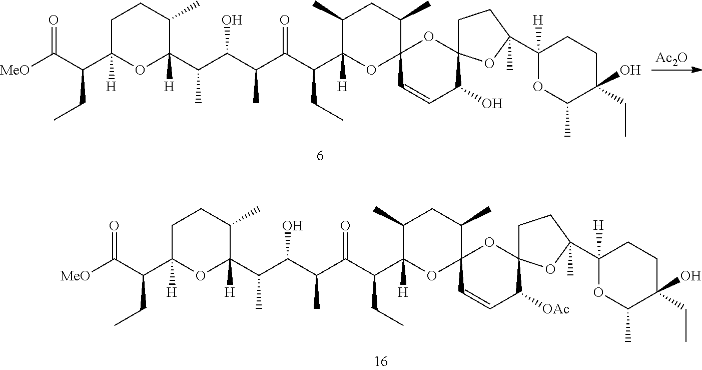 [SMILES: "Ac2O" ; CC[C@@H](C(=O)[C@@H](C)[C@@H](O)[C@H](C)[C@@H]1O[C@@H]([C@@H](CC)C(=O)OC)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C ; CC[C@@H](C(=O)[C@@H](C)[C@@H](O)[C@H](C)[C@@H]1O[C@@H]([C@@H](CC)C(=O)OC)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C]

Ac₂O (5.90 mL, 12.0 eq.) was added slowly to a solution of (6, 4.00 g, 1.0 eq) (Example 1) and DMAP (20 mg) in pyridine (20 mL) at 0° C. The reaction mixture was stirred for 1 h at rt, diluted with EtOAc (250 mL), washed with HCl 1 N (250 mL), HCl 0.1 N (100 mL), sat. aq. NaHCO₃ (100 mL) then with brine (100 mL). The organic phase was dried over magnesium sulfate, filtered and concentrated under vacuum. The crude product was purified by chromatography (silica gel saturated with triethylamine, ethyl acetate-hexane) on a RediSep® Gold column using Teledyne Isco purification system to yield 3.81 g (90% yield) of 16 as a white powder. MS (ESI+): 829.69 (M+Na)⁺.

### Example 13

### Production of 20-p-toluenesulfonyloxy-salinomycin methyl ester(17)

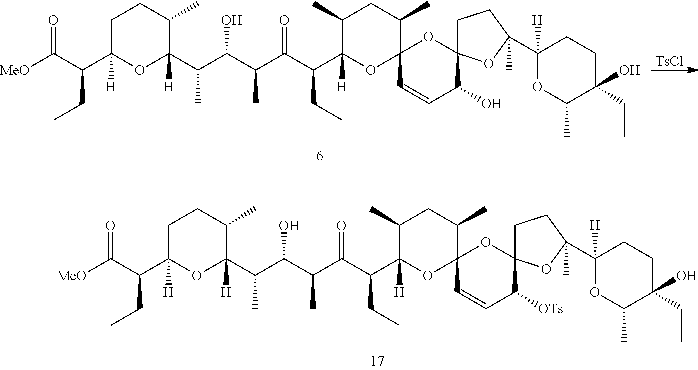 [SMILES: CC[C@@H](C(=O)[C@@H](C)[C@@H](O)[C@H](C)[C@@H]1O[C@@H]([C@@H](CC)C(=O)OC)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C ; "TsCl" ; CC[C@@H](C(=O)[C@@H](C)[C@@H](O)[C@H](C)[C@@H]1O[C@@H]([C@@H](CC)C(=O)OC)CC[C@@H]1C)[C@H]1O[C@]2(C=C[C@@H](OS(=O)(=O)c3ccc(C)cc3)[C@]3(CC[C@@](C)([C@H]4CC[C@](O)(CC)[C@H](C)O4)O3)O2)[C@H](C)C[C@@H]1C]

Salinomycin methyl ester (6) (267 mg, 1.0 eq) (Example 1) and DMAP (2.1 mg) were mixed in pyridine (1.3 mL) at 0° C. and p-toluenesulfonyl chloride (533 mg, 8.0 eq.) was added in one portion. The reaction mixture was stirred for 4 h at rt. The mixture was then cooled to 0° C. before addition of water (5 mL) and EtOAc (5 mL). The mixture was stirred for 30 minutes at rt and hexane (5 mL) was added. Phases were separated and the organic phase was washed with HCl 1 N (15 mL), HCl 0.1 N (10 mL), brine (10 mL), aq. sat. NaHCO₃ (10 mL) and brine (10 mL). The organic phase was dried over sodium sulfate, filtered and concentrated under vacuum to yield 316 mg (98% yield) of 17 as a white solid. MS (ESI+): 936.66 (M+NH₄)⁺.

### Example 14

### Production of 20-methoxy-salinomycin methyl ester (18)

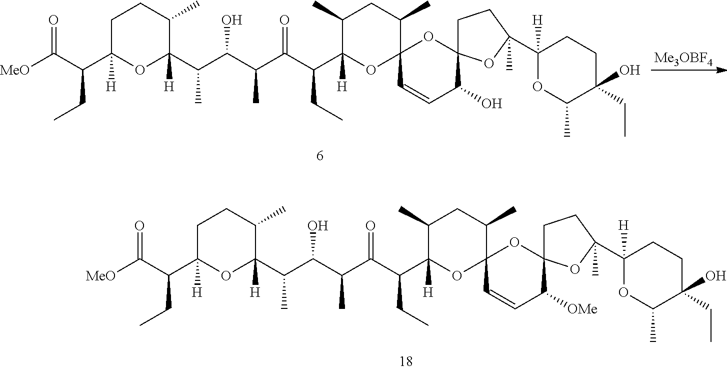 [SMILES: "Me3OBF4" ; CC[C@@H](C(=O)[C@@H](C)[C@@H](O)[C@H](C)[C@@H]1O[C@@H]([C@@H](CC)C(=O)OC)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C ; CC[C@@H](C(=O)[C@@H](C)[C@@H](O)[C@H](C)[C@@H]1O[C@@H]([C@@H](CC)C(=O)OC)CC[C@@H]1C)[C@H]1O[C@]2(C=C[C@@H](OC)[C@]3(CC[C@@](C)([C@H]4CC[C@](O)(CC)[C@H](C)O4)O3)O2)[C@H](C)C[C@@H]1C]

Salinomycin methyl ester (6) (104 mg, 1.0 eq.) (Example 1) and 1,8-bis(dimethylamino)naphthalene (Proton Sponge®, Sigma-Aldrich, 38 mg, 1.3 eq.) were dissolved in CH₂Cl₂ (1.1 mL) at rt, before addition of trimethyloxonium tetrafluoroborate (24 mg, 1 eq.). The reaction mixture was stirred for 16 h at rt and Proton Sponge® (190 mg, 6.5 eq.), 4 Å molecular sieves (500 mg) and trimethyloxonium tetrafluoroborate (120 mg, 6.0 eq.) were added. The mixture was stirred for 16 h and was concentrated under vacuum. The mixture was purified by chromatography (silica gel saturated with triethylamine, ethyl acetate-hexane) on a RediSep®® Gold column using Teledyne Isco purification system to yield 15 mg (14% yield) of 18. MS (ESI+): 796.62 (M+NH₄)⁺.

### Example 15

### Production of 18,19-methylene-salinomycin (19)

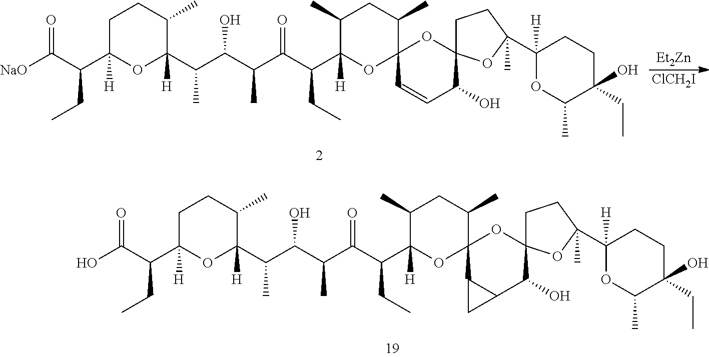

Diethyl zinc (1.1 M in PhMe, 0.70 mL, 6.0 eq.) was added to a solution of ClCH₂I (112 μL, 12.0 eq.) in CH₂Cl₂ (2 mL) at 0° C. The resulting mixture was stirred for 10 min at 0° C. before a solution of salinomycin sodium salt (2, 100 mg, 1.0 eq.) in CH₂Cl₂ (1 mL) was added. The mixture was stirred for 96 h at rt. Aq. sat. NH₄Cl (2 mL) was added and the reaction mixture was extracted with CH₂Cl₂. The organic phase was concentrated, the residue was dissolved in EtOAc (5 mL) and HCl 0.1 N (5 mL) was added. Layers were separated, the organic phase was washed with brine, dried over magnesium sulfate, filtered and concentrated under vacuum to yield 88 mg (86% yield) of 19. MS (ESI+): 787.63 (M+Na)⁺.

### Example 16

### Production of 18,19-methylene-salinomycin methyl ester (20)

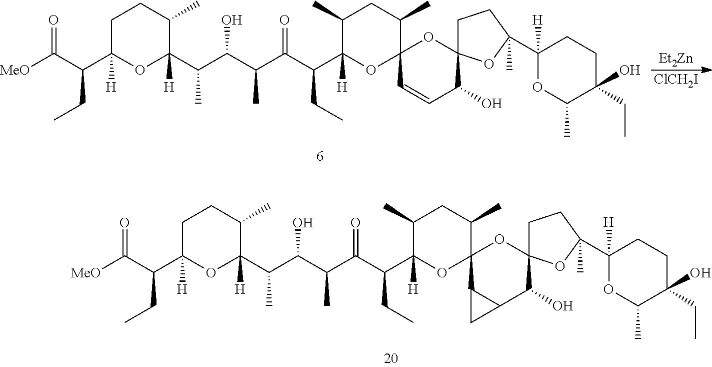

Diethyl zinc (1.1 M in PhMe, 0.30 mL, 5.0 eq.) was added to a solution of C CH₂I (47 μL, 10.0 eq.) in CH₂Cl₂ (1 mL) at 0° C. The resulting mixture was stirred for 10 min at 0° C. before a solution of salinomycin methyl ester (6, 50 mg, 1.0 eq.) (Example 1) in CH₂Cl₂ (1 mL) was added. The mixture was stirred for 60 h at rt. Aq. sat. NH₄Cl (1 mL) was added and the reaction mixture was extracted with CH₂Cl₂. The organic phase was concentrated, the residue was dissolved in EtOAc (5 mL) and HCl 0.1 N (5 mL) was added. Layers were separated, the organic phase was washed with brine, dried over magnesium sulfate, filtered and concentrated under vacuum. The crude product was purified by chromatography (silica gel saturated with triethylamine, ethyl acetate-hexane) on a RediSep® Gold column using Teledyne Isco purification system to yield 34 mg (67% yield) of product 20 as a white solid. MS (ESI+): 801.74 (M+Na)⁺.

### Example 17

### Production of 9-oxo-20-acetoxy-salinomycin methyl ester (21)

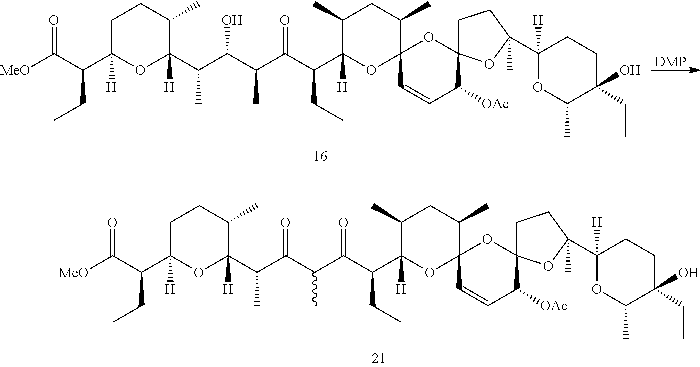

A solution of DMP (64 mg, 2.4 eq.) in CH₂Cl₂ (2 mL) was added to a mixture of (16, 50 mg, 1.0 eq.) (Example 12) and NaHCO₃ (52 mg, 10 eq.) in CH₂Cl₂ (1 mL) at rt. The mixture was stirred for 60 h at rt. Sat. aq. NaHCO₃ (1 mL) and sat. aq. Na₂S₂O₃ (1 mL) was added, the biphasic mixture was stirred for 1 h before the reaction mixture was extracted with CH₂Cl₂. The organic phase was washed with brine, dried over magnesium sulfate, filtered and concentrated under vacuum. The crude product was purified by chromatography (silica gel saturated with triethylamine, ethyl acetate-hexane) on a RediSep® Gold column using Teledyne Isco purification system to yield 40 mg (80% yield) of 21. MS (ESI+): 827.66 (M+Na)⁺.

### Example 18

### Production of 9-11-pyrazole-20-acetoxy-salinomycin methyl ester (22) and 9-11-pyrazole-salinomycin methyl ester (23)

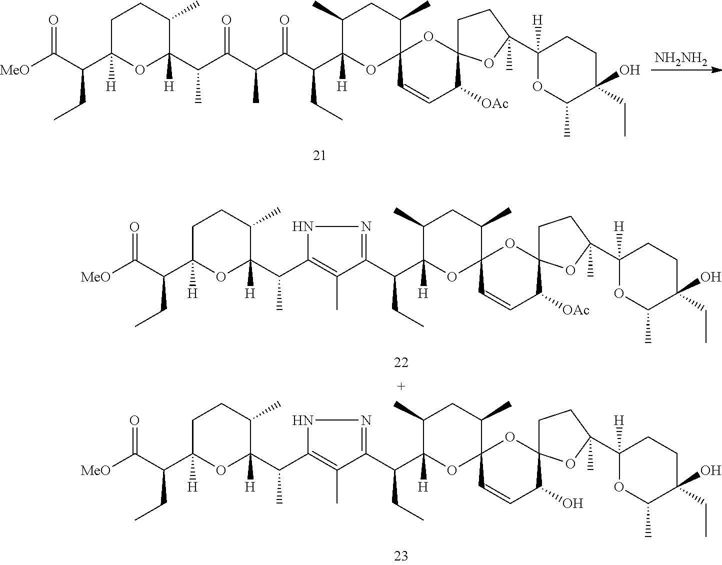

9-Oxo-20-acetoxy-salinomycin methyl ester (21, 216 mg, 1.0 eq.) (Example 17) and anhydrous sodium sulfate were mixed in Et₂O (2.5 mL) at rt and hydrazine hydrate (24 μL, 1.5 eq.) was added. The mixture was stirred for 20 h at rt and was concentrated under reduced pressure. The crude product was purified by chromatography (silica gel saturated with triethylamine, ethyl acetate-hexane) on a RediSep® Gold column using Teledyne Isco purification system to yield 133 mg (66% yield) of product 22, MS (ESI+): 801.67 (M+H)⁺ and 25 mg (12% yield) of product 23, MS (ESI+): 759.58 (M+H)⁺.

### Example 19

### Production of 9-11-pyrazole-20-acetoxy-salinomycin sodium salt(24)

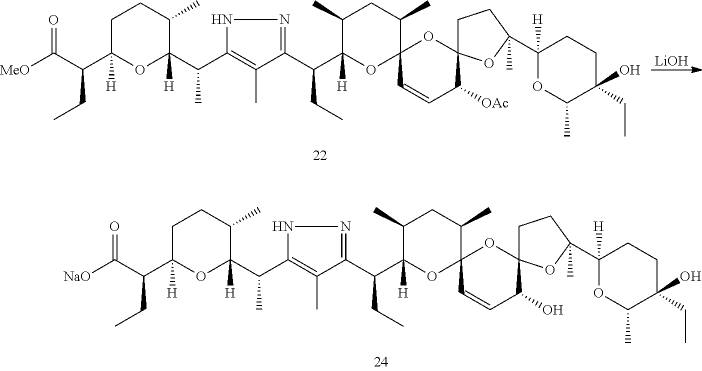

9-11-pyrazole-20-acetoxy-salinomycin methyl ester (22, 43 mg, 1.0 eq.) (Example 18) and lithium hydroxide hydrate (49 mg, 22 eq.) were mixed in THF (1.0 mL), MeOH (0.5 mL) and water (0.5 mL) at rt. The mixture was stirred for 2 h at rt and was heated to 50° C. for 64 h. The mixture was cooled to rt, water (5 mL) and hexane (5 mL) were added and phases were separated. The organic phase was washed with HCl 0.1 N (5 mL), brine (5 mL), aq. sat. NaHCO₃ (2×5 mL) and brine (5 mL). The organic phase was dried over sodium sulfate, filtered and concentrated under vacuum. The crude product was purified by chromatography (silica gel, ethyl acetate-hexane-acetone) on a RediSep® Gold column using Teledyne Isco purification system to yield 15 mg (43% yield) of 24. MS (ESI+): 745.62 (M+H)⁺.

### Example 20

### Production of 20-acetoxy-salinomycin dimethylamide (25)

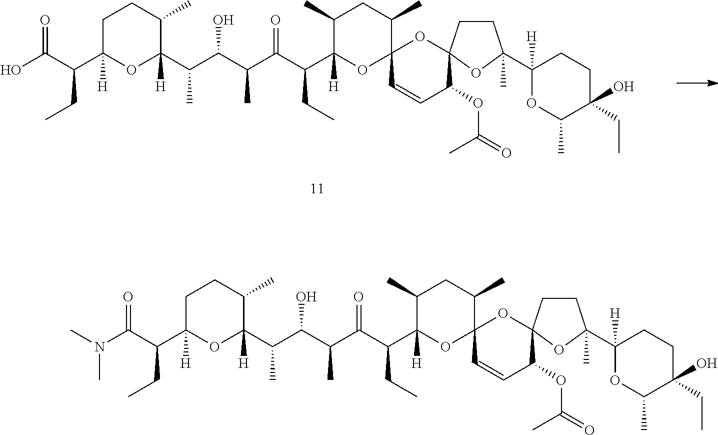 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C ; CC[C@@H](C(=O)[C@@H](C)[C@@H](O)[C@H](C)[C@@H]1O[C@@H]([C@@H](CC)C(=O)N(C)C)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C]

To a solution of salinomycin acetate (11, 0.1 g, 0.13 mmol) (Example 7) in dry DCM (3 ml) were added dimethyl amine (0.3 ml, 2M in THF), followed by addition of PyBrop (0.06 g, 0.13 mmol) and the reaction was stirred at ambient temperature under nitrogen for 18 h. Afterwards, the reaction was diluted with ethyl acetate and washed with saturated sodium bicarbonate. The organic layer was dried over magnesium sulfate and concentrated in vacuo. The crude product was purified by flash chromatography using a 12M Biotage silica column using ethyl acetatehexane gradient. The pure product 25 was isolated in 28% yield. MS 842.6 (M+Na); calc. exact mass 819.56.

### Example 21

### Production of 20-acetoxy-salinomycin methylamide (26)

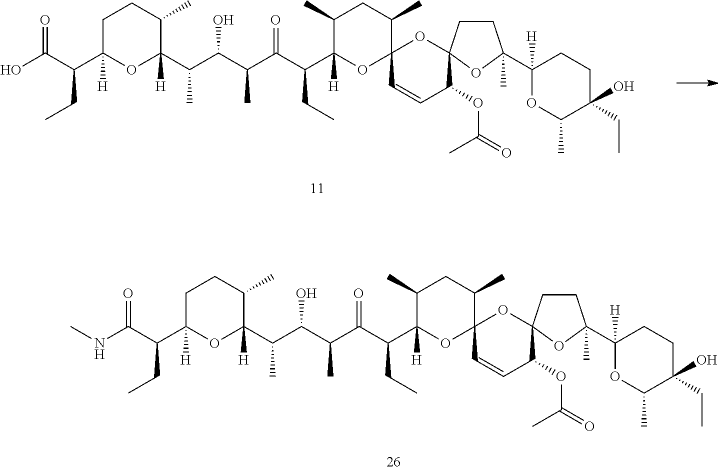 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C ; CC[C@@H](C(=O)[C@@H](C)[C@@H](O)[C@H](C)[C@@H]1O[C@@H]([C@@H](CC)C(=O)NC)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C]

Salinomycin amide 26 was prepared analogously to amide 25 (Example 20). The product 26 was isolated in 46% yield after chromatography. MS 828.5 (M+Na); calc. exact mass 805.5.

### Example 22

### Production of Salinomycin Dimethylamide (27)

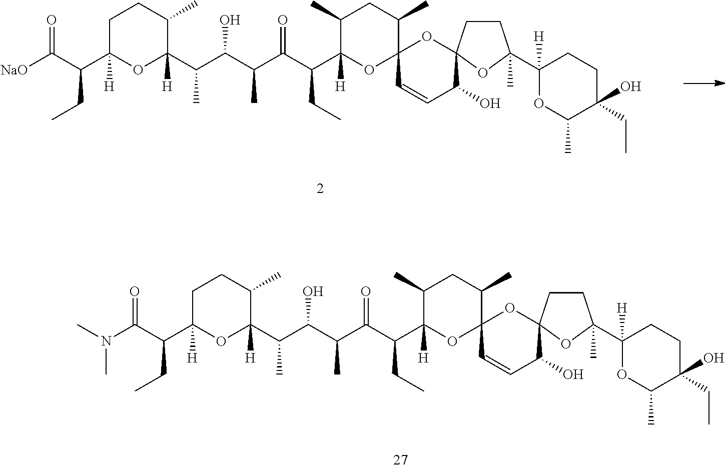

To a solution of 0.2 g (0.27 mmol) salinomycin in dry DCM (5 ml) were added dimethyl amine (0.6 ml, 2M in THF), followed by addition of PyBrop (0.125 g, 0.27 mmol) and the reaction was stirred at ambient temperature under nitrogen for 18 h. Afterwards, the reaction was diluted with ethyl acetate and washed with saturated sodium bicarbonate. The organic layer was dried over magnesium sulfate and concentrated in vacuo. The crude product was purified by flash chromatography using a 25SM Biotage silica column using ethyl acetatehexane gradient. The pure product 27 was isolated in 38% yield. MS 800.5 (M+Na); calc. exact mass 777.5.

### Example 23

### Production of Salinomycin Methylamide (28)

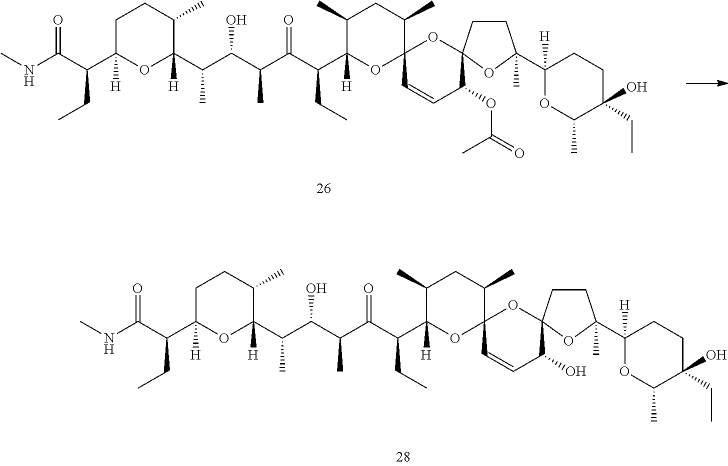 [SMILES: CC[C@@H](C(=O)[C@@H](C)[C@@H](O)[C@H](C)[C@@H]1O[C@@H]([C@@H](CC)C(=O)NC)CC[C@@H]1C)[C@H]1O[C@]2(C=C[C@@H](OC(C)=O)[C@]3(CC[C@@](C)([C@H]4CC[C@](O)(CC)[C@H](C)O4)O3)O2)[C@H](C)C[C@@H]1C ; CC[C@@H](C(=O)[C@@H](C)[C@@H](O)[C@H](C)[C@@H]1O[C@@H]([C@@H](CC)C(=O)NC)CC[C@@H]1C)[C@H]1O[C@]2(C=C[C@@H](O)[C@]3(CC[C@@](C)([C@H]4CC[C@](O)(CC)[C@H](C)O4)O3)O2)[C@H](C)C[C@@H]1C]

To a solution of 0.07 g (0.09 mmol) salinomycin amide 26 (Example 25) in dry methanol (3 ml) was added anhydrous potassium carbonate (0.06 g, 0.43 mmol) and the reaction was stirred at ambient temperature under nitrogen for 4 h. Afterwards, the reaction mixture was concentrated in vacuo and re-diluted with ethyl acetate. It was washed with brine, dried over magnesium sulfate and concentrated in vacuo. The crude product was purified by flash chromatography using a 12M Biotage silica column using ethyl acetatehexane gradient. The pure product 28 was isolated in 17% yield. MS 786.5 (M+Na); calc. exact mass 763.5.

### Example 24

### Proliferation Assay (for HMLE GFP/Ecad, SUM159, Hs578T)

To determine the potency of compounds at inhibiting cellular viability, cells are typically plated in 96-well or 384-well plate formats overnight, treated with compounds for 72 hours in a dose dependent fashion and then assayed with the Promega Cell Titer Glo kit according to manufacturer's instructions; this assay uses a luciferase-based reaction that correlates the amount of ATP present in the cell with the amount of light produced. Luminescent counts read on an EnVision plate reader are then normalized as a % of untreated, DMSO controls to determine the % viability at each dose. These data are plotted with Prism GraphPad software as a function of the log dose and are fitted with a non-linear regression curve to calculate the Effective Concentration at 50% (EC₅₀) and/or Inhibitory Concentration at 50% (IC₅₀).

### Example 25

### ALDEFLUOR Assay

The ALDEFLUOR assay is described in U.S. Pat. Nos. 5,876,956; 6,627,759; 6,537,807; and 6,991,897.

### Summary and Principle

Aldagen's ALDEFLUOR is used to identify, evaluate, and isolate stem and progenitor cells that express high levels of aldehyde dehydrogenase (ALDHbright or ALDH^{br}). The fluorescent ALDEFLOUR Reagent freely diffuses into cells and is a non-toxic substrate for ALDH. The Amount of fluorescent ALDH reaction product that accumulates in cells directly correlates to the ALDH activity in these cells. The negative charge of this reaction product prohibits diffusion from the cells, however it can be actively pumped (effluxed) from cells via the ATP-binding cassettes (ABC) transporter system. This active efflux is inhibited by the special formulation of the ALDEFLUOR Assay Buffer. Therefore, the ALDEFLUOR reaction product will be retained only by cells with intact membranes and fixed, permeabilized or dead cells will appear ALDH negative.

With the ALDEFLUOR assay, viable stem cell and progenitor cells are identified by flow cytometry as cells with higher expression of ALDH. Such cells are recognized by comparing the fluorescence in a test sample to that in a control containing diethylaminobenzaldehyde (DEAB), a specific inhibitor of ALDH. The assay reaction is then measured in the green fluorescence channel of a standard flow cytometer.

The ALDEFLUOR reagent is provided in a stable, inactive form (BODIPY®-aminoacetaldehyde-diethyl acetate, BAAA-DA). For use, dry ALDEFLUOR reagent is dissolved in DMSO, converted to the active substrate (BODIPY-aminoacetaldehyde, BAAA) by treatment with 2N HCl and diluted to the working concentration with ALDEFLUOR Assay Buffer. To perform the assay, an aliquot of the activated substrate is added to the cells suspended in ALDEFLUOR Assay Buffer. An aliquot of this cell mixture is immediately transferred to a tube containing DEAB for the control. These mixtures are incubated to allow conversion of the substrate to the fluorescent product (BODIPY-aminoacetate, BAA). The amount of intracellular fluorescent product is then measured using a flow cytometer.

### Protocol

All necessary supplies (e.g., reagents) were first assembled and allowed to come to room temperature (RT), 18 to 22° C. before use. The ALDEFLUOR reagent was then activated by 1) adding 25 μl of DMSO to the vial of dry ALDEFLUOR reagent. The mixture was then mixed well. 2) The mixture was allowed to stand for 1 min at RT. 3) 25 μl of 2N HCl was then added and mixed well. 4) The mixture was then incubated for 15 min at RT. Do NOT exceed 30 minutes. 360 μl of ALDEFLUOR Assay Buffer was added to the vial and mixed. The activated reagent was kept at 2 to 8° C. during use. The remaining activated ALDEFLUOR substrate was stored at or below −20° C.

Fresh or frozen test samples were prepared according to standard procedures. Cells were lysed, then centrifuged for 5 min at 250×g. The supernatant was then removed and the cells suspended in 1 mL of ALDEFLUOR Assay Buffer. A cell count was then performed, and the cell concentration was adjusted to 1×10⁶ cells/mL with ALDEFLUOR Assay Buffer.

One “test” and one “control” 12×75 mm tube was labelled for each sample to be tested. To a sample, 5 uL of DEAB solution was added. The control tube and DEAB vial were recapped immediately. 5 uL activated ALDEFLUOR substrate was then added per milliliter of sample to a “test” tube. 0.5 mL of the sample was then mixed and immediately transferred to the DEAB “control” tube. The control and substrate solution described were then added for each sample to be tested. The “control” and “test” samples were incubated for 30 to 60 minutes at 37° C. (not to exceed 60 minutes). Following incubation, the tubes were centrifuged for 5 minutes at 250×g. The supernatant was then removed and the cell pellets resuspended in 0.5 mL of ALDEFLUOR Assay Buffer. Samples were capped and placed on ice or in the refrigerator immediately. Samples were stable for 24 hours at 2 to 8° C. A flow cytometer instrument was then set-up according to the manufacturer's instructions. At least 100,000 events were acquired per sample.

To obtain read-out, the DEAB control sample was placed on the cytometer in set-up mode. FSC and SSC voltages were adjusted and gains set to center the nucleated cell population within the FSC vs. SSC plot. R2 region was adjusted to encompass the nucleated cell population based on scatter. On the FL1 vs. SSC plot, the FL1 photo-multiplier tube voltage was adjust so that the right edge of the stained population is placed at the second log decade on the dot plot. The tube was then removed. The corresponding ALDH test sample was then placed on the cytometer. A region R2 was then created to encompass the cell population that is ALDH^{br}, and the tube was removed. For data acquisition of test samples: the analyzer was removed from set-up mode, and 100,000 events were collected in R1 for each ALDH and DEAB sample using the same instrument settings.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A compound of Formula I: 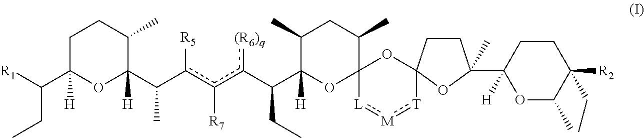
wherein,
R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano;
R₁, together with R₃, R₅, or R₆, and the atoms to which they are attached, may optionally form a heterocyclyl ring;
R₂ is —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀;
L-M-T together form a structure selected from —C(R₃)ₙ—C(R₃)ₙ—C(R₃)ₙ—, —CR₃═CR₃—C(R₃)ₙ—, and —C(R₃)ₙ—CR₃═CR₃—; or L-M, M-T, or L-M-T, and one to three additional —C(R₃)ₙ—, —O—, —NR₁₁—, or —S— to which they are bound, together form a 3-6 membered cyclyl, heterocyclyl, aryl, or heteroaryl ring, wherein n is independently 1 or 2;
each R₃ is independently H, halo, oxo, —OR₁₀, —SR₁₀, —OC(O)R₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl;
R₅ is H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀;
R₆ is independently H, oxo, —OR₁₀, —SR₁₀, —COR₁₀, —CNR₁₁R₁₂, —C(O)R₁₀, —C(O)OR₁₀, —C(O)NR₁₁R₁₂, —OC(O)R₁₀, —OS(O)R₁₀, —OC(O)OR₁₀, —OS(O)OR₁₀, —NR₁₁R₁₂, ═NR₁₁, —OC(O)NR₁₁R₁₂, —SC(O)NR₁₁R₁₂, halo (e.g., F, Cl, Br, I), —NH₂, cyano, C₁-C₈ alkyl, cyclylalkyl, or aryl;
R₅ and R₆ together may optionally form a substituted or unsubstituted 5-8 membered cyclyl, heterocyclyl, aryl, or heteroaryl ring;
R₇ is H, halo, C₁-C₈ alkyl, or C₁-C₈ heteroalkyl;
R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain;
R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR₁₃, —C(O)OR₁₃, —OC(O)R₁₃, —C(O)R₁₃, —S(O)R₁₃, —S(O₂)R₁₃, —NR₁₃1R₁₄, cyano, or an amino acid side chain;
R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano;
R₁₅ and R₁₆ are each independently ═O, —S, —OR₁₇, —SR_{N}, or —NR₁₇R₁₈, provided R₁₅ and R₁₆ are not both double-bonded moieties;
R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl;
R₁₉ is —O—, —S—, —NR₁₇—, —N(OH)—, or —N(OR₁₀)—; and
q is 1 or 2;
provided that when R₁ is —C(O)OH, n is 2, q is 1, R₆ is oxo, and R₇ is methyl, R₂, R₃, and R₅ are not all hydroxy; provided that when R₁ is —C(O)OH, R₆ is oxo, R₇ is methyl, and R₃ and R₅ are hydroxy, R₂ is not benzoxy or benzyloxy; and provided that when R₁ is —C(O)OH, R₆ is oxo, R₇ is methyl, and R₂ is hydroxyl, R₃ or R₅ are not —OCH₂Cl, —OCH₂Br, or —OC(O)CH₂Cl.

2. The compound of claim 1, wherein the compound is a compound of Formula II: 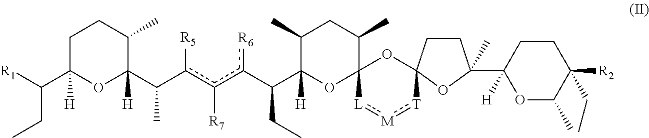
wherein,
R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano;
R₂ is —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀;
L-M-T together form a structure selected from —C(R₃)₂—C(R₃)₂—C(R₃)₂—, —CR₃═CR₃—C(R₃)₂—, and —C(R₃)₂—CR₃═CR₃—; or L-M, M-T, or L-M-T, and one to three additional —C(R₃)₂—, —O—, —NR₁₁—, or —S— to which they are bound, together form a 3-6 membered cyclyl, heterocyclyl, aryl, or heteroaryl ring;
each R₃ is independently H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, cyclyl, heterocyclyl, aryl, heteroaryl, arylalkyl, or heteroarylalkyl;
R₅ is H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀;
R₆ is H, oxo, —OR₁₀, —SR₁₀, —OC(O)R₁₀, —OS(O)R₁₀, —OC(O)OR₁₀, —OS(O)OR₁₀, —NR₁₁R₁₂, ═NR₁₁, —OC(O)NR₁₁R₁₂, —SC(O)NR₁₁R₁₂, halo (e.g., F, Cl, Br, I), —NH₂, cyano, cyclylalkyl, or aryl;
R₅ and R₆ together may optionally form a substituted or unsubstituted 5-8 membered cyclyl, heterocyclyl, aryl, or heteroaryl ring;
R₇ is H, halo, C₁-C₈ alkyl, or C₁-C₈ heteroalkyl;
R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain;
R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR₁₃, —C(O)OR₁₃, —OC(O)R₁₃, —C(O)R₁₃, —S(O)R₁₃, —S(O₂)R₁₃, —NR₁₃R₁₄, cyano, or an amino acid side chain;
R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano;
R₁₅ and R₁₆ are each independently ═O, ═S, —OR₁₇, —SR₁₇, or —NR₁₇R₁₈, provided R₁₅ and R₁₆ are not both double-bonded moieties;
R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl; and
R₁₉ is —O—, —S—, —NR₁₇—, —N(OH)—, or —N(OR₁₀)—,
provided that when R₁ is —C(O)OH, R₆ is oxo, and R₇ is methyl, R₂, R₃, and R₅ are not all hydroxy; provided that when R₁ is —C(O)OH, R₆ is oxo, R₇ is methyl, and R₃ and R₅ are hydroxy, R₂ is not benzoxy or benzyloxy; and provided that when R₁ is —C(O)OH, R₆ is oxo, R₇ is methyl, and R₂ is hydroxyl, R₃ or R₅ are not —OCH₂Cl, —OCH₂Br, or —OC(O)CH₂Cl.

3. The compound of claim 2, wherein R₁ is —CH₂OR₁₀, —C(O)R₁₀, —C(O)OR₁₀, or —C(O)NR₁₁R₁₂.

4. The compound of claim 2, wherein R₁ is heteroaryl.

5. The compound of claim 2, wherein R₂ is —OR₁₀.

6. The compound of claim 2, wherein R₂ is hydroxy.

7. The compound of claim 2, wherein L-M-T is —CR₃═CR₃—C(R₃)₂—.

8. The compound of claim 2, wherein R₃ is —OR₁₀, —OC(O)R₁₀, —OS(O)R₁₀, —OC(O)OR₁₀, —OS(O)OR₁₀, —OC(O)NR₁₁R₁₂, or —OP(R₁₅R₁₆)OR₁₀.

9. The compound of claim 8, wherein R₃ is —OC(O)R₁₀.

10. The compound of claim 9, wherein R₁₀ is methyl.

11. The compound of claim 2, wherein R₃ is —N₃, —NR₁₁R₁₂, —NNR₁₁, —NR₁₁C(O)OR₁₂, —NR₁₁′C(O)NR₁₁R₁₂, —NR₁₁′S(O₂)NR₁₁R₁₂, or —NHP(R₁₅R₁₆)OR₁₀.

12. The compound of claim 2, wherein R₃ is halo.

13. The compound of claim 2, wherein R₅ is oxo, —OR_{M}, —OC(O)R₁₀, —OC(O)OR₁₀, or —OC(O)NR₁₁R₁₂.

14. The compound of claim 2, wherein R₆ is oxo, —OR_{M}, —OC(O)R₁₀, —OC(O)OR₁₀, or —OC(O)NR₁₁R₁₂.

15. The compound of claim 2, wherein R₆ is —NR₁₁R₁₂ or ═NR₁₁.

16. The compound of claim 2, wherein R₅ and R₆ together form a substituted or unsubstituted 5-membered heteroaryl ring.

17. The compound of claim 2, wherein R₇ is methyl.

18. The compound of claim 1, wherein the compound is 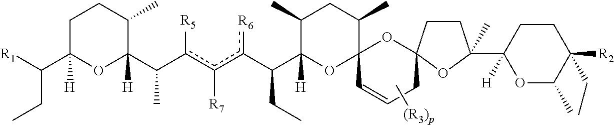
wherein R₁, R₂, R₅, R₆, R₇, R₈, R₉, R₁₀, R₁₁, R₁₂, R₁₃, R₁₄, R₁₅, R₁₆, R₁₇, R₁₈ and R₁₉ are as defined in claim 1;
each R₃ is independently halo, oxo, —OR₁₀, —SR₁₀, —OC(O)R₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl; and
p is 0-4.

19. The compound of claim 18, wherein the compound is 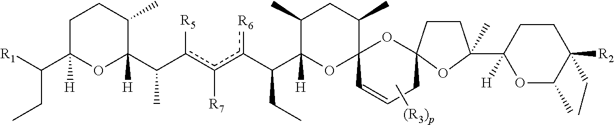
wherein R₁, R₂, R₅, R₆, R₇, R₈, R₉, R₁₀, R₁₁, R₁₂, R₁₃, R₁₄, R₁₅, R₁₆, R₁₇, R₁₈ and R₁₉ are as defined in claim 2;
each R₃ is independently halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, cyclyl, heterocyclyl, aryl, heteroaryl, arylalkyl, or heteroarylalkyl; and
p is 0-4.

20. The compound of claim 19, wherein R₇ is methyl.

21. The compound of claim 19, wherein R₂ is —OH.

22. The compound of claim 19, wherein R₁ is —CH₂OR₁₀, —CH₂NR₁₁R₁₂, —C(O)R₁₀, —C(O)OR₁₀, or —C(O)NR₁₁R₁₂.

23. The compound of claim 22, wherein R₁ is —COOH.

24. The compound of claim 1, wherein the compound is 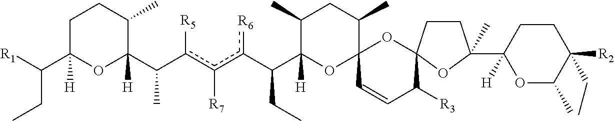
wherein R₁, R₂, R₅, R₆, R₇, R₈, R₉, R₁₀, R₁₁, R₁₂, R₁₃, R₁₄, R₁₅, R₁₆, R₁₇, R₁₈ and R₁₉ are as defined in claim 1; and
each R₃ is independently halo, oxo, —OR₁₀, —SR₁₀, —OC(O)R₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl.

25. The compound of claim 24, wherein the compound is 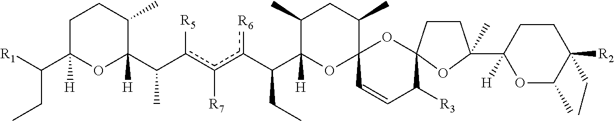
wherein R₁, R₂, R₅, R₆, R₇, R₈, R₉, R₁₀, R₁₁, R₁₂, R₁₃, R₁₄, R₁₅, R₁₆, R₁₇, R₁₈ and R₁₉ are as defined in claim 2; and
each R₃ is independently halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, cyclyl, heterocyclyl, aryl, heteroaryl, arylalkyl, or heteroarylalkyl.

26. The compound of claim 25, wherein R₇ is methyl.

27. The compound of claim 25, wherein R₂ is —OH.

28. The compound of claim 25, wherein R₁ is —CH₂OR₁₀, —CH₂NR₁₁R₁₂, —C(O)R₁₀, —C(O)OR₁₀, or —C(O)NR₁₁R₁₂.

29. The compound of claim 28, wherein R₁ is —COOH.

30. The compound of claim 1, wherein the compound is a compound of Formula II: 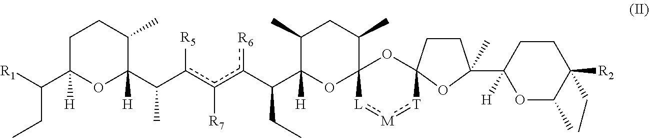
wherein,
R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano;
R₂ is —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀;
L-M-T together form a structure selected from —C(R₃)₂—C(R₃)₂—C(R₃)₂— and —C(R₃)₂—CR₃═CR₃—; or L-M, M-T, or L-M-T, and one to three additional —C(R₃)₂—, —O—, —NR₁₁—, or —S— to which they are bound, together form a 3-6 membered cyclyl, heterocyclyl, aryl, or heteroaryl ring;
each R₃ is independently H, halo, oxo, —OR₁₀, —SR₁₀, —OC(O)R₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁'S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl;
R₅ is H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁'S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀;
R₆ is H, oxo, —OR₁₀, —SR₁₀, —COR₁₀, —CNR₁₁R₁₂, —C(O)R₁₀, —C(O)OR₁₀, —C(O)NR₁₁R₁₂, —OC(O)R₁₀, —OS(O)R₁₀, —OC(O)OR₁₀, —OS(O)OR₁₀, —OC(O)NR₁₁R₁₂, —SC(O)NR₁₁R₁₂, halo (e.g., F, Cl, Br, I), —NH₂, cyano, C₁-C₈ alkyl, cyclylalkyl, or aryl;
R₅ and R₆ together may optionally form a substituted or unsubstituted 5-8 membered cyclyl, heterocyclyl, aryl, or heteroaryl ring;
R₇ is H, halo, C₁-C₈ alkyl, or C₁-C₈ heteroalkyl;
R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain;
R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR₁₃, —C(O)OR₁₃, —OC(O)R₁₃, —C(O)R₁₃, —S(O)R₁₃, —S(O₂)R₁₃, —NR₁₃R₁₄, cyano, or an amino acid side chain;
R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano;
R₁₅ and R₁₆ are each independently ═O, ═S, —OR₁₇, —SR₁₇, or —NR₁₇R₁₈ provided R₁₅ and R₁₆ are not both double-bonded moieties;
R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl; and
R₁₉ is —O—, —S—, —NR₁₇—, —N(OH)—, or —N(OR₁₀).

31. The compound of claim 30, wherein the compound is a compound of Formula II: 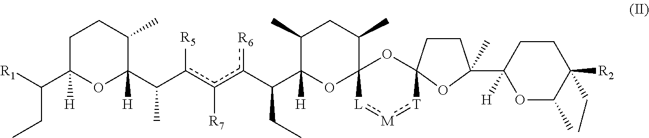
wherein L, M, T, R₁, R₂, R₅, R₆, R₇, R₈, R₉, R₁₀, R₁₁, R₁₂, R₁₃, R₁₄, R₁₅, R₁₆, R₁₇, R₁₈ and R₁₉ are as defined in claim 30; and
each R₃ is independently H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, cyclyl, heterocyclyl, aryl, heteroaryl, arylalkyl, or heteroarylalkyl.

32. The compound of claim 1, wherein the compound is a compound of Formula II: 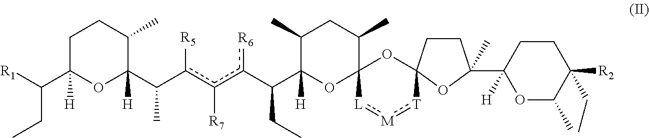
wherein,
R₁ is —OR₁₀, —CH₂OR₁₀, —CH₂NR₁₁R₁₂, —C(O)R₁₀, —C(O)OR₁₀, —C(O)NR₁₁R₁₂, —OC(O)R₁₀, —OC(O)OR₁₀, —NR₁₁R₁₂, —OC(O)NR₁₁R₁₂, oxo, C₁-C₈ alkyl, C₁-C₈ heteroalkyl, halo, haloalkyl, cyclyl, heterocyclyl, aryl, heteroaryl, arylalkyl, heteroarylalkyl, nitro, or cyano;
R₂ is —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —SC(O)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀;
L-M-T together form a structure selected from —C(R₃)₂—C(R₃)₂—C(R₃)₂—, —CR₃═CR₃—C(R₃)₂—, and —C(R₃)₂—CR₃═CR₃—; or L-M, M-T, or L-M-T, and one to three additional —C(R₃)₂—, —O—, —NR₁₁—, or —S— to which they are bound, together form a 3-6 membered cyclyl, heterocyclyl, aryl, or heteroaryl ring;
each R₃ is independently H, halo, oxo, —SR₁₀, —OS(O)R₁₀, —OC(O)OR₁₀, —OS(O)OR₁₀, —OS(O)₂R₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, C₁-C₈ alkyl, C₁-C₈ heteroalkyl, cyclyl, heterocyclyl, aryl, heteroaryl, arylalkyl, or heteroarylalkyl.
R₅ is H, halo, oxo, —OR₁₀, —SR₁₀, —OC(O)R₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁'S(O₂)NR₁₁R₁₂, —P(R₁₅R₁₆)OR₁₀, —OP(R₁₅R₁₆)OR₁₀, —SP(R₁₅R₁₆)OR₁₀, or —NHP(R₁₅R₁₆)OR₁₀;
R₆ is H, oxo, —OR₁₀, —SR₁₀, —COR₁₀, —CNR₁₁R₁₂, —C(O)R₁₀, —C(O)OR₁₀, —C(O)NR₁₁R₁₂, —OC(O)R₁₀, —OS(O)R₁₀, —OC(O)OR₁₀, —OS(O)OR₁₀, —NR₁₁R₁₂, ═NR₁₁, —OC(O)NR₁₁R₁₂, —SC(O)NR₁₁R₁₂, halo (e.g., F, Cl, Br, I), —NH₂, cyano, C₁-C₈ alkyl, cyclylalkyl, or aryl;
R₅ and R₆ together may optionally form a substituted or unsubstituted 5-8 membered cyclyl, heterocyclyl, aryl, or heteroaryl ring;
R₇ is H, halo, C₁-C₈ alkyl, or C₁-C₈ heteroalkyl;
R₁₀ is H, substituted or unsubstituted C₁-C₈ alkyl, C₂-C₈ alkenyl, C₁-C₈ heteroalkyl, substituted or unsubstituted C₃-C₈ cyclyl, C₃-C₈ heterocyclyl, aryl, heteroaryl, arylalkyl, or heteroarylalkyl, or an amino acid side chain;
R₁₁, R₁₁′, and R₁₂ are each independently H, substituted or unsubstituted C₁-C₈ alkyl, C₁-C₈ heteroalkyl, C₃-C₈ cyclyl, C₃-C₈ heterocyclyl, aryl, heteroaryl, arylalkyl, heteroarylalkyl, —OR₁₃, —C(O)OR₁₃, —OC(O)R₁₃, —C(O)R₁₃, —S(O)R₁₃, —S(O₂)R₁₃, —NR₁₃R₁₄, cyano, or an amino acid side chain;
R₁₃ and R₁₄ are each independently H, C₁-C₈ alkyl, C₁-C₈ heteroalkyl, —C(O)R₁₀, C₃-C₈ cyclyl, C₃-C₈ heterocyclyl, aryl, heteroaryl, arylalkyl, heteroarylalkyl, or cyano;
R₁₅ and R₁₆ are each independently ═O, ═S, —OR₁₇, —SR₁₇, or —NR₁₇R₁₈ provided R₁₅ and R₁₆ are not both double-bonded moieties;
R₁₇ and R₁₈ are each independently H, C₁-C₈ alkyl, C₁-C₈ heteroalkyl, C₃-C₈ cyclyl, C₃-C₈ heterocyclyl, aryl, heteroaryl, arylalkyl, or heteroarylalkyl; and
R₁₉ is —O—, —S—, —NR₁₇—, —N(OH)—, or —N(OR₁₀)—.

33. The compound of claim 32, wherein the compound is a compound of Formula II: 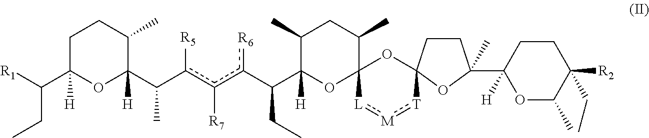
wherein L, M, T, R₁, R₂, R₅, R₆, R₇, R₈, R₉, R₁₀, R₁₁, R₁₂, R₁₃, R₁₄, R₁₅, R₁₆, R₁₇, R₁₈ and R₁₉ are as defined in claim 32; and
each R₃ is H, halo, oxo, —SR₁₀, —OS(O)R₁₀, —OC(O)OR₁₀, —OS(O)OR₁₀, cyano, —N₃, —NR₁₁R₁₂, —NNR₁₁, —OC(O)NR₁₁R₁₂, —NR₁₁C(O)OR₁₂, —NR₁₁′C(O)NR₁₁R₁₂, —SC(O)NR₁₁R₁₂, —NR₁₁′S(O₂)NR₁₁R₁₂, —P(R₁₅R₁₆)OR₁₀, —OP(R₁₅R₁₆)OR₁₀, —SP(R₁₅R₁₆)OR₁₀, —NHP(R₁₅R₁₆)OR₁₀, cyclyl, heterocyclyl, aryl, heteroaryl, arylalkyl, or heteroarylalkyl.

34. The compound of claim 1, wherein the compound is 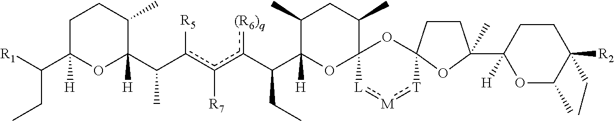
wherein q, n, L, M, T, R₁, R₂, R₃, R₅, R₆, R₇, R₈, R₉, R₁₀, R₁₁, R₁₂, R₁₃, R₁₄, R₁₅, R₁₆, R₁₇, R₁₈ and R₁₉ are as defined in claim 1.

35. A pharmaceutical composition comprising a compound of claim 2.

36. A dosage form comprising a compound or pharmaceutical composition of claim 2.

37. A kit comprising a compound, pharmaceutical composition, or dosage form of claim 2.

38. A method of regulating cell proliferation in a subject in need thereof, comprising administering an effective amount of a compound of claim 2 to the subject, thereby regulating cell proliferation in the subject.

39. A method of treating cancer in a subject comprising administering an effective amount of a compound of claim 2 to the subject.

40. A method of inhibiting proliferation of cancer stem cells, comprising contacting the cancer stem cells with a compound of claim 2, thereby inhibiting the proliferation of cancer stem cells.

41. A method of regulating or reducing the growth of microorganisms in a subject, comprising administering an effective amount of a compound of claim 2 to the subject, thereby regulating or reducing the growth of microorganisms in a subject.
